(19) United States
(12) Patent Application Publication
(10) Pub. No.: US 2008/0312223 A1
(43) Pub. Date: Dec. 18, 2008

(54) Thiazole And Isothiazole Derivatives That Modulate The Activity Of CDK, GSK And Aurora Kinases

(75) Inventors: Valerio Berdini, Cambridge (GB); Michael Alistair O'Brien, Hitchin (GB); Theresa Rachel Phillips, Macclesfield (GB); Paul Graham Wyatt, Perth (GB)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS LIMITED, Cambridge (GB)

(21) Appl. No.: 11/813,173

(22) PCT Filed: Dec. 30, 2005
(86) PCT No.: PCT/GB05/05089

§ 371 Date: Dec. 4, 2007

Related U.S. Application Data
(60) Provisional application No. 60/640,509, filed on Dec. 30, 2004.

(30) Foreign Application Priority Data
Dec. 30, 2004 (GB) 0428549.0

(51) Int. Cl. A61K 31/5377; C07D 277/38; A61K 31/426; A61P 35/02; A61P 35/00; C07D 417/14

(52) U.S. Cl. 514/232.5; 548/194; 514/370; 514/234.5; 544/133; 544/79

(57) ABSTRACT

The invention provides a compound of the formula (I): or a salt, N-oxide, tautomer or solvate thereof, wherein X is CR⁵ or N; each of Q¹ and Q² is a carbon atom; Q³ is selected from S and CH; Q⁴ is selected from CR² and S; provided that one of Q³ and Q⁴ is S and the other of Q³ and Q⁴ is not S; wherein when Q³ is S, there is a double bond between Q¹ and Q⁴ and a double bond between Q² and the adjacent ring nitrogen atom N; and when Q⁴ is S, there is a double bond between Q¹ and Q², and a double bond between Q³ and the adjacent ring nitrogen atom N; A is a bond or —(CH₂)ₘ—(B)ₙ—; B is C═O, NR⁸(C═O) or O(C═O) wherein R¹ is hydrogen or C1_4 hydrocarbyl optionally substituted by hydroxy or C₁₋₄ alkoxy; m is 0, 1 or 2; n is 0 or 1; R^{o} is hydrogen or, together with NR^{g} when present, forms a group —(CH₂)ₚ— wherein p is 2 to 4; R¹ is hydrogen, a carbocyclic or heterocyclic group having from 3 to 12 ring members, or an optionally substituted C₁₋₈ hydrocarbyl group; R² is hydrogen, halogen, methoxy, or a C₁₋₄ hydrocarbyl group optionally substituted by halogen, hydroxyl or methoxy; R³ and R⁴ together with the carbon atoms to which they are attached form an optionally substituted fused carbocyclic or heterocyclic ring having from 5 to 7 ring members of which up to 3 can be heteroatoms selected from N, O and S; and R⁵ is hydrogen, a group R² or a group R¹⁰ wherein R¹⁰ is as defined in the claims. The compounds have activity as inhibitors of cyclin dependent kinases, glycogen synthase kinases and Aurora kinases.

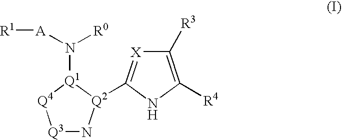

This invention relates to thiazole and isothiazole compounds that inhibit or modulate the activity of Cyclin Dependent Kinases (CDK), Glycogen Synthase Kinases (GSK) and Aurora kinases to the use of the compounds in the treatment or prophylaxis of disease states or conditions mediated by the kinases, and to novel compounds having kinase inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, disease and conditions of the immune system, disease and conditions of the central nervous system, and angiogenesis.

### Cyclin Dependent Kinases

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (cdks) and a diverse set of their cognate protein partners termed cyclins. Cdks are cdc2 (also known as cdk1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific cdk partner proteins.

Modulation of the expression levels, degradation rates, and activation levels of various cdks and cyclins throughout the cell cycle leads to the cyclical formation of a series of cdk/cyclin complexes, in which the cdks are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required cdk/cyclin complex, can lead to cell cycle arrest and/or cellular apoptosis. Aberrant cellular proliferation, as manifested in cancer, can often be attributed to loss of correct cell cycle control. Inhibition of cdk enzymatic activity therefore provides a means by which abnormally dividing cells can have their division arrested and/or be killed. The diversity of cdks, and cdk complexes, and their critical roles in mediating the cell cycle, provides a broad spectrum of potential therapeutic targets selected on the basis of a defined biochemical rationale.

Progression from the G1 phase to the S phase of the cell cycle is primarily regulated by cdk2, cdk3, cdk4 and cdk6 via association with members of the D and E type cyclins. The D-type cyclins appear instrumental in enabling passage beyond the G1 restriction point, where as the cdk2/cyclin E complex is key to the transition from the G1 to S phase. Subsequent progression through S phase and entry into G2 is thought to require the cdk2/cyclin A complex. Both mitosis, and the G2 to M phase transition which triggers it, are regulated by complexes of cdk1 and the A and B type cyclins.

During G1 phase Retinoblastoma protein (Rb), and related pocket proteins such as p130, are substrates for cdk(2, 4, & 6)/cyclin complexes. Progression through G1 is in part facilitated by hyperphosphorylation, and thus inactivation, of Rb and p130 by the cdk(4/6)/cyclin-D complexes. Hyperphosphorylation of Rb and p130 causes the release of transcription factors, such as E2F, and thus the expression of genes necessary for progression through G1 and for entry into S-phase, such as the gene for cyclin E. Expression of cyclin E facilitates formation of the cdk2/cyclin E complex which amplifies, or maintains, E2F levels via further phosphorylation of Rb. The cdk2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the cdk2/cyclin E pathway. Cdk2 is also connected to the p53 mediated DNA damage response pathway via p53 regulation of p21 levels. p21 is a protein inhibitor of cdk2/cyclin E and is thus capable of blocking, or delaying, the G1/S transition. The cdk2/cyclin E complex may thus represent a point at which biochemical stimuli from the Rb, Myc and p53 pathways are to some degree integrated. Cdk2 and/or the cdk2/cyclin E complex therefore represent good targets for therapeutics designed at arresting, or recovering control of, the cell cycle in aberrantly dividing cells.

The exact role of cdk3 in the cell cycle is not clear. As yet no cognate cyclin partner has been identified, but a dominant negative form of cdk3 delayed cells in G1, thereby suggesting that cdk3 has a role in regulating the G1/S transition.

Although most cdks have been implicated in regulation of the cell cycle there is evidence that certain members of the cdk family are involved in other biochemical processes. This is exemplified by cdk5 which is necessary for correct neuronal development and which has also been implicated in the phosphorylation of several neuronal proteins such as Tau, NUDE-1, synapsin1, DARPP32 and the Munc18/Syntaxin1A complex. Neuronal cdk5 is conventionally activated by binding to the p35/p39 proteins. Cdk5 activity can, however, be deregulated by the binding of p25, a truncated version of p35. Conversion of p35 to p25, and subsequent deregulation of cdk5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

Cdk7 is a nuclear protein that has cdc2 CAK activity and binds to cyclin H. Cdk7 has been identified as component of the TFIIH transcriptional complex which has RNA polymerase II C-terminal domain (CTD) activity. This has been associated with the regulation of HIV-1 transcription via a Tat-mediated biochemical pathway. Cdk8 binds cyclin C and has been implicated in the phosphorylation of the CTD of RNA polymerase II. Similarly the cdk9/cyclin-T1 complex (P-TEFb complex) has been implicated in elongation control of RNA polymerase II. PTEF-b is also required for activation of transcription of the HIV-1 genome by the viral transactivator Tat through its interaction with cyclin T1. Cdk7, cdk8, cdk9 and the P-TEFb complex are therefore potential targets for anti-viral therapeutics.

At a molecular level mediation of cdk/cyclin complex activity requires a series of stimulatory and inhibitory phosphorylation, or dephosphorylation, events. Cdk phosphorylation is performed by a group of cdk activating kinases (CAKs) and/or kinases such as wee1, Myt1 and Mik1. Dephosphorylation is performed by phosphatases such as cdc25(a & c), pp2a, or KAP.

Cdk/cyclin complex activity may be further regulated by two families of endogenous cellular proteinaceous inhibitors: the Kip/Cip family, or the INK family. The INK proteins specifically bind cdk4 and cdk6. p16^{ink4} (also known as MTS1) is a potential tumour suppressor gene that is mutated, or deleted, in a large number of primary cancers. The Kip/Cip family contains proteins such as p21^{Cip1,Waf1}, p27^{Kip1} and p57^{kip2}. As discussed previously p21 is induced by p53 and is able to inactivate the cdk2/cyclin(E/A) and cdk4/cyclin(D1/D2/D3) complexes. A typically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of cdks, and their associated proteins, in co-ordinating and driving the cell cycle in proliferating cells have been outlined above. Some of the biochemical pathways in which cdks play a key role have also been described. The development of monotherapies for the treatment of proliferative disorders, such as cancers, using therapeutics targeted generically at cdks, or at specific cdks, is therefore potentially highly desirable. Cdk inhibitors could conceivably also be used to treat other conditions such as viral infections, autoimmune diseases and neuro-degenerative diseases, amongst others. Cdk targeted therapeutics may also provide clinical benefits in the treatment of the previously described diseases when used in combination therapy with either existing, or new, therapeutic agents. Cdk targeted anticancer therapies could potentially have advantages over many current antitumour agents as they would not directly interact with DNA and should therefore reduce the risk of secondary tumour development.

### Diffuse Large B-cell Lymphomas (DLBCL)

Cell cycle progression is regulated by the combined action of cyclins, cyclin-dependent kinases (CDKs), and CDK-inhibitors (CDKi), which are negative cell cycle regulators. p27KIP1 is a CDKi key in cell cycle regulation, whose degradation is required for G1/S transition. In spite of the absence of p27KIP1 expression in proliferating lymphocytes, some aggressive B-cell lymphomas have been reported to show an anomalous p27KIP1 staining. An abnormally high expression of p27KIP1 was found in lymphomas of this type. Analysis of the clinical relevance of these findings showed that a high level of p27KIP1 expression in this type of tumour is an adverse prognostic marker, in both univariate and multivariate analysis. These results show that there is abnormal p27KIP1 expression in Diffuse Large B-cell Lymphomas (DLBCL), with adverse clinical significance, suggesting that this anomalous p27KIP1 protein may be rendered non-functional through interaction with other cell cycle regulator proteins. (Br. J. Cancer. July 1999;80(9):1427-34. p27KIP1 is abnormally expressed in Diffuse Large B-cell Lymphomas and is associated with an adverse clinical outcome. Saez A, Sanchez E, Sanchez-Beato M, Cruz M A, Chacon I, Munoz E, Camacho F I, Martinez-Montero J C, Mollejo M, Garcia J F, Piris Mass. Department of Pathology, Virgen de la Salud Hospital, Toledo, Spain.)

### Chronic Lymphocytic Leukemia

B-Cell chronic lymphocytic leukaemia (CLL) is the most common leukaemia in the Western hemisphere, with approximately 10,000 new cases diagnosed each year (Parker S L, Tong T, Bolden S, Wingo P A: Cancer statistics, 1997. Ca. Cancer. J. Clin. 47:5, (1997)). Relative to other forms of leukaemia, the overall prognosis of CLL is good, with even the most advanced stage patients having a median survival of 3 years.

The addition of fludarabine as initial therapy for symptomatic CLL patients has led to a higher rate of complete responses (27% v 3%) and duration of progression-free survival (33 v 17 months) as compared with previously used alkylator-based therapies. Although attaining a complete clinical response after therapy is the initial step toward improving survival in CLL, the majority of patients either do not attain complete remission or fail to respond to fludarabine. Furthermore, all patients with CLL treated with fludarabine eventually relapse, making its role as a single agent purely palliative (Rai K R, Peterson B, Elias L, Shepherd L, Hines J, Nelson D, Cheson B, Kolitz J, Schiffer C A: A randomized comparison of fludarabine and chlorambucil for patients with previously untreated chronic lymphocytic leukemia. A CALGB SWOG, CTG/NCI—C and ECOG Inter-Group Study. Blood 88:141a, 1996 (abstr 552, suppl 1). Therefore, identifying new agents with novel mechanisms of action that complement fludarabine's cytotoxicity and abrogate the resistance induced by intrinsic CLL drug-resistance factors will be necessary if further advances in the therapy ofthis disease are to be realized.

The most extensively studied, uniformly predictive factor for poor response to therapy and inferior survival in CLL patients is aberrant p53 function, as characterized by point mutations or chromosome 17p13 deletions. Indeed, virtually no responses to either alkylator or purine analog therapy have been documented in multiple single institution case series for those CLL patients with abnormal p53 function. Introduction of a therapeutic agent that has the ability to overcome the drug resistance associated with p53 mutation in CLL would potentially be a major advance for the treatment of the disease.

Flavopiridol and CYC 202, inhibitors of cyclin-dependent kinases induce in vitro apoptosis of malignant cells from B-cell chronic lymphocytic leukemia (B-CLL).

Flavopiridol exposure results in the stimulation of caspase 3 activity and in caspase-dependent cleavage of p27(kip1), a negative regulator of the cell cycle, which is overexpressed in B-CLL (Blood. Nov. 15, 1998;92(10):3804-16 Flavopiridol induces apoptosis in chronic lymphocytic leukemia cells via activation of caspase-3 without evidence of bcl-2 modulation or dependence on functional p53. Byrd J C, Shinn C, Waselenko J K, Fuchs E J, Lehman T A, Nguyen P L, Flinn I W, Diehl L F, Sausville E, Grever M R).

### Aurora Kinases

Relatively recently, a new family of serine/threonine kinases known as the Aurora kinases has been discovered that are involved in the G2 and M phases of the cell cycle, and which are important regulators of mitosis.

The precise role of Aurora kinases has yet to be elucidated but that they play a part in mitotic checkpoint control, chromosome dynamics and cytokinesis (Adams et al., Trends Cell Biol., 11: 49-54 (2001). Aurora kinases are located at the centrosomes of interphase cells, at the poles of the bipolar spindle and in the mid-body of the mitotic apparatus.

Three members of the Aurora kinase family have been found in mammals so far (E. A. Nigg, Nat. Rev. Mol. Cell Biol. 2: 21-32, (2001)). These are:

Aurora A (also referred to in the literature as Aurora 2);

Aurora B (also referred to in the literature as Aurora 1); and

Aurora C (also referred to in the literature as Aurora 3).

The Aurora kinases have highly homologous catalytic domains but differ considerably in their N-terminal portions (Katayama H, Brinkley W R, Sen S.; The Aurora kinases: role in cell transformation and tumorigenesis; Cancer Metastasis Rev. December 2003;22(4):451-64).

The substrates of the Aurora kinases A and B have been identified as including a kinesin-like motor protein, spindle apparatus proteins, histone H3 protein, kinetochore protein and the tumour suppressor protein p53.

Aurora A kinases are believed to be involved in spindle formation and become localised on the centrosome during the early G2 phase where they phosphorylate spindle-associated proteins (Prigent et al., Cell, 114: 531-535 (2003). Hirota et al, Cell, 114:585-598, (2003) found that cells depleted of Aurora A protein kinase were unable to enter mitosis. Furthermore, it has been found (Adams, 2001) that mutation or disruption of the Aurora A gene in various species leads to mitotic abnormalities, including centrosome separation and maturation defects, spindle aberrations and chromosome segregation defects.

The Aurora kinases are generally expressed at a low level in the majority of normal tissues, the exceptions being tissues with a high proportion of dividing cells such as the thymus and testis. However, elevated levels of Aurora kinases have been found in many human cancers (Giet et al., J. Cell. Sci. 112: 3591-361, (1999) and Katayama (2003). Furthermore, Aurora A kinase maps to the chromosome 20q13 region that has frequently been found to be amplified in many human cancers.

Thus, for example, significant Aurora A over-expression has been detected in human breast, ovarian and pancreatic cancers (see Zhou et al., Nat. Genet. 20: 189-193, (1998), Tanaka et al., Cancer Res., 59: 2041-2044, (1999) and Han et al., cancer Res., 62: 2890-2896, (2002).

Moreover, Isola, American Journal of Pathology 147,905-911 (1995) has reported that amplification of the Aurora A locus (20q13) correlates with poor prognosis for patients with node-negative breast cancer.

Amplification and/or over-expression of Aurora-A is observed in human bladder cancers and amplification of Aurora-A is associated with aneuploidy and aggressive clinical behaviour, see Sen et al., J. Natl. Cancer Inst, 94: 1320-1329 (2002).

Elevated expression of Aurora-A has been detected in over 50% of colorectal cancers, (see Bischoff et al., EMBO J., 17: 3052-3065, (1998) and Takahashi et al., Jpn. J. Cancer Res., 91: 1007-1014 (2000)) ovarian cancers (see Gritsko et al. Clin. Cancer Res., 9: 1420-1426 (2003), and gastric tumours Sakakura et al., British Journal of Cancer, 84: 824-831 (2001).

Tanaka et al. Cancer Research, 59: 2041-2044 (1999) found evidence of over-expression of Aurora A in 94% of invasive duct adenocarcinomas of the breast.

High levels of Aurora A kinase have also been found in renal, cervical, neuroblastoma, melanoma, lymphoma, pancreatic and prostate tumour cell lines Bischoff et al. (1998), EMBO J., 17: 3052-3065 (1998); Kimura et al. J. Biol. Chem., 274: 7334-7340 (1999); Zhou et al., Nature Genetics, 20: 189-193 (1998); Li et al., Clin Cancer Res. 9 (3): 991-7 (2003)].

Aurora-B is highly expressed in multiple human tumour cell lines, including leukemic cells [Katayama et al., Gene 244: 1-7)]. Levels of this enzyme increase as a function of Duke's stage in primary colorectal cancers [Katayama et al., J. Natl Cancer Inst., 91: 1160-1162 (1999)].

High levels of Aurora-3 (Aurora-C) have been detected in several tumour cell lines, even though this kinase tends to be restricted to germ cells in normal tissues (see Kimura et al. Journal of Biological Chemistry, 274: 7334-7340 (1999)). Over-expression of Aurora-3 in approximately 50% of colorectal cancers has also been reported in the article by Takahashi et al., Jpn J. Cancer Res. 91: 1007-1014 (2001)].

Other reports of the role of Aurora kinases in proliferative disorders may be found in Bischoff et al., Trends in Cell Biology 9: 454-459 (1999); Giet et al. Journal of Cell Science, 112: 3591-3601 (1999) and Dutertre, et al. Oncogene, 21: 6175-6183 (2002).

Royce et al report that the expression of the Aurora 2 gene (known as STK15 or BTAK) has been noted in approximately one-fourth of primary breast tumours. (Royce M E, Xia W, Sahin A A, Katayama H, Johnston D A, Hortobagyi G, Sen S, Hung M C; STK15/Aurora-A expression in primary breast tumours is correlated with nuclear grade but not with prognosis; Cancer. Jan. 1, 2004;100(1): 12-9).

Endometrial carcinoma (EC) comprises at least two types of cancer: endometrioid carcinomas (EECs) are estrogen-related tumours, which are frequently euploid and have a good prognosis. Nonendometrioid carcinomas (NEECs; serous and clear cell forms) are not estrogen related, are frequently aneuploid, and are clinically aggressive. It has also been found that Aurora was amplified in 55.5% of NEECs but not in any EECs (P<or=0.001) (Moreno-Bueno G, Sanchez-Estevez C, Cassia R, Rodriguez-Perales S, Diaz-Uriarte R, Dominguez O, Hardisson D, Andujar M, Prat J, Matias-Guiu X, Cigudosa J C, Palacios J. Cancer Res. Sep. 15, 2003;63(18):5697-702).

Reichardt et al (Oncol Rep. September-October 2003; 10(5):1275-9)have reported that quantitative DNA analysis by PCR to search for Aurora amplification in gliomas revealed that five out of 16 tumours (31%) of different WHO grade (1×grade II, 1×grade III, 3×grade IV) showed DNA amplification of the Aurora 2 gene. It was hypothesized that amplification of the Aurora 2 gene may be a non-random genetic alteration in human gliomas playing a role in the genetic pathways of tumouri genesis.

Results by Hamada et al (Br. J. Haematol. May 2003; 121(3):439-47) also suggest that Aurora 2 is an effective candidate to indicate not only disease activity but also tumourigenesis of non-Hodgkin's lymphoma. Retardation of tumour cell growth resulting from the restriction of this gene's functions could be a therapeutic approach for non-Hodgkin's lymphoma.

In a study by Gritsko et al (Clin Cancer Res. April 2003; 9(4):1420-6)), the kinase activity and protein levels of Aurora A were examined in 92 patients with primary ovarian tumours. In vitro kinase analyses revealed elevated Aurora A kinase activity in 44 cases (48%). Increased Aurora A protein levels were detected in 52 (57%) specimens. High protein levels of Aurora A correlated well with elevated kinase activity.

Results obtained by Li et al (Clin. Cancer Res. March 2003; 9(3):991-7) showed that the Aurora A gene is overexpressed in pancreatic tumours and carcinoma cell lines and suggest that overexpression of Aurora A may play a role in pancreatic carcinogenesis.

Similarly, it has been shown that Aurora A gene amplification and associated increased expression of the mitotic kinase it encodes are associated with aneuploidy and aggressive clinical behaviour in human bladder cancer. (J. Natl. Cancer Inst. Sep. 4, 2002; 94(17):1320-9).

Investigation by several groups (Dutertre S, Prigent C., Aurora-A overexpression leads to override of the microtubule-kinetochore attachment checkpoint; Mol. Interv. May 2003; 3(3):127-30 and Anand S, Penrhyn-Lowe S, Venkitaraman A R.,

Aurora-A amplification overrides the mitotic spindle assembly checkpoint, inducing resistance to Taxol, Cancer Cell. January 2003; 3(1):51-62) suggests that overexpression of Aurora kinase activity is associated with resistance to some current cancer therapies. For example overexpression of Aurora A in mouse embryo fibroblasts can reduce the sensitivity of these cells to the cytotoxic effects of taxane derivatives. Therefore Aurora kinase inhibitors may find particular use in patients who have developed reistance to existing therapies.

On the basis of work carried out to date, it is envisaged that inhibition of Aurora kinases, particularly Aurora kinase A and Aurora kinase B, will prove an effective means of arresting tumour development.

Harrington et al (Nat Med. March 2004; 10(3):262-7) have demonstrated that an inhibitor of the Aurora kinases suppresses tumour growth and induces tumour regression in vivo. In the study, the Aurora kinase inhibitor blocked cancer cell proliferation, and also triggered cell death in a range of cancer cell lines including leukaemic, colorectal and breast cell lines. In addition, it has shown potential for the treatment of leukemia by inducing apoptosis in leukemia cells. VX-680 potently killed treatment-refractory primary Acute Myelogenous Leukemia (AML) cells from patients (Andrews, Oncogene, 2005, 24, 5005-5015).

Cancers which may be particularly amenable to Aurora inhibitors include breast, bladder, colorectal, pancreatic, ovarian, non-Hodgkin's lymphoma, gliomas and nonendometrioid endometrial carcinomas. Leukemias particularly amenable to Aurora inhibitors include Acute Myelogenous Leukemia (AML), chronic myelogenous leukaemia (CML), B-cell lymphoma (Mantle cell), and Acute Lymphoblastic Leukemia (ALL).

### Glycogen Synthase Kinase

Glycogen Synthase Kinase-3 (GSK3) is a serine-threonine kinase that occurs as two ubiquitously expressed isoforms in humans (GSK3α & beta GSK3β). GSK3 has been implicated as having roles in embryonic development, protein synthesis, cell proliferation, cell differentiation, microtubule dynamics, cell motility and cellular apoptosis. As such GSK3 has been implicated in the progression of disease states such as diabetes, cancer, Alzheimer's disease, stroke, epilepsy, motor neuron disease and/or head trauma. Phylogenetically GSK3 is most closely related to the cyclin dependent kinases (CDKs).

The consensus peptide substrate sequence recognised by GSK3 is (Ser/Thr)-X-X-X-(pSer/pThr), where X is any amino acid (at positions (n+1), (n+2), (n+3)) and pSer and pThr are phospho-serine and phospho-threonine respectively (n+4). GSK3 phosphorylates the first serine, or threonine, at position (n). Phospho-serine, or phospho-threonine, at the (n+4) position appear necessary for priming GSK3 to give maximal substrate turnover. Phosphorylation of GSK3α at Ser21, or GSK3β at Ser9, leads to inhibition of GSK3. Mutagenesis and peptide competition studies have led to the model that the phosphorylated N-terminus of GSK3 is able to compete with phospho-peptide substrate (S/TXXXpS/pT) via an autoinhibitory mechanism. There are also data suggesting that GSK3α and GSKβ may be subtly regulated by phosphorylation of tyrosines 279 and 216 respectively. Mutation of these residues to a Phe caused a reduction in in vivo kinase activity. The X-ray crystallographic structure of GSK3β has helped to shed light on all aspects of GSK3 activation and regulation.

GSK3 forms part of the mammalian insulin response pathway and is able to phosphorylate, and thereby inactivate, glycogen synthase. Upregulation of glycogen synthase activity, and thereby glycogen synthesis, through inhibition of GSK3, has thus been considered a potential means of combating type II, or non-insulin-dependent diabetes mellitus (NIDDM): a condition in which body tissues become resistant to insulin stimulation. The cellular insulin response in liver, adipose, or muscle tissues, is triggered by insulin binding to an extracellular insulin receptor. This causes the phosphorylation, and subsequent recruitment to the plasma membrane, of the insulin receptor substrate (IRS) proteins. Further phosphorylation of the IRS proteins initiates recruitment of phosphoinositide-3 kinase (PI3K) to the plasma membrane where it is able to liberate the second messenger phosphatidylinosityl 3,4,5-trisphosphate (PIP3). This facilitates co-localisation of 3-phosphoinositide-dedependent protein kinase 1 (PDK1) and protein kinase B (PKB or Akt) to the membrane, where PDK1 activates PKB. PKB is able to phosphorylate, and thereby inhibit, GSK3α and/or GSKβ through phosphorylation of Ser9, or ser21, respectively. The inhibition of GSK3 then triggers upregulation of glycogen synthase activity. Therapeutic agents able to inhibit GSK3 may thus be able to induce cellular responses akin to those seen on insulin stimulation. A further in vivo substrate of GSK3 is the eukaryotic protein synthesis initiation factor 2B (eIF2B). eIF2B is inactivated via phosphorylation and is thus able to suppress protein biosynthesis. Inhibition of GSK3, e.g. by inactivation of the “mammalian target of rapamycin” protein (mTOR), can thus upregulate protein biosynthesis. Finally there is some evidence for regulation of GSK3 activity via the mitogen activated protein kinase (MAPK) pathway through phosphorylation of GSK3 by kinases such as mitogen activated protein kinase activated protein kinase 1 (MAPKAP-K1 or RSK). These data suggest that GSK3 activity may be modulated by mitogenic, insulin and/or amino acid stimulii.

It has also been shown that GSK3β is a key component in the vertebrate Wnt signalling pathway. This biochemical pathway has been shown to be critical for normal embryonic development and regulates cell proliferation in normal tissues. GSK3 becomes inhibited in response to Wnt stimulii. This can lead to the de-phosphorylation of GSK3 substrates such as Axin, the adenomatous polyposis coli (APC) gene product and β-catenin. Aberrant regulation of the Wnt pathway has been associated with many cancers. Mutations in APC, and/or β-catenin, are common in colorectal cancer and other tumours. β-catenin has also been shown to be of importance in cell adhesion. Thus GSK3 may also modulate cellular adhesion processes to some degree. Apart from the biochemical pathways already described there are also data implicating GSK3 in the regulation of cell division via phosphorylation of cyclin-D1, in the phosphorylation of transcription factors such as c-Jun, CCAAT/enhancer binding protein α (C/EBPα), c-Myc and/or other substrates such as Nuclear Factor of Activated T-cells (NFATc), Heat Shock Factor-1 (HSF-1) and the c-AMP response element binding protein (CREB). GSK3 also appears to play a role, albeit tissue specific, in regulating cellular apoptosis.

The role of GSK3 in modulating cellular apoptosis, via a pro-apoptotic mechanism, may be of particular relevance to medical conditions in which neuronal apoptosis can occur. Examples of these are head trauma, stroke, epilepsy, Alzheimer's and motor neuron diseases, progressive supranuclear palsy, corticobasal degeneration, and Pick's disease. In vitro it has been shown that GSK3 is able to hyper-phosphorylate the microtubule associated protein Tau. Hyperphosphorylation of Tau disrupts its normal binding to microtubules and may also lead to the formation of intra-cellular Tau filaments. It is believed that the progressive accumulation of these filaments leads to eventual neuronal dysfunction and degeneration. Inhibition of Tau phosphorylation, through inhibition of GSK3, may thus provide a means of limiting and/or preventing neurodegenerative effects.

### PRIOR ART

WO 02/34721 from Du Pont discloses a class of indeno [1,2-c]pyrazol-4-ones as inhibitors of cyclin dependent kinases.

WO 01/81348 from Bristol Myers Squibb describes the use of 5-thio-, sulphinyl- and sulphonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (cdks) and hence their use in the treatment of proliferative disorders such as cancer, leukaemia, psoriasis and the like.

WO 99/21845 from Agouron describes 4-aminothiazole derivatives for inhibiting cyclin-dependent kinases (cdks), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group.

WO 01/98290 (Pharmacia & Upjohn) discloses a class of 3-aminocarbonyl-2-carboxamido thiophene derivatives as protein kinase inhibitors. The compounds are stated to have multiple protein kinase activity.

WO 01/53268 and WO 01/02369 from Agouron disclose compounds that mediate or inhibit cell proliferation through the inhibition of protein kinases such as cyclin dependent kinase or tyrosine kinase.

WO 00/39108 and WO 02/00651 (both to Du Pont Pharmaceuticals) describe broad classes of heterocyclic compounds that are inhibitors of trypsin-like serine protease enzymes, especially factor Xa and thrombin. The compounds are stated to be useful as anticoagulants or for the prevention of thromboembolic disorders.

Heterocyclic compounds that have activity against factor Xa are also disclosed in WO 01/1978 Cor Therapeutics) and US 2002/0091116 (Zhu et al.).

WO 03/035065 (Aventis) discloses a broad class of benzimidazole derivatives as protein kinase inhibitors but does not disclose activity against CDK kinases or GSK kinases.

WO 97/36585 and U.S. Pat. No. 5,874,452 (both to Merck) disclose biheteroaryl compounds that are inhibitors of famesyl transferase.

WO 97/12615 (Warner Lambert) discloses benzimidazoles as 15-lipoxygenase inhibitors.

WO 00/02871 (Merck) discloses compounds that have tyrosine kinase inhibiting activity and which are useful as angiogenesis inhibitors useful in treating diseases such as cancer.

EP 0711768 (Mitsui Toatsu) Chemicals discloses benzimidazole-containing compounds that have activity as anti-cancer agents, anti-viral agents or anti-microbial agents.

WO 03/066629 (Vertex Pharmaceuticals) discloses benzimidazole compounds and analogues thereof as inhibitors of GSK-3.

EP 1460 067 (Takeda) discloses compounds having tyrosine-kinase inhibiting activity.

WO 97/12617 (Warner Lambert) discloses compounds that are lipoxygenase inhibitors and which can be used in treating inflammatory disease, atherosclerosis and restenosis.

WO 2004/041277 (Merck) discloses benzimidazole derivatives as androgen receptor modulators.

WO 01/68585 (Fujisawa Pharmaceutical) discloses amide compounds that have 5-HT antagonist activity and are therefore useful in treating various CNS related disorders.

### SUMMARY OF THE INVENTION

The invention provides compounds that have cyclin dependent kinase inhibiting or modulating activity and glycogen synthase kinase-3 (GSK3) inhibiting or modulating activity, and/or Aurora kinase inhibiting or modulating activity, and which it is envisaged will be useful in preventing or treating disease states or conditions mediated by the kinases.

Thus, for example, it is envisaged that the compounds of the invention will be useful in alleviating or reducing the incidence of cancer.

Accordingly, in a first aspect, the invention provides a compound of the general formula (I):

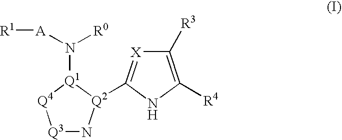

wherein
X is CR⁵ or N;
each of Q¹ and Q² is a carbon atom;
Q³ is selected from S and CH;
Q⁴ is selected from CR² and S; provided that one of Q³ and Q⁴ is S and the other of Q³ and Q⁴ is not S;
wherein when Q³ is S, there is a double bond between Q¹ and Q⁴ and a double bond between Q² and the adjacent ring nitrogen atom N; and when Q⁴ is S, there is a double bond between Q¹ and Q², and a double bond between Q³ and the adjacent ring nitrogen atom N;
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³ and R⁴ together with the carbon atoms to which they are attached form an optionally substituted fused carbocyclic or heterocyclic ring having from 5 to 7 ring members of which up to 3 can be heteroatoms selected from N, O and S; and
R⁵ is hydrogen, a group R² or a group R¹⁰ wherein R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
   and salts, N-oxides and solvates thereof.

The invention also provides:
The use of a compound of the formula (I) as defined herein for the manufacture of a medicament for the prophylaxis or treatment of a disease state or condition mediated by a cyclin dependent kinase or glycogen synthase kinase-3.
A method for the prophylaxis or treatment of a disease state or condition mediated by a cyclin dependent kinase or glycogen synthase kinase-3, which method comprises administering to a subject in need thereof a compound of the formula (I) as defined herein.
A method for alleviating or reducing the incidence of a disease state or condition mediated by a cyclin dependent kinase or glycogen synthase kinase-3, which method comprises administering to a subject in need thereof a compound of the formula (I) as defined herein.
A method for treating a disease or condition comprising or arising from abnormal cell growth in a mammal, which method comprises administering to the mammal a compound of the formula (I) as defined herein in an amount effective in inhibiting abnormal cell growth.
A method for alleviating or reducing the incidence of a disease or condition comprising or arising from abnormal cell growth in a mammal, which method comprises administering to the mammal a compound of the formula (I) as defined herein in an amount effective in inhibiting abnormal cell growth.
A method for treating a disease or condition comprising or arising from abnormal cell growth in a mammal, the method comprising administering to the mammal a compound of the formula (I) as defined herein in an amount effective to inhibit a cdk kinase (such as cdk1 or cdk2) or glycogen synthase kinase-3 activity.
A method for alleviating or reducing the incidence of a disease or condition comprising or arising from abnormal cell growth in a mammal, the method comprising administering to the mammal a compound of the formula (I) as defined herein in an amount effective to inhibit a cdk kinase (such as cdk1 or cdk2) or glycogen synthase kinase-3 activity.
A method of inhibiting a cyclin dependent kinase or glycogen synthase kinase-3, which method comprises contacting the kinase with a kinase-inhibiting compound of the formula (I) as defined herein.
A method of modulating a cellular process (for example cell division) by inhibiting the activity of a cyclin dependent kinase or glycogen synthase kinase-3 using a compound of the formula (I) as defined herein.
The use of a compound of the formula (I) as defined herein for the manufacture of a medicament for prophylaxis or treatment of a disease or condition characterised by up-regulation of an Aurora kinase (e.g. Aurora A kinase or Aurora B kinase).
The use of a compound of the formula (I) as defined herein for the manufacture of a medicament for the prophylaxis or treatment of a cancer, the cancer being one which is characterised by up-regulation of an Aurora kinase (e.g. Aurora A kinase or Aurora B kinase).
The use of a compound of the formula (I) as defined herein for the manufacture of a medicament for the prophylaxis or treatment of cancer in a patient selected from a sub-population possessing the Ile31 variant of the Aurora A gene.
The use of a compound of the formula (I) as defined herein for the manufacture of a medicament for the prophylaxis or treatment of cancer in a patient who has been diagnosed as forming part of a sub-population possessing the Ile31 variant of the Aurora A gene.
A method for the prophylaxis or treatment of a disease or condition characterised by up-regulation of an Aurora kinase (e.g. Aurora A kinase or Aurora B kinase), the method comprising administering a compound of the formula (I) as defined herein.
A method for alleviating or reducing the incidence of a disease or condition characterised by up-regulation of an Aurora kinase (e.g. Aurora A kinase or Aurora B kinase), the method comprising administering a compound of the formula (I) as defined herein.
A method for the prophylaxis or treatment of (or alleviating or reducing the incidence of) cancer in a patient suffering from or suspected of suffering from cancer; which method comprises (i) subjecting a patient to a diagnostic test to determine whether the patient possesses the Ile31 variant of the Aurora A gene; and (ii) where the patient does possess the said variant, thereafter administering to the patient a compound of the formula (I) as defined herein having Aurora kinase inhibiting activity.
A method for the prophylaxis or treatment of (or alleviating or reducing the incidence of) a disease state or condition characterised by up-regulation of an Aurora kinase (e.g. Aurora A kinase or Aurora B kinase); which method comprises (i) subjecting a patient to a diagnostic test to detect a marker characteristic of up-regulation of the Aurora kinase and (ii) where the diagnostic test is indicative of up-regulation of Aurora kinase, thereafter administering to the patient a compound of the formula (I) as defined herein having Aurora kinase inhibiting activity.
A compound of the formula (I) for use in medicine.
The use of a compound of the formula (I) as defined herein for the manufacture of a medicament for the prophylaxis or treatment of a disease state as described herein.
A compound of the formula (I) as defined herein for use in the prophylaxis or treatment of a disease state as described herein.
A compound as defined herein for any of the uses and methods set forth above, and as described elsewhere herein.
A compound of formula (I) or a salt (e.g. an acid addition salt), solvate, tautomer or N-oxide thereof for use in the treatment of B-cell lymphoma.
A compound of formula (I) or a salt (e.g. an acid addition salt), solvate, tautomer or N-oxide thereof for use in the treatment of chronic lymphocytic leukaemia.
A compound of formula (I) or a salt (e.g. an acid addition salt), solvate, tautomer or N-oxide thereof for use in the treatment of diffuse large B cell lymphoma.
A method of treatment of B-cell lymphoma, diffuse large B cell lymphoma or chronic lymphocytic leukaemia by administering to a patient in need of such treatment a compound of formula (I) or a salt (e.g. an acid addition salt), solvate, tautomer or N-oxide thereof.
A compound of formula (I) or a salt (e.g. an acid addition salt), solvate, tautomer or N-oxide thereof for use in the treatment of leukaemia in particular relapsed or refractory acute myelogenous leukemia, myelodysplastic syndrome, acute lymphocytic leukemia and chronic myelogenous leukemia.

The aforementioned methods and uses, and any other therapeutic and diagnostic methods and uses, and methods of treating animals and plants defined herein, may also employ any sub-group, sub-genus, preference or example falling within formula (I), for example the compounds of formulae (II) to (IXa) and any sub-groups thereof, uless the context indicates otherwise.

### General Preferences and Definitions

The following general preferences and definitions shall apply to each of the moieties R¹ to R¹⁰, and their various sub-groups, sub-definitions, examples and embodiments unless the context indicates otherwise. In this specification, a superscript letter following the number of an R group indicates that the R group is a sub-group of the R group designated solely by the number. Thus, for example R^{1a}, R^{1b} and R^{1c} are all sub groups of R¹, and, analogously, R^{9a} and R^{9b} are subgroups of R⁹. Thus, unless indicated otherwise, the general preferences, definitions and examples set out for, e.g. R¹ apply also to its sub-groups R^{1a}, R^{1b} R^{1c} etcetera, and similarly with the other R groups.

Any references to formula (I) herein shall also be taken to refer to formulae (II) to (IXa) and any other sub-group of compounds within formula (I) unless the context requires otherwise.

References to “compounds of the invention” as used herein refer not only to formula (I) but also to any sub-group, sub-genus, preference or example falling within formula (I), for example the compounds of formulae (II) to (IXa) and any sub-groups thereof.

The term upregulation of Aurora kinase as used herein is defined as including elevated expression or over-expression of Aurora kinase, including gene amplification (i.e. multiple gene copies) and increased expression by a transcriptional effect, and hyperactivity and activation of Aurora kinase, including activation by mutations.

References to “carbocyclic” and “heterocyclic” groups as used herein shall, unless the context indicates otherwise, include both aromatic and non-aromatic ring systems. Thus, for example, the term “carbocyclic and heterocyclic groups” includes within its scope aromatic, non-aromatic, unsaturated, partially saturated and fully saturated carbocyclic and heterocyclic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term “non-aromatic group” embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cyclohexenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings, or two fused five membered rings. Each ring may contain up to about four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) an imidazole ring fused to a 5- or 6-membered ring containing 1 or 2 ring heteroatoms;
g) an oxazole ring fused to a 5- or 6-membered ring containing 1 or 2 ring heteroatoms;
h) an isoxazole ring fused to a 5- or 6-membered ring containing 1 or 2 ring heteroatoms;
i) a thiazole ring fused to a 5- or 6-membered ring containing 1 or 2 ring heteroatoms;
j) an isothiazole ring fused to a 5- or 6-membered ring containing 1 or 2 ring heteroatoms;
k) a thiophene ring fused to a 5- or 6-membered ring containing 1, 2 or 3 ring heteroatoms;
l) a furan ring fused to a 5- or 6-membered ring containing 1, 2 or 3 ring heteroatoms;
m) an oxazole ring fused to a 5- or 6-membered ring containing 1 or 2 ring heteroatoms;
n) an isoxazole ring fused to a 5- or 6-membered ring containing 1 or 2 ring heteroatoms;
o) a cyclohexyl ring fused to a 5- or 6-membered ring containing 1, 2 or 3 ring heteroatoms; and
p) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzfuran, benzthiophene, benzimidazole, benzoxazole, isobenzoxazole, benzisoxazole, benzthiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), triazolopyrimidine (e.g. [1,2,4]triazolo[1,5-a]pyrimidine), benzodioxole and pyrazolopyridine (e.g. pyrazolo[1,5-a]pyridine) groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur. The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic thioamides, cyclic thioesters, cyclic ureas (e.g. as in imidazolidin-2-one) cyclic ester moieties (e.g. as in butyrolactone), cyclic sulphones (e.g. as in sulpholane and sulpholene), cyclic sulphoxides, cyclic sulphonamides and combinations thereof (e.g. thiomorpholine).

Particular examples include morpholine, piperidine (e.g. 1-piperidinyl, 2-piperidinyl, 3-piperidinyl and 4-piperidinyl), piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofliran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine. In general, preferred non-aromatic heterocyclic groups include saturated groups such as piperidine, pyrrolidine, azetidine, morpholine, piperazine and N-alkyl piperazines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R¹ is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 11, or 10, or 9, or 8, or 7, or 6, or 5 non-hydrogen atoms.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

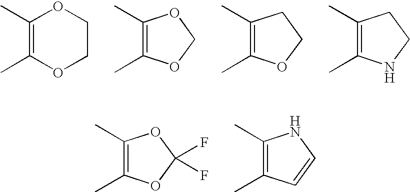

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone, except where otherwise stated. In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Preferred non-aromatic hydrocarbyl groups are saturated groups such as alkyl and cycloalkyl groups.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C¹, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members, more usually 3, 4, 5 or 6 ring members.

Where stated, one or more carbon atoms of a hydrocarbyl group may optionally be replaced by O, S, SO, SO₂, NR^{c}, X¹C(X²), C(X²)X¹ or X¹C(X²)X¹ wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}), and ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NRC^{c}(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S) NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O) NR^{c}, OC(S)NR^{c}, SC(S) NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO²NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups substituted by a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl and trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### Specific Embodiments of and Preferences for A, Q¹-Q⁴, R⁰ to R¹⁰ and X

In formula (I), each of Q¹ and Q² is a carbon atom; Q³ is selected from S and CH; and Q⁴ is selected from CR² and S; provided that one of Q³ and Q⁴ is S and the other of Q³ and Q⁴ is not S; and wherein when Q³ is S, there is a double bond between Q¹ and Q⁴ and a double bond between Q² and the adjacent ring nitrogen atom N; and when Q⁴ is S, there is a double bond between Q¹ and Q², and a double bond between Q³ and the adjacent ring nitrogen atom N.

In one general embodiment, Q³ is S and Q⁴ is CR² and hence the compound of the formula (I) is an isothiazole.

In another general embodiment, Q³ is CH and Q⁴ is S and hence the compound of the formula (I) is a thiazole.

In formula (I), X can be CR⁵ or N. In one particular embodiment, X is N. In another particular embodiment, X is CH. Preferably X is N.

R⁰ can be hydrogen or, together with the group R^{g} when present, can form a bridging group —(CH₂)ₚ— wherein p is 2 to 4, more usually 2-3, e.g. 2. Preferably R⁰ is hydrogen.

When R⁰ and the group R^{g} form a bridging group —(CH₂)ₚ—, the entity —(CH₂)ₘ—(B)ₙ—NR⁰— can be represented thus:

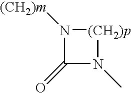

When A is a bond or a group —(CH₂)ₘ—(B)ₙ— wherein n is 0, X can be N or CR⁵ wherein R⁵ is hydrogen or a group R¹⁰. More preferably, X is N.

When A is a bond or a group —(CH₂)ₘ—(B)ₙ— wherein n is 1, it is preferred that X is N or CR⁵ wherein R⁵ is hydrogen or a group R². More preferably, X is N.

Where R⁵ is other than hydrogen, more particularly when n is 1, it is preferably a small substituent containing no more than 14 atoms, for example a C₁₋₄ alkyl or C₃₋₆ cycloalkyl group such as methyl, ethyl, propyl and butyl, or cyclopropyl and cyclobutyl.

A is a bond or —(CH₂)ₘ—(B)ₙ— wherein B is C═O, NR^{g}(C═O) or O(C═O), m is 0, 1 or 2; and n is 0 or 1. In one preferred group of compounds of the invention, m is 0 or 1, n is 1 and B is C═O or NR^{g}(C═O), preferably C═O. More preferably, m is 0, n is 1 and B is C═O. It is presently preferred that when B is NR^{g}(C═O), R^{g} is hydrogen.

It will be appreciated that the moiety R¹-A-NH linked to the moiety Q¹ can take the form of an amine R¹—(CH₂)ₘ—NH, an amide R¹—(CH₂)ₘ—C(═O)NH, a urea R¹—(CH₂)ₘ—NHC(═O)NH or a carbamate R¹—(CH₂)ₘ—OC(═O)NH wherein in each case m is 0, 1 or 2, preferably 0 or 1 and most preferably 0.

R¹ is hydrogen, a carbocyclic or heterocyclic group having from 3 to 12 ring members, or an optionally substituted C₁₋₈ hydrocarbyl group as hereinbefore defined. Examples of carbocyclic and heterocyclic, and optionally substituted hydrocarbyl groups are as set out above.

For example, R¹ can be a monocyclic or bicyclic group having from 3 to 10 ring members.

Where R¹ is a monocyclic group, typically it has 3 to 7 ring members, more usually 3 to 6 ring members, for example, 3, 4, 5 or 6.

When the monocyclic group R¹ is an aryl group, it will have 6 ring members and will be an unsubstituted or substituted phenyl ring.

When the monocyclic group R¹ is a non-aromatic carbocyclic group, it can have from 3 to 7 ring members, more usually 3 to 6 ring members, for example, 3, or 4, or 5, or 6 ring members. The non-aromatic carbocyclic group may be saturated or partially unsaturated but preferably it is saturated, i.e. R¹ is a cycloalkyl group.

When the monocyclic group R¹ is a heteroaryl group, it will have 5 or 6 ring members. Examples of heteroaryl groups having 5 and 6 ring members are set out above, and particular examples are described below.

In one sub-group of compounds, the heteroaryl group has 5 ring members.

In another sub-group of compounds, the heteroaryl group has 6 ring members.

The monocyclic heteroaryl groups R¹ typically have up to 4 ring heteroatoms selected from N, O and S, and more typically up to 3 ring heteroatoms, for example 1, or 2, or 3 ring heteroatoms.

When R¹ is a non-aromatic monocyclic heterocyclic group, it may be any one of the groups listed hereinabove or hereinafter. Such groups typically have from 4 to 7 ring members and more preferably 5 or 6 ring members. The non-aromatic monocyclic heterocyclic groups typically contain up to 3 ring heteroatoms, more usually 1 or 2 ring heteroatoms, selected from N, S and O. The heterocyclic group may be saturated or partially unsaturated, but preferably it is saturated. Particular examples of non-aromatic monocyclic heterocyclic groups are the particular and preferred examples defined in the “General Preferences and Definitions” section above, and as set out in the tables and examples below.

Where R¹ is a bicyclic group, typically it has 8 to 10 ring members, for example 8, or 9, or 10 ring members. The bicyclic group can be an aryl or heteroaryl group and examples of such groups include groups comprising a 5-membered ring fused to another 5-membered ring; a 5-membered ring fused to a 6-membered ring; and a 6-membered ring fused to another 6-membered ring. Examples of groups in each of these categories are set out above in the “General Preferences and Definitions” section.

A bicyclic aryl or heteroaryl group can comprise two aromatic or unsaturated rings, or one aromatic and one non-aromatic (e.g. partially saturated) ring.

Bicyclic heteroaryl groups typically contain up to 4 heteroatom ring members selected from N, S and O. Thus, for example, they may contain 1, or 2, or 3, or 4 heteroatom ring members.

In the monocyclic and bicyclic heterocyclic groups R¹, examples of combinations of heteroatom ring members include N; NN; NNN; NNNN; NO; NNO; NS, NNS, O, S, OO and SS.

Particular examples of R¹ include optionally substituted or unsubstituted heteroaryl groups selected from pyrazolo[1,5-a]pyridinyl (e.g. pyrazolo[1,5-a]pyridin-3-yl), furanyl (e.g. 2-furanyl and 3-furanyl), indolyl (e.g. 3-indolyl, 4-indolyl and 7-indolyl), oxazolyl, thiazolyl (e.g. thiazol-2-yl and thiazol-5-yl), isoxazolyl (e.g. isoxazol-3-yl and isoxazol-4-yl), pyrrolyl (e.g. 3-pyrrolyl), pyridyl (e.g. 2-pyridyl), quinolinyl (e.g. quinolin-8-yl), 2,3-dihydro-benzo[1,4]dioxine (e.g. 2,3-dihydro-benzo[1,4]dioxin-5-yl), benzo[1,3]dioxole (e.g. benzo[1,3]dioxol-4-yl), 2,3-dihydrobenzofuranyl (e.g. 2,3-dihydrobenzofuran-7-yl), imidazolyl and thiophenyl (e.g. 3-thiophenyl).

Other examples of R¹ include substituted or unsubsituted heteroaryl groups selected from pyrazolo [1,5-a]pyrimidine, isobenzofuran, [1,2,4]triazolo[1,5-a]pyrimidine, tetrazolyl, tetrahydroisoquinolinyl (e.g. 1,2,3,4-tetrahydroisoquinolin-7-yl), pyrimidinyl, pyrazolyl, triazolyl, 4,5,6,7-tetrahydro-benzo[d]isoxazole, phthalazine, 2H-phthalazin-1-one, benzoxazole, cinnoline, quinoxaline, naphthalene, benzo[c]isoxazole, imidazo[2,1-b]thiazole, pyridone, tetrahydroquinolinyl (e.g. 1,2,3,4-tetrahydroquinolin-6-yl), and 4,5,6,7-tetrahydro-benzofuran groups.

Preferred R¹ heteroaryl groups include pyrazolo[1,5-a]pyridinyl, furanyl, 2,3-dihydrobenzofuranyl, thiophenyl, indolyl, thiazolyl, isoxazolyl and 2,3-dihydro-benzo[1,4]dioxine groups.

Preferred aryl groups R¹ are optionally substituted phenyl groups.

Examples of non-aromatic groups R¹ include monocyclic cycloalkyl and azacycloalkyl groups such as cyclohexyl, cyclopentyl and piperidinyl, particularly cyclohexyl and 4-piperidinyl groups. Other examples of non-aromatic groups R¹ include monocyclic oxacycloalkyl groups such as tetrahydropyranyl and aza-oxa cycloalkyl groups such as morpholino (e.g. 2-morpholino and 4-morpholino).

Preferred substituted and unsubstituted C₁₋₈ hydrocarbyl groups include trifluoromethyl and tertiary butyl groups.

One sub-set of preferred R¹ groups includes phenyl, pyrazolo[1,5-a]pyridinyl and 2,3-dihydro-benzo[1,4]dioxine groups.

Another sub-set of preferred R¹ groups includes unsubstituted and substituted phenyl, pyrazolo[1,5-a]pyridinyl, 2,3-dihydro-benzo[1,4]dioxine, indol-4-yl, 2,3-dihydrobenzofuranyl, tert-butyl, furanyl, pyrazolo[1,5-a]pyridin-3-yl, pyrazolo[1,5-a]pyrimidin-3-yl, oxazolyl, isoxazolyl, benzoxazol-2-yl, 2H-tetrazol-5-yl, pyrazin-2-yl, pyrazolyl, benzyl, α,α-dimethylbenzyl, α-aminobenzyl, α-methylaminobenzyl, 4,5,6,7-tetrahydro-benzo[d]isoxazol-3-yl, 2H-phthalazin-1-one-4-yl, benzoxazol-7-yl, quinazolinyl, 2-naphthyl, cyclopropyl, benzo[c]isoxazol-3-yl, 4-piperidinyl, 5-thiazolyl, 2-pyridyl, 3-pyridyl, 3-pyrrolyl, isoxazolyl, imidazo[2,1-b]thiazolyl, 4-pyrimidinyl, cyclohexyl, tetrahydropyran-4-yl, tetrahydroquinolinyl, 4,5,6,7-tetrahydro-benzofuranyl and morpholinyl groups.

The group R¹ can be an unsubstituted or substituted carbocyclic or heterocyclic group in which one or more substituents can be selected from the group R¹⁰ as hereinbefore defined. In one embodiment, the substituents on R¹ may be selected from the group R^{10a} consisting of halogen, hydroxy, trifluoromethyl, cyano, nitro, carboxy, heterocyclic groups having 5 or 6 ring members and up to 2 heteroatoms selected from O, N and S, a group R^{a}-R^{b} wherein R^{a} is a bond, O, CO, X³C(X⁴), C(X⁴)X³, X³C(X⁴)X³, S, SO, or SO₂, and R_{b} is selected from hydrogen, heterocyclic groups having 5 or 6 ring members and up to 2 heteroatoms selected from O, N and S, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having 5 or 6 ring members and up to 2 heteroatoms selected from O, N and S; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

In a further embodiment, the substituents on R¹ may be selected from the group R^{10b} consisting of halogen, hydroxy, trifluoromethyl, cyano, nitro, carboxy, a group R^{a}-R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

In another embodiment, the substituents on R¹ may be selected from halogen, hydroxy, trifluoromethyl, a group R^{a}-R^{b} wherein R^{a} is a bond or O, and R^{b} is selected from hydrogen and a C₁₋₄ hydrocarbyl group optionally substituted by one or more substituents selected from hydroxyl and halogen.

One sub-set of substituents that may be present on a group R¹ (e.g. an aryl or heteroaryl group R¹) includes fluorine, chlorine, methoxy, methyl, oxazolyl, morpholino, trifluoromethyl, bromomethyl, chloroethyl, pyrrolidino, pyrrolidinylethoxy, pyrrolidinylmethyl, difluoromethoxy and morpholinomethyl.

Another sub-set of substituents that may be present on a group R¹ includes fluorine, chlorine, methoxy, ethoxy, methyl, ethyl, isopropyl, tert-butyl, amino, oxazolyl, morpholino, trifluoromethyl, bromomethyl, chloroethyl, pyrrolidino, pyrrolidinylethoxy, pyrrolidinylmethyl, difluoromethoxy, trifluoromethoxy, morpholino, N-methylpiperazino, piperazine, piperidino, pyrrolidino, and morpholinomethyl.

The moiety R¹ may be substituted by more than one substituent. Thus, for example, there may be 1 or 2 or 3 or 4 substituents, more typically 1, 2 or 3 substituents. In one embodiment, where R¹ is a six membered ring (e.g. a carbocyclic ring such as a phenyl ring), there may be a single substituent which may be located at any one of the 2-, 3- and 4-positions on the ring. In another embodiment, there may be two or three substituents and these may be located at the 2-, 3-, 4- or 6-positions around the ring. By way of example, a phenyl group R¹ may be 2,6-disubstituted, 2,3-disubstituted, 2,4-disubstituted 2,5-disubstituted, 2,3,6-trisubstituted or 2,4,6-trisubstituted.

In one embodiment, a phenyl group R¹ may be disubstituted at positions 2- and 6-with substituents selected from fluorine, chlorine and R^{a}-R^{b}, where R^{a} is O and R^{b} is C₁₋₄ alkyl, with fluorine being a particular substituent.

In one subgroup of compounds, the group R¹ is a five membered heteroaryl group containing 1 or 2 ring heteroatoms selected from O, N and S. Particular heteroaryl groups include furan, thiophene, pyrrole, oxazole, isoxazole and thiazole groups. The heteroaryl groups may be unsubstituted or substituted by one or more substituent groups as hereinbefore defined.

One preferred group of five membered heteroaryl groups consists of optionally substituted isoxazole and thiazole groups.

In another sub-group of compounds, R¹ is a pyrazolopyridine group, for example, a pyrazolo[1,5-a]pyridine group, such as a 3-pyrazolo[1,5-a]pyridinyl group.

Particular examples of groups R¹ include the groups A1 to A183 (e.g. A1 to A60) set out in Table 1 below.

TABLE 1

| | |
|---|---|
| | A1 |
| 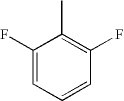 | |
| | A2 |
| 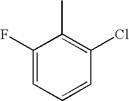 | |
| | A3 |
| 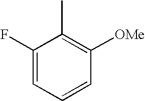 | |
| | A4 |
| 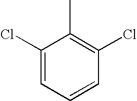 | |
| | A5 |
| 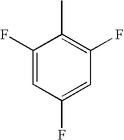 | |
| | A6 |
| 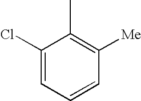 | |
| | A7 |
| 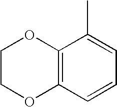 | |
| | A8 |
| 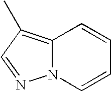 | |
| | A9 |
| 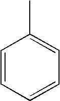 | |
| | A10 |
| 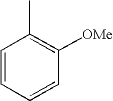 | |
| | A11 |
| 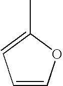 | |
| | A12 |
| 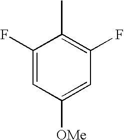 | |
| | A13 |
| 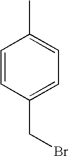 | |
| | A14 |
| 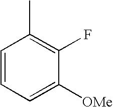 | |
| | A15 |
| 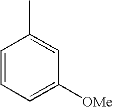 | |
| | A16 |
| 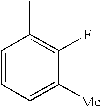 | |
| | A17 |
| 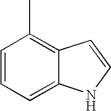 | |
| | A18 |
| 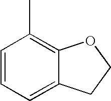 | |
| | A19 |
| 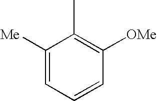 | |
| | A20 |
| 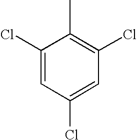 | |
| | A21 |
| 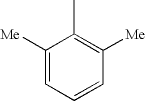 | |
| | A22 |
| 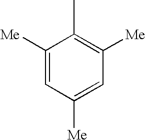 | |
| | A23 |
| 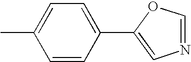 | |
| | A24 |
| 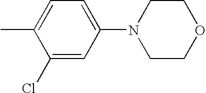 | |
| | A25 |
| 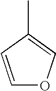 | |
| | A26 |
| 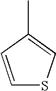 | |
| | A27 |
| 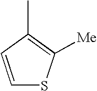 | |
| | A28 |
| 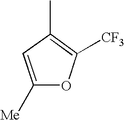 | |
| | A29 |
| 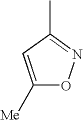 | |
| | A30 |
| 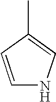 | |
| | A31 |
| 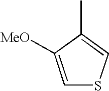 | |
| | A32 |
| 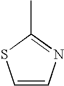 | |
| | A33 |
| 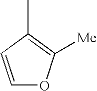 | |
| | A34 |
| 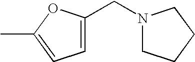 | |
| | A35 |
| 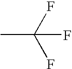 | |
| | A36 |
| 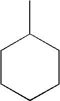 | |
| | A37 |
| 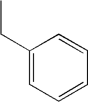 | |
| | A38 |
| 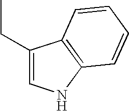 | |
| | A39 |
| 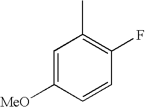 | |
| | A40 |
| 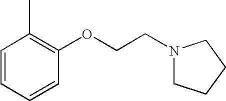 | |
| | A41 |
| 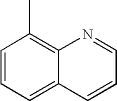 | |
| | A42 |
| 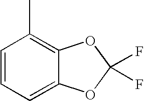 | |
| | A43 |
| 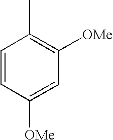 | |
| | A44 |
| 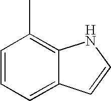 | |
| | A45 |
| 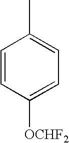 | |
| | A46 |
| 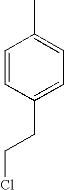 | |
| | A47 |
| 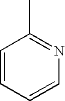 | |
| | A48 |
| 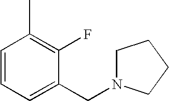 | |
| | A49 |
| 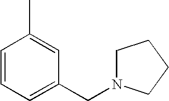 | |
| | A50 |
| 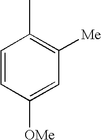 | |
| | A51 |
| 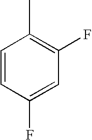 | |
| | A52 |
| 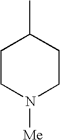 | |
| | A53 |
| 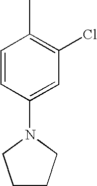 | |
| | A54 |
| 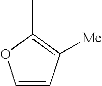 | |
| | A55 |
| 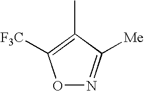 | |
| | A56 |
| 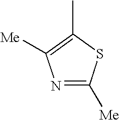 | |
| | A57 |
| 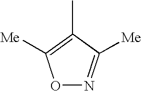 | |
| | A58 |
| 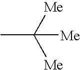 | |
| | A59 |
| 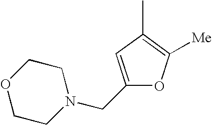 | |
| | A60 |
| 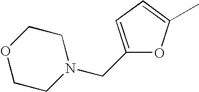 | |
| | A61 |
| 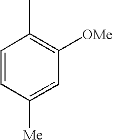 | |
| | A62 |
| 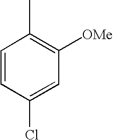 | |
| | A63 |
| 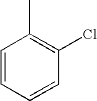 | |
| | A64 |
| 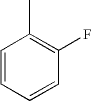 | |
| | A65 |
| 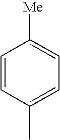 | |
| | A66 |
| 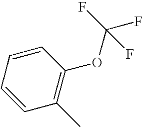 | |
| | A67 |
| 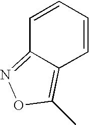 | |
| | A68 |
| 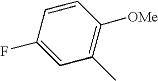 | |
| | A69 |
| 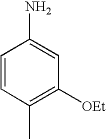 | |
| | A70 |
| 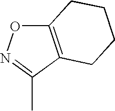 | |
| | A71 |
| 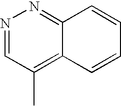 | |
| | A72 |
| 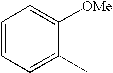 | |
| | A73 |
| 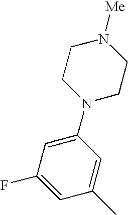 | |
| | A74 |
| 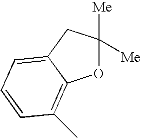 | |
| | A75 |
| 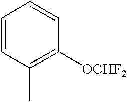 | |
| | A76 |
| 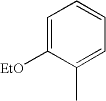 | |
| | A77 |
| 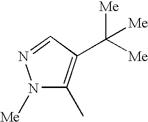 | |
| | A78 |
| 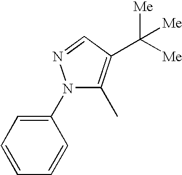 | |
| | A79 |
| 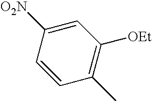 | |
| | A80 |
| 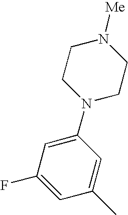 | |
| | A81 |
| 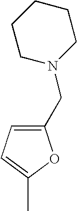 | |
| | A82 |
| 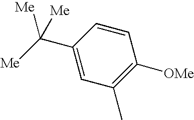 | |
| | A83 |
| 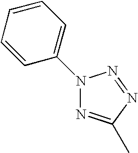 | |
| | A84 |
| 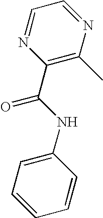 | |
| | A85 |
| 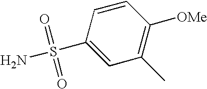 | |
| | A86 |
| 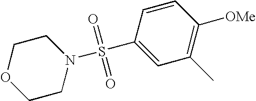 | |
| | A87 |
| 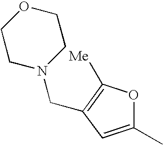 | |
| | A88 |
| 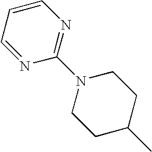 | |
| | A89 |
| 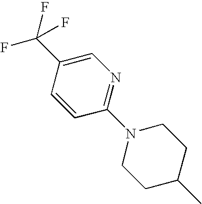 | |
| | A90 |
| 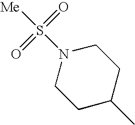 | |
| | A91 |
| 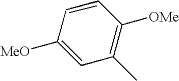 | |
| | A92 |
| 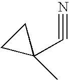 | |
| | A93 |
| 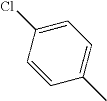 | |
| | A94 |
| 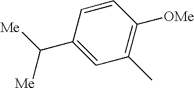 | |
| | A95 |
| 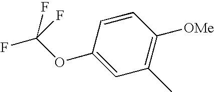 | |
| | A96 |
| 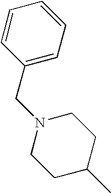 | |
| | A97 |
| 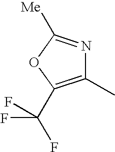 | |
| | A98 |
| 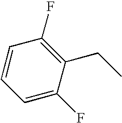 | |
| | A99 |
| 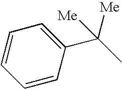 | |
| | A100 |
| 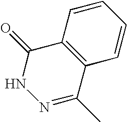 | |
| | A101 |
| 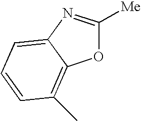 | |
| | A102 |
| 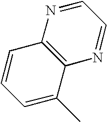 | |
| | A103 |
| 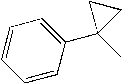 | |
| | A104 |
| 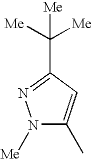 | |
| | A105 |
| 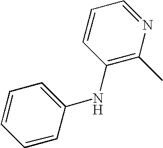 | |
| | A106 |
| 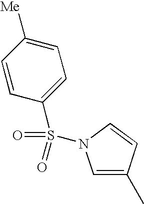 | |
| | A107 |
| 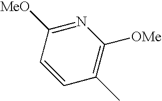 | |
| | A108 |
| 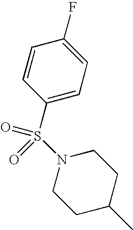 | |
| | A109 |
| 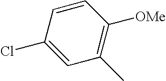 | |
| | A110 |
| 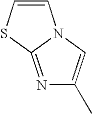 | |
| | A111 |
| 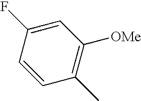 | |
| | A112 |
| 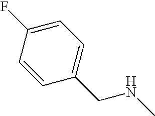 | |
| | A113 |
| 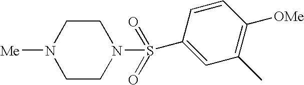 | |
| | A114 |
| 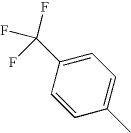 | |
| | A115 |
| 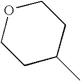 | |
| | A116 |
| 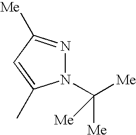 | |
| | A117 |
| 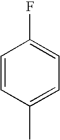 | |
| | A118 |
| 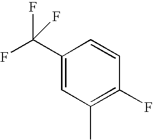 | |
| | A119 |
| 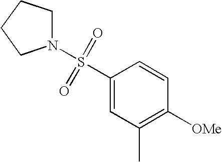 | |
| | A120 |
| 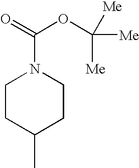 | |
| | A121 |
| 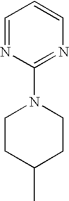 | |
| | A122 |
| 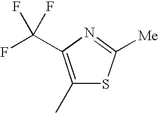 | |
| | A123 |
| 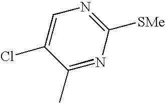 | |
| | A124 |
| 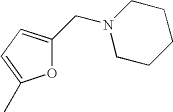 | |
| | A125 |
| 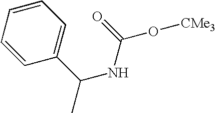 | |
| | A126 |
| 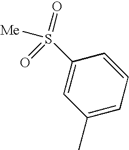 | |
| | A127 |
| 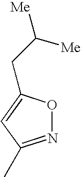 | |
| | A128 |
| 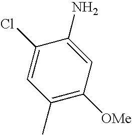 | |
| | A129 |
| 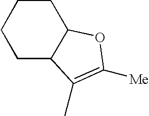 | |
| | A130 |
| 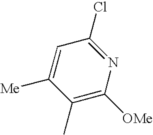 | |
| | A131 |
| 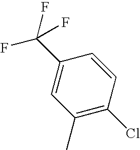 | |
| | A132 |
| 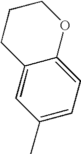 | |
| | A133 |
| 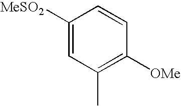 | |
| | A134 |
| 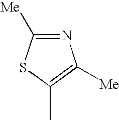 | |
| | A135 |
| 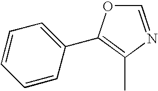 | |
| | A136 |
| 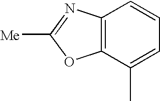 | |
| | A137 |
| 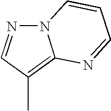 | |
| | A138 |
| 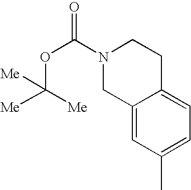 | |
| | A139 |
| 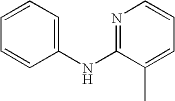 | |
| | A140 |
| 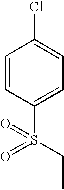 | |
| | A141 |
| 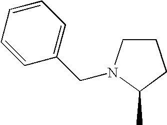 | |
| | A142 |
| 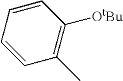 | |
| | A143 |
| 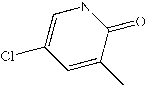 | |
| | A144 |
| 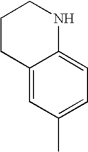 | |
| | A145 |
| 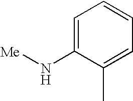 | |
| | A146 |
| 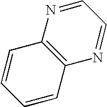 | |
| | A147 |
| 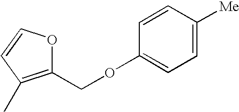 | |
| | A148 |
| 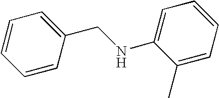 | |
| | A149 |
| 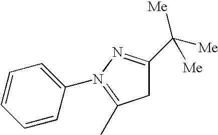 | |
| | A150 |
| 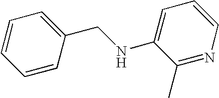 | |
| | A151 |
| 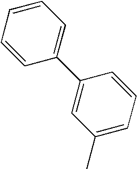 | |
| | A152 |
| 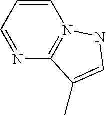 | |
| | A153 |
| 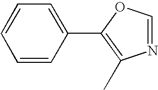 | |
| | A154 |
| 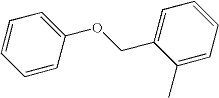 | |
| | A155 |
| 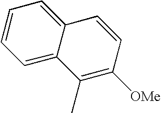 | |
| | A156 |
| 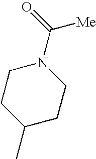 | |
| | A157 |
| 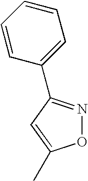 | |
| | A158 |
| 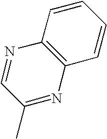 | |
| | A159 |
| 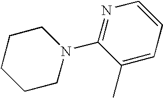 | |
| | A160 |
| 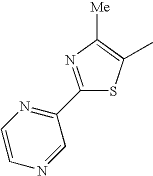 | |
| | A161 |
| 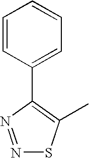 | |
| | A162 |
| 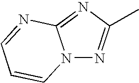 | |
| | A163 |
| 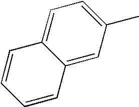 | |
| | A164 |
| 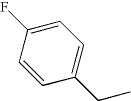 | |
| | A165 |
| 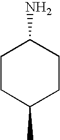 | |
| | A166 |
| 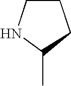 | |
| | A167 |
| 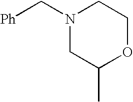 | |
| | A168 |
| 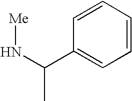 | |
| | A169 |
| 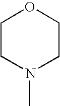 | |
| | A170 |
| 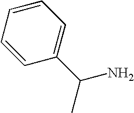 | |
| | A171 |
| 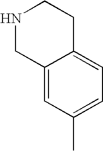 | |
| | A172 |
| 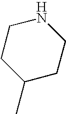 | |
| | A173 |
| 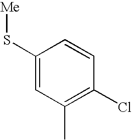 | |
| | A174 |
| 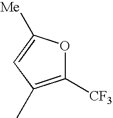 | |
| | A175 |
| 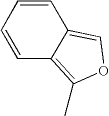 | |
| | A176 |
| 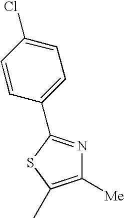 | |
| | A177 |
| 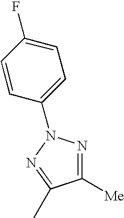 | |
| | A178 |
| 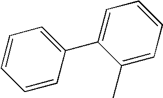 | |
| | A179 |
| 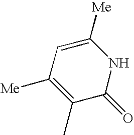 | |
| | A180 |
| 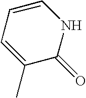 | |
| | A181 |
| 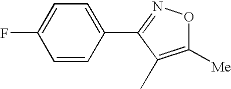 | |
| | A182 |
| 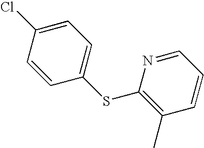 | |
| | A183 |
| 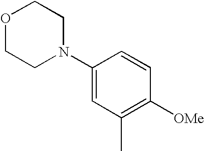 | |
| | A184 |
| 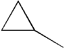 | |

One preferred sub-set of compounds of the invention is the sub-set wherein R¹ is a group selected from A1 to A34.

Another preferred sub-set of compounds of the invention is the sub-set wherein R¹ is a group selected from A1 to A24, A26 to A34, A38 to A46, A48 to A57, A59 to A64, A66 to A114, A116 to A165, A167 to A168 and A170 to A183.

Another preferred subset of compounds is the subset in which R¹ is a group A184.

One particularly preferred sub-set of groups R¹ includes 2,6-difluorophenyl, 2-chloro-6-fluorophenyl, 2-fluoro-6-methoxyphenyl, 2,6-dichlorophenyl, 2,4,6-trifluorophenyl, 2-chloro-6-methyl, 2,3-dihydro-benzo[1,4]dioxin-5-yl and pyrazolo[1,5-a]pyridin-3-yl. Compounds containing groups R¹ selected from this sub-set have particularly good cdk inhibitory activity.

Another particularly preferred sub-set of groups R¹ includes 2,6-difluorophenyl, 2-methoxyphenyl, 2,6-difluoro-4-methoxyphenyl, 2-fluoro-6-methoxyphenyl, 2-fluoro-5-methoxyphenyl, 2,6-dimethoxyphenyl, 2,4-dimethoxyphenyl, 2-chloro-6-fluorophenyl, 2,6-dichlorophenyl, 2,4,6-trifluorophenyl, 2-chloro-6-methyl, 2,3-dihydro-benzo[1,4]dioxin-5-yl and pyrazolo[1,5-a]pyridin-3-yl.

One currently preferred group R¹ is 2,6-difluorophenyl.

Another preferred group R¹ is cyclopropyl.

R² is hydrogen, halogen, methoxy, or a C₁₋₄ hydrocarbyl group optionally substituted by halogen, hydroxyl or methoxy. Preferably R² is hydrogen, chlorine or methyl, and most preferably R² is hydrogen.

In the compounds of the formula (I), R³ and R⁴, together with the carbon atoms to which they are attached, form a fused heterocyclic or carbocyclic group having from 5 to 7 ring members, of which up to 3 can be heteroatoms selected from N, O and S. The fused carbocyclic or heterocyclic ring can be optionally substituted by 0 to 4 groups R¹⁰ as defined herein. The fused heterocyclic or carbocyclic group can be aromatic or non-aromatic but preferably is aromatic.

In one preferred group of compounds, R³ and R⁴ together with the carbon atoms to which they are attached form a fused carbocyclic group having from 5 to 7 ring members.

Fused five and six membered carbocyclic or heterocyclic groups are particularly preferred. Examples of fused heterocyclic rings include five and six membered rings such as thiazolo, isothiazolo, oxazolo, isoxazolo, pyrrolo, pyrido, thieno, furano, pyrimido, pyrazolo, pyrazino, tetrahydroazepinone and imidazolo fused rings. It is preferred that the fused heterocyclic group is selected from six membered ring groups, one particularly preferred group being the pyrido group.

Examples of fused carbocyclic rings include five and six membered rings such as benzo, dihydro or tetrahydro-benzo and cyclopenta-fused rings. Six membered rings are preferred. One particularly preferred group is the benzo group.

Particular examples of ring systems formed by the five membered ring and R³ and R⁴ are ring systems (i) to (iv) set out below. Ring system (i) is generally preferred.

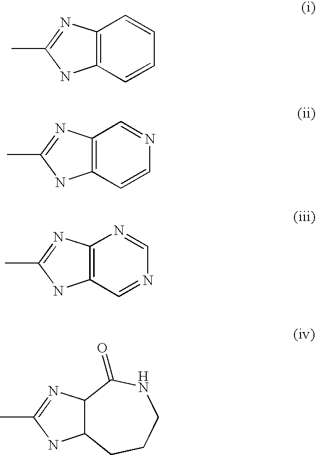

The fused carbocyclic or heterocyclic group can be optionally substituted by one or more groups R¹⁰ as hereinbefore defined.

In one embodiment, the substituents on the fused carbocyclic or heterocyclic group may be selected from halogen, hydroxy, trifluoromethyl, cyano, nitro, carboxy, amino, monocyclic carbocyclic and heterocyclic groups having from 3 to 7 (typically 5 or 6) ring members, a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{C}SO₂; and R^{b} is selected from hydrogen, a carbocyclic or heterocyclic group with 3-7 ring members and a C₁₋₈ hydrocarbyl group optionally substituted by one or more substituents selected from hydroxy, oxo, halogen, cyano, nitro, carboxy, amino, mono- or di-C₁₋₄ hydrocarbylamino, a carbocyclic or heterocyclic group with 3-7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; and R^{c}, X¹ and X² are as hereinbefore defined, or two adjacent groups R¹⁰ together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S.

Preferred R¹⁰ groups on the fused carbocyclic or heterocyclic group formed by R³ and R⁴ include halogen (e.g.fluorine and chlorine), a group R^{a}-R^{b} wherein R^{a} is a bond, O, CO, C(X²)X¹, and R^{b} is selected from hydrogen, heterocyclic groups having 3-7 ring members (preferably 5 or 6 ring mbers) and a C₁₋₄ hydrocarbyl group (e.g. a saturated hydrocarbyl group such as an alkyl or cycloalkyl group) optionally substituted by one or more substituents selected from hydroxy, carboxy, amino, mono- or di-C₁₋₄ hydrocarbylamino, and heterocyclic groups with 3-7 ring members (e.g. 5 or 6 ring members).

One preferred group of compounds of the invention is represented by the formula (II):

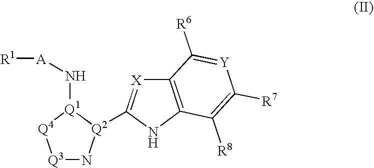

wherein Q¹-Q⁴, R¹, R² and X are as defined herein;

Y is N or CR⁹ wherein R⁹ is hydrogen or a group R¹⁰; and

R⁶, R⁷ and R⁸ are the same or different and each is hydrogen or a group R¹⁰ as defined herein.

In one sub-group of compounds of the formula (II), X is N.

In another sub-group of compounds of the formula (II), Y is CR⁹.

When Y is N, it is preferred that R⁶ is other than amino.

In one embodiment, the compounds of the invention are represented by the formula (III):

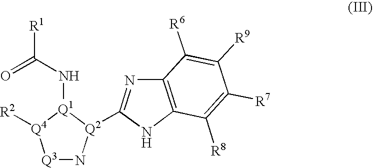

wherein Q¹-Q⁴, R¹, R² and R⁶ to R⁹ are as defined herein.

Another embodiment of the invention can be represented by the formula (IIIa):

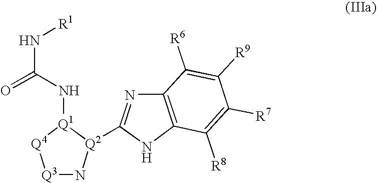

Within formula (III) and formula (IIIa), it is preferred that R² is hydrogen or C₁₋₄ alkyl, and more typically R² is hydrogen.

Within the group of compounds defined by the formula (III), R¹ is preferably 2,3 disubstituted, 2,6 disubstituted or 2,4,6, trisubstituted phenyl or 2,3-dihydro-benzo[1,4]dioxine, where the substituents are selected from halogen and C₁₋₄ alkoxy.

More preferably R¹ is selected from 2,6-difluorophenyl, 2-fluoro-6-methoxyphenyl, 2-chloro-6-fluorophenyl, 2,6-dichlorophenyl, 2,4,6-trifluorophenyl, 2,6-difluoro-4-methoxyphenyl, and 2,3-dihydro-benzo[1,4]dioxine.

One particularly preferred group R¹ is 2,6-difluorophenyl.

Another particularly preferred group R¹ is cyclopropyl.

The moieties R⁶, R⁷, R⁸ and R⁹ are typically selected from hydrogen, halogen, hydroxy, trifluoromethyl, cyano, nitro, carboxy, amino, monocyclic carbocyclic and heterocyclic groups having from 3 to 12 (preferably 3 to 7, and more typically 5 or 6) ring members, a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, a carbocyclic or heterocyclic group with 3-7 ring members and a C₁₋₈ hydrocarbyl group optionally substituted by one or more substituents selected from hydroxy, C₁₋₄ acyloxy, oxo, halogen, cyano, nitro, carboxy, amino, mono- or di-C₁₋₄ hydrocarbylamino, a carbocyclic or heterocyclic group with 3-7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; and R^{c}, X¹ and X²; or an adjacent pair of substituents selected from R⁶, R⁷, R⁸ and R⁹ together with the carbon atoms to which they are attached may form a non-aromatic five or six membered ring containing up to three heteroatoms selected from O, N and S.

In one embodiment, R⁶ to R⁹ are each hydrogen or are selected from halogen, cyano, hydroxy, trifluoromethyl, nitro, a group R^{a}-R^{b} wherein R^{a} is a bond, O, CO or C(X²)X¹ and R^{b} is selected from hydrogen, heterocyclic groups having from 3 to 12 ring members (preferably 4 to 7 ring members, e.g. 5 and 6 ring members), and a C₁₋₈ hydrocarbyl group (preferably a C₁₋₄ hydrocarbyl group, e.g. a saturated hydrocarbyl group such as alkyl or cyclopropyl), optionally substituted by one or more substituents selected from hydroxy, C₁₋₄ acyloxy, mono- or di-C₁₋₄ hydrocarbylamino (e.g. monoalkylamino and dialkylamino), heterocyclic groups having from 3 to 12 ring members, more preferably 4 to 7 ring members (e.g. 5 or 6 ring members); where R^{c} is selected from hydrogen and C₁₋₄ hydrocarbyl (e.g. saturated hydrocarbyl such as alkyl and cycloalkyl), X¹ is O or NR^{c} and X² is ═O.

In another embodiment, R⁶, R⁷, R⁸ and R⁹ are selected from hydrogen, fluorine, chlorine, bromine, nitro, trifluoromethyl, carboxy, a group R^{a}-R^{b} wherein R^{a} is a bond, O, CO, C(X²)X¹, and R^{b} is selected from hydrogen, heterocyclic groups having 3-7 ring members (e.g. pyrrolidine, N-methyl piperazine or morpholine) and a C₁₋₄ hydrocarbyl group optionally substituted by one or more substituents selected from hydroxy, carboxy, C₁₋₄ acyloxy, amino, mono- or di-C₁₋₄ hydrocarbylamino, heterocyclic groups with 3-7 ring members (e.g. pyrrolidine, N-methyl piperazine or morpholine); or an adjacent pair of substituents selected from R⁶, R⁷, R⁸ and R⁹ together with the carbon atoms to which they are attached may form a non-aromatic five or six membered ring containing one or two oxygen atoms as ring members.

In a more preferred embodiment, R⁶, R⁷, R⁸ and R⁹ are selected from hydrogen, fluorine, chlorine, trifluoromethyl, a group R^{a}-R^{b} wherein R^{a} is a bond, O, CO, C(X²)X¹, and R^{b} is selected from hydrogen, saturated heterocyclic groups having 5-6 ring members and a C₁₋₂ hydrocarbyl group optionally substituted by one or more substituents selected from hydroxy, carboxy, C₁₋₂ acyloxy, amino, mono- or di-C₁₋₄ hydrocarbylamino, heterocyclic groups with 5-6 ring members; or an adjacent pair of substituents selected from R⁶, R⁷, R⁸ and R⁹ may form a methylenedioxy or ethylenedioxy group each optionally substituted by one or more fluorine atoms.

In another embodiment, particular substituent groups R⁶ to R⁹ include halogen, nitro, carboxy, a group R^{a}-R^{b} wherein R^{a} is a bond, O, CO, C(X²)X¹, and R^{b} is selected from hydrogen, heterocyclic group having 3-7 ring members and a C₁₋₄ hydrocarbyl group optionally substituted by one or more substituents selected from hydroxy, carboxy, amino, mono- or di-C₁₋₄ hydrocarbylamino, heterocyclic group with 3-7 ring members.

Whereas each of R⁶ to R⁹ can be hydrogen or a substituent as hereinbefore defined, it is preferred that at least one, more preferably at least two, of R⁶ to R⁹ are hydrogen.

In one particular embodiment, one of R⁶ to R⁹ is a substituent and the others each are hydrogen. For example, R⁶ can be a substituent group and R⁷ to R⁹ can each be hydrogen, or R⁹ can be a substituent and R⁶, R⁷ and R⁸ can each be hydrogen.

In another particular embodiment, two of R⁶ to R⁹ are substituents and the other two are both hydrogen. For example, R⁶ and R⁹ can both be substituents when R⁷ and R⁸ are both hydrogen; or R⁶ and R⁷ can both be substituents when R⁸ and R⁹ are both hydrogen; or R⁷ and R⁹ can both be substituents when R⁶ and R⁸ are both hydrogen.

R⁶ is preferably selected from:

hydrogen;

halogen (preferably fluorine or chlorine);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R⁷ is preferably selected from:

hydrogen;

halogen (preferably fluorine or chlorine);

C₁₋₄ alkoxy (for example methoxy);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R⁸ is preferably selected from hydrogen, fluorine and methyl, most preferably hydrogen.

R⁹ is preferably selected from:

hydrogen;

halogen (preferably fluorine or chlorine);

C₁₋₄ alkoxy (for example methoxy);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

Alternatively, R⁶ and R⁹, or R⁷ and R⁹, together with the carbon atoms to which they are attached may form a cyclic group selected from:

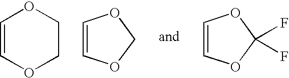

In the foregoing definitions, when R¹¹ and R¹² together with the nitrogen atom in the group NR¹¹R¹² form a five or six membered heterocyclic ring, the heteroatom ring members are preferably selected from O and N. The heterocyclic ring is typically non-aromatic and examples of such rings include morpholine, piperazine, N—C₁₋₄-alkylpiperazine, piperidine and pyrrolidine. Particular examples of N—C₁₋₄-alkylpiperazine groups include N-methylpiperazine and N-isopropylpiperazine.

Preferred groups R⁶ to R⁹ include those in which the benzimidazole group

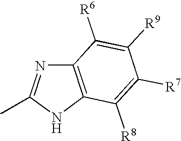

is as shown in Table 2 below.

TABLE 2

| | |
|---|---|
| | B1 |
| 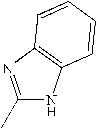 | |
| | B2 |
| 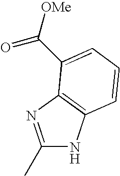 | |
| | B3 |
| 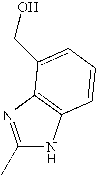 | |
| | B4 |
| 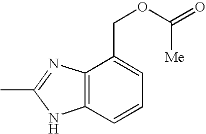 | |
| | B5 |
| 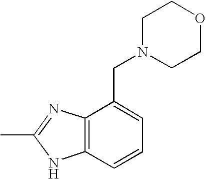 | |
| | B6 |
| 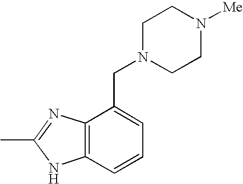 | |
| | B7 |
| 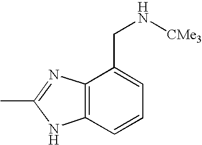 | |
| | B8 |
| 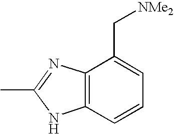 | |
| | B9 |
| 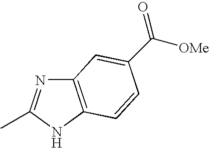 | |
| | B10 |
| 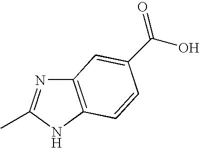 | |
| | B11 |
| 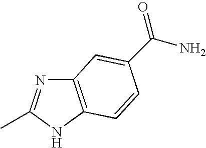 | |
| | B12 |
| 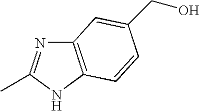 | |
| | B13 |
| 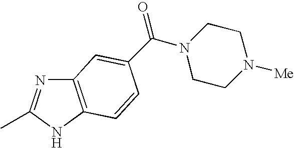 | |
| | B14 |
| 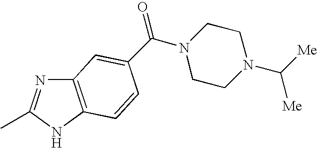 | |
| | B15 |
| 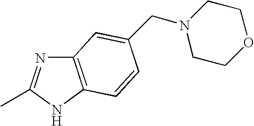 | |
| | B16 |
| 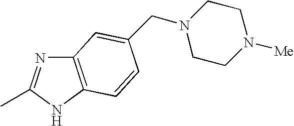 | |
| | B17 |
| 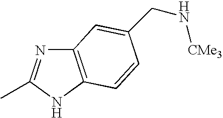 | |
| | B18 |
| 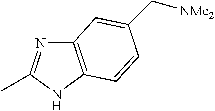 | |
| | B19 |
| 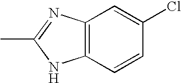 | |
| | B20 |
| 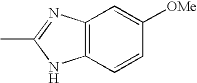 | |
| | B21 |
| 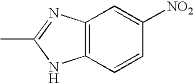 | |
| | B22 |
| 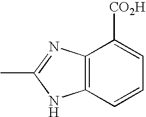 | |
| | B23 |
| 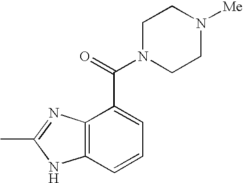 | |
| | B24 |
| 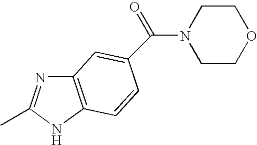 | |
| | B25 |
| 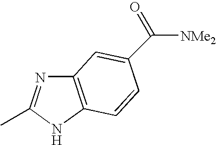 | |
| | B26 |
| 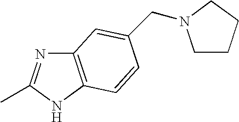 | |
| | B27 |
| 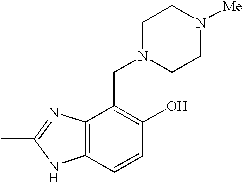 | |
| | B28 |
| 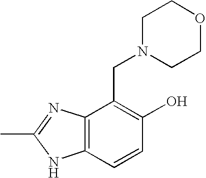 | |
| | B29 |
| 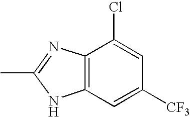 | |
| | B30 |
| 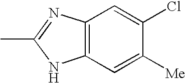 | |
| | B31 |
| 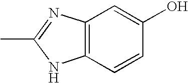 | |
| | B32 |
| 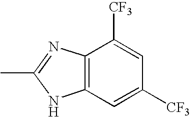 | |
| | B33 |
| 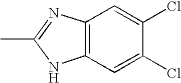 | |
| | B34 |
| 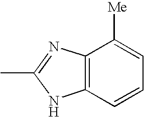 | |
| | B35 |
| 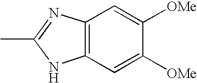 | |
| | B36 |
| 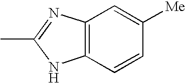 | |
| | B37 |
| 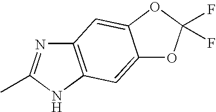 | |
| | B38 |
| 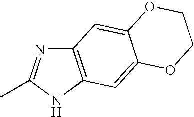 | |
| | B39 |
| 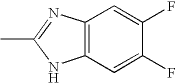 | |
| | B40 |
| 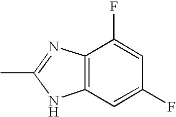 | |
| | B41 |
| 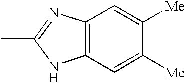 | |
| | B42 |
| 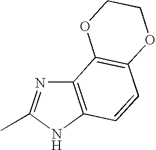 | |
| | B43 |
| 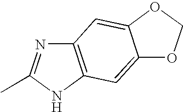 | |
| | B44 |
| 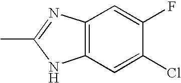 | |
| | B45 |
| 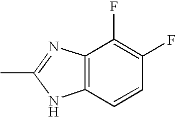 | |
| | B46 |
| 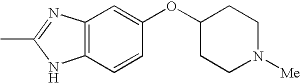 | |
| | B47 |
| 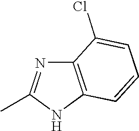 | |
| | B48 |
| 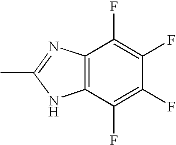 | |
| | B49 |
| 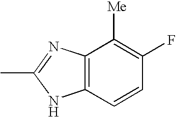 | |
| | B50 |
| 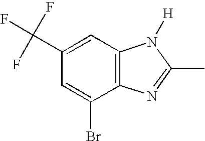 | |
| | B51 |
| 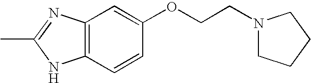 | |
| | B52 |
| 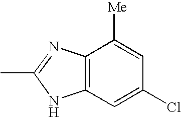 | |
| | B53 |
| 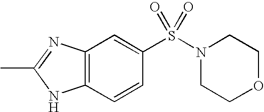 | |
| | B54 |
| 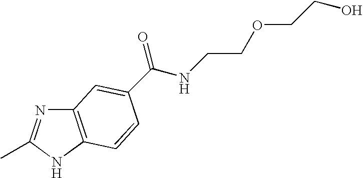 | |
| | B55 |
| 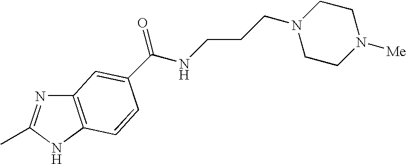 | |
| | B56 |
| 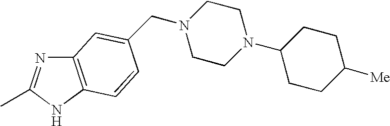 | |
| | B57 |
| 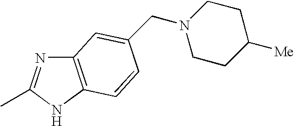 | |
| | B58 |
| 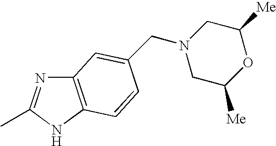 | |
| | B59 |
| 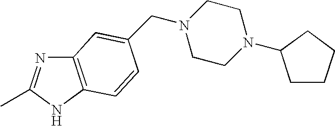 | |
| | B60 |
| 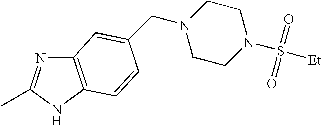 | |
| | B61 |
| 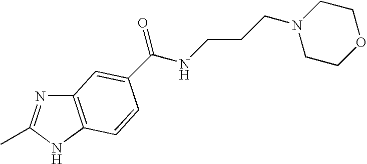 | |
| | B62 |
| 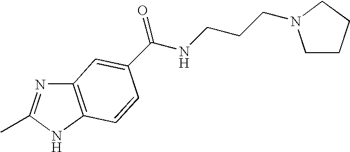 | |
| | B63 |
| 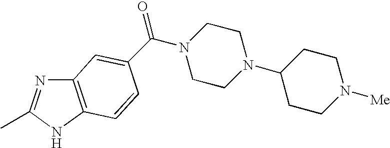 | |
| | B64 |
| 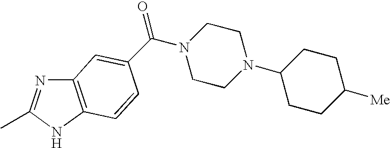 | |
| | B65 |
| 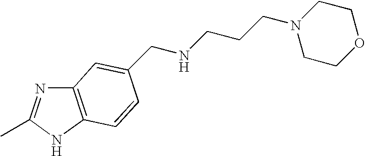 | |
| | B66 |
| 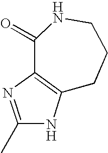 | |
| | B67 |
| 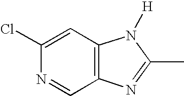 | |
| | B68 |
| 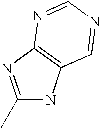 | |
| | B69 |
| 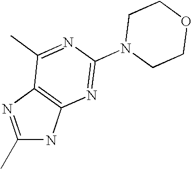 | |
| | B70 |
| 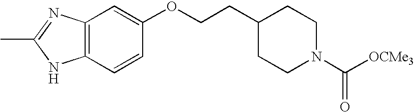 | |
| | B71 |
| 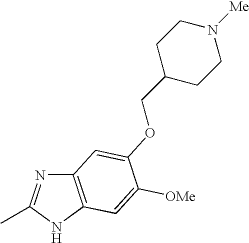 | |

Of the benzimidazole groups set out in Table 2 above, particular groups include groups B1, B3, B5-B8, B11-B20, B23-B30 and B32-B47.

One sub-set of preferred compounds is the group of compounds wherein the benzimidazole moiety is selected from groups B1, B3, B5-B8, B11-B20, B24, B25, B27-B30 and B32-B47.

Particularly preferred benzimidazole moieties are groups B8, B15 and B35, and more particularly group B15.

One group of novel compounds of the invention can be represented by the formula (IV):

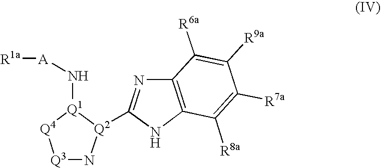

wherein A is NH(C═O), O(C═O) or C═O;

R^{6a}, R^{7a}, R^{8a} and R^{9a} are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X₂)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R^{6a}, R^{7a}, R^{8a} or R^{9a} together with the carbon atoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c}; or an adjacent pair of substituents selected from R^{6a}, R^{7a}, R^{8a} and R^{9a} together with the carbon atoms to which they are attached may form a non-aromatic five or six membered ring containing up to three heteroatoms selected from O, N and S; R^{1a} is selected from:
6-membered monocyclic aryl groups substituted by one to three substituents R^{10c} provided that when the aryl group is substituted by a methyl group, at least one substituent other than methyl is present;
6-membered monocyclic heteroaryl groups containing a single heteroatom ring member which is nitrogen, the heteroaryl groups being substituted by one to three substituents R^{10c};
5-membered monocyclic heteroaryl groups containing up to three heteroatom ring members selected from nitrogen and sulphur, and being optionally substituted by one to three substituents R^{10c};
5-membered monocyclic heteroaryl groups containing a single oxygen heteroatom ring member and optionally a nitrogen heteroatom ring member, and being substituted by one to three substituents R^{10c} provided that when the heteroaryl group contains a nitrogen ring member and is substituted by a methyl group, at least one substituent other than methyl is present;
bicyclic aryl and heteroaryl groups having up to four heteroatom ring members and wherein either one ring is aromatic and the other ring is non-aromatic, or wherein both rings are aromatic, the bicyclic groups being optionally substituted by one to three substituents R^{10c};
four-membered, six-membered and seven-membered monocyclic C-linked saturated heterocyclic groups containing up to three heteroatoms selected from nitrogen, oxygen and sulphur, the heterocyclic groups being optionally substituted by one to three substituents R^{10c} provided that when the heterocyclic group has six ring members and contains only one heteroatom which is oxygen, at least one substituent R^{10c} is present;
five membered monocyclic C-linked saturated heterocyclic groups containing up to three heteroatoms selected from nitrogen, oxygen and sulphur, the heterocyclic groups being optionally substituted by one to three substituents R^{10c} provided that when the heterocyclic group has five ring members and contains only one heteroatom which is nitrogen, at least one substituent R^{10c} other than hydroxy is present;
four and six membered cycloalkyl groups optionally substituted by one to three substituents R^{10c};
three and five membered cycloalkyl groups substituted by one to three substituents R^{10c}; and
a group Ph′CR¹⁷R¹⁸— where Ph′ is a phenyl group substituted by one to three substituents R^{10c}; R¹⁷ and R¹⁸ are the same or different and each is selected from hydrogen and methyl; or R¹⁷ and R¹⁸ together with the carbon atom to which they are attached form a cyclopropyl group; or one of R¹⁷ and R¹⁸ is hydrogen and the other is selected from amino, methylamino, C₁₋₄ acylamino, and C₁₋₄ alkoxycarbonylamino;

and where one of R^{6a}, R^{7a}, R^{8a} and R^{9a} is a morpholinomethyl group, then R^{1a} is additionally selected from:
unsubstituted phenyl and phenyl substituted with one or more methyl groups;
unsubstituted 6-membered monocyclic heteroaryl groups containing a single heteroatom ring member which is nitrogen;
unsubstituted furyl;
5-membered monocyclic heteroaryl groups containing a single oxygen heteroatom ring member and a nitrogen heteroatom ring member, and being unsubstituted or substituted by one or more methyl groups;
unsubstituted six membered monocyclic C-linked saturated heterocyclic groups containing only one heteroatom which is oxygen; and
unsubstituted three and five membered cycloalkyl groups;
   and R^{10c} is selected from:
halogen (e.g. F and Cl);
hydroxyl;
C₁₋₄ hydrocarbyloxy optionally substituted by one or more substituents selected from hydroxyl and halogen;
C₁₋₄ hydrocarbyl substituted by one or more substituents selected from hydroxyl, halogen and five and six-membered saturated heterocyclic rings containing one or two heteroatom ring members selected from nitrogen, oxygen and sulphur;
S—C₁₋₄ hydrocarbyl;
phenyl optionally substituted with one to three substituents selected from C₁₋₄ alkyl, trifluoromethyl, fluoro and chloro;
heteroaryl groups having 5 or 6 ring members (e.g. oxazole, pyridyl, pyrimidinyl) and containing up to 3 heteroatoms selected from N, O and S, the heteroaryl groups being optionally substituted with one to three substituents selected from C₁₋₄ alkyl, trifluoromethyl, fluoro and chloro;
5- and 6-membered non-aromatic heterocyclic groups (e.g. pyrrolidino, piperidino, piperazine, N-methylpiperazino, morpholino) containing up to 3 heteroatoms selected from N, O and S and being optionally substituted with one to three substituents selected from C₁₋₄ alkyl, trifluoromethyl, fluoro and chloro;
cyano, nitro, amino, C₁₋₄ alkylamino, di-C₁₋₄ alkylamino, C₁₋₄ acylamino, C¹⁻⁴ alkoxycarbonylamino;
a group R¹⁹—S(O)ₙ— where n is 0, 1 or 2 and R¹⁹ is selected from amino; C₁₋₄ alkylamino; di-C₁₋₄ alkylamino; C₁₋₄ hydrocarbyl; phenyl optionally substituted with one to three substituents selected from C₁₋₄ alkyl, trifluoromethyl, fluoro and chloro; and 5- and 6-membered non-aromatic heterocyclic groups containing up to 3 heteroatoms selected from N, O and S and being optionally substituted with one to three C₁₋₄ alkyl group substituents; and
a group R²⁰-Q- where R²⁰ is phenyl optionally substituted with one to three substituents selected from C₁₋₄ alkyl, trifluoromethyl, fluoro and chloro; and Q is a linker group selected from OCH₂, CH₂O, NH, CH₂NH, NCH₂, CH₂, NHCO and CONH.

In one preferred sub-group of compounds, R^{1a} is selected from heteroaryl groups having 5 or 6 ring members (e.g. oxazole, thiazole, pyridyl, pyrimidinyl) and containing up to 3 heteroatoms selected from N, O and S, the heteroaryl groups being optionally substituted with one to three substituents selected from C₁₋₄ alkyl, trifluoromethyl, fluoro and chloro. A substituted thiazole group, for example, 2-methyl-4-trifluoromethyl-2-thiazolyl, represents one preferred embodiment.

In another preferred sub-group of compounds, R^{1a} is selected from 5-membered monocyclic heteroaryl groups containing a single oxygen heteroatom ring member and optionally a nitrogen heteroatom ring member, and being substituted by one to three substituents R^{10c} provided that when the heteroaryl group contains a nitrogen ring member and is substituted by a methyl group, at least one substituent other than methyl is present. One such group is isoxazole substituted by a C₂₋₄ alkyl group such as a propyl or butyl group, e.g. isobutyl.

In another preferred sub-group of compounds, R^{1a} is selected from three and five membered cycloalkyl groups substituted by one to three substituents R^{10c}. Substituted cyclopropyl groups are particularly preferred, for example cyclopropyl group substituted by phenyl or cyano, e.g. 1-cyanocyclopropyl and 1-phenylcyclopropyl.

In a further sub-group of compounds, R^{1a} is selected from a group Ph′CR¹⁷R¹⁸— where Ph′ is a phenyl group substituted by one to three substituents R_{10c}; R¹⁷ and R¹⁸ are the same or different and each is selected from hydrogen and methyl; or R¹⁷ and R¹⁸ together with the carbon atom to which they are attached form a cyclopropyl group; or one of R¹⁷ and R¹⁸ is hydrogen and the other is selected from amino, methylamino, C₁₋₄ acylamino, and C₁₋₄ alkoxycarbonylamino.

Another group of novel compounds of the invention can be represented by the formula (V):

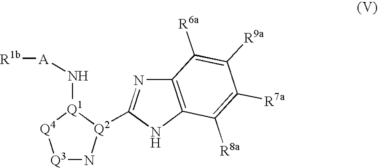

wherein
A is NH(C═O) or C═O;
R^{1b} is a substituted phenyl group having from 1 to 4 substituents whereby:
(i) when R^{1b} bears a single substituent it is selected from halogen, hydroxyl, C₁₋₄ hydrocarbyloxy optionally substituted by one or more substituents selected from hydroxyl and halogen; C₁₋₄ hydrocarbyl substituted by one or more substituents selected from hydroxyl and halogen; heteroaryl groups having 5 ring members; and 5- and 6-membered non-aromatic heterocyclic groups, wherein the heteroaryl and heterocyclic groups contain up to 3 heteroatoms selected from N, O and S;
(ii) when R^{1b} bears 2, 3 or 4 substituents, each is selected from halogen, hydroxyl, C₁₋₄ hydrocarbyloxy optionally substituted by one or more substituents selected from hydroxyl and halogen; C₁₋₄ hydrocarbyl optionally substituted by one or more substituents selected from hydroxyl and halogen; heteroaryl groups having 5 ring members; amino; and 5- and 6-membered non-aromatic heterocyclic groups; or two adjacent substituents together with the carbon atoms to which they are attached form a 5-membered heteroaryl ring or a 5- or 6-membered non-aromatic heterocyclic ring; wherein the said heteroaryl and heterocyclic groups contain up to 3 heteroatoms selected from N, O and S; and
   R^{6a}, R^{7a}, R^{8a} and R^{9a} are as hereinbefore defined.

The group R^{1a}-A-NH or R^{1b}-A-NH linked to Q¹ can take the form of an amide R^{1a/1b}—C(═O)NH, urea R^{1a/1b}—NHC(═O) or carbamate R^{1a/1b}—OC(═O). Amides and ureas are preferred. In one embodiment, the compound is an amide. In another embodiment, the compound is a urea.

In formula (V), the substituted phenyl group R^{1b} is substituted by a single substituent as hereinbefore defined, or by more than one substituent. Thus, there may be 1 or 2 or 3 or 4 substituents, more preferably 1, 2 or 3 substituents. In one embodiment, there may be two or three substituents and these may be located at the 2-, 3-, 4-, 5- or 6-positions around the ring.

By way of example, a phenyl group R^{1b} may be 2,6-disubstituted, 2,3-disubstituted, 2,4-disubstituted 2,5-disubstituted, 2,3,6-trisubstituted or 2,4,6-trisubstituted. In one group of preferred compounds, the phenyl group R^{1b} is 2,6-disubstituted, 2,3-disubstituted or 2,4,6-trisubstituted. More particularly, a phenyl group R^{1b} may be disubstituted at positions 2- and 6- with substituents selected from fluorine, chlorine and R^{a}-R^{b}, where R^{a} is O and R^{b} is C₁₋₄ alkyl, with fluorine being a particular substituent. Alternatively, two adjacent substituents (preferably in the 2- and 3-positions), together with the phenyl ring to which they are attached, may form a 2, 3-dihydro-benzo[1,4]dioxine group, or an indolyl group or a 2,3-dihydrobenzofuranyl group.

In another group of preferred compounds, the phenyl group R^{1b} is 2,4-disubstituted or 2,5-disubstituted. The 2-substituent may be, for example, a halogen (e.g. F or Cl) or a methoxy group. In one particular group of compounds, the 2-substituent is methoxy. The 5-substituent, when present, can be selected from, for example, halogen (e.g. Cl or F), C₁₋₄ alkyl (e.g. tert-butyl or isopropyl), methoxy, trifluoromethoxy, trifluoromethyl, or a group HetN—SO₂— where “HetN” is a nitrogen-containing saturated monocyclic heterocycle such as piperazino, N—C₁₋₄ alkylpiperazino, morpholino, piperidino or pyrrolidino. One preferred 5-subsitutent is Cl, and a preferred 2,5-combination is 2-methoxy-5-chlorophenyl.

In a further group of compounds, the phenyl group R^{1b} has a single substituent at the 4-position of the phenyl ring. The substituent can be, for example, a halogen atom (preferably fluorine or chlorine, most preferably fluorine) or a trifluoromethyl group.

In another group of compounds, the phenyl group R^{1b} is 2,4-disubstituted.

When two adjacent substituents together with the phenyl ring to which they are attached form an indolyl group or a 2,3-dihydrobenzofuranyl group, it is preferred that the said groups are the 4-indolyl and 7-(2,3-dihydrobenzofuranyl) groups respectively.

Where R^{1b} is mono-substituted, and the substituent is located at the 4-position of the phenyl ring, it is preferably other than a difluoromethoxy group or a 2-chloroethyl group (although the 4-(2-chloroethyl)-phenyl group may serve as an intermediate to other compounds of the formula (V)).

In one embodiment, where R^{1b} is disubstituted, the substituted phenyl group may be other than a dimethoxyphenyl group, and may be other than a 2-fluoro-5-methoxyphenyl group.

In another embodiment, the sub-group R^{1b} may include the 2-fluoro-5-methoxyphenyl group. Such compounds have good activity against Aurora kinase.

Where two adjacent substituents combine to form a ring so that R^{1b} is an indole group, the indole group is preferably other than an indol-7-yl group.

One preferred sub-group of compounds of the invention is the group wherein R^{1b} is selected from the groups A1 to A8, A10, A12 and A14 to A24 set out in Table 1 above.

Particularly preferred groups R^{1′} include 2,6-difluorophenyl, 2-fluoro-6-methoxyphenyl, 2-chloro-6-fluorophenyl, 2,6-dichlorophenyl, 2,4,6-trifluorophenyl and 2,3-dihydro-benzo[1,4]dioxine.

One currently preferred group R^{1′} is 2,6-difluorophenyl.

The moieties R^{6a}, R^{7a}, R^{8a} and R^{9a} are typically selected from hydrogen, halogen, hydroxy, trifluoromethyl, cyano, nitro, carboxy, amino, monocyclic carbocyclic and heterocyclic groups having from 3 to 12 (preferably 3 to 7, and more typically 5 or 6) ring members, a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, a carbocyclic or heterocyclic group with 3-7 ring members and a C₁₋₈ hydrocarbyl group optionally substituted by one or more substituents selected from hydroxy, C₁₋₄ acyloxy, oxo, halogen, cyano, nitro, carboxy, amino, mono- or di-C₁₋₄ hydrocarbylamino, a carbocyclic or heterocyclic group with 3-7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; and R^{c}, X¹ and X²; or an adjacent pair of substituents selected from R^{6a}, R^{7a}, R^{8a} and R^{9a} together with the carbon atoms to which they are attached may form a non-aromatic five or six membered ring containing up to three heteroatoms selected from O, N and S.

In one embodiment, R^{6a} to R^{9a} are each hydrogen or are selected from halogen, cyano, hydroxy, trifluoromethyl, nitro, a group R^{a}-R^{b} wherein R^{a} is a bond, O, CO or C(X²)X¹ and R^{b} is selected from hydrogen, heterocyclic groups having from 3 to 12 ring members (preferably 4 to 7 ring members), and a C₁₋₈ hydrocarbyl group (preferably a C₁₋₄ hydrocarbyl group), optionally substituted by one or more substituents selected from hydroxy, C₁₋₄ acyloxy, mono- or di-C₁₋₄ hydrocarbylamino, heterocyclic groups having from 3 to 12 ring members, more preferably 4 to 7 ring members; where R^{c} is selected from hydrogen and C₁₋₄ hydrocarbyl, X¹ is O or NR^{c} and X² is ═O.

In another embodiment, R^{6a}, R^{7a}, R^{8a} and R^{9a} are selected from hydrogen, fluorine, chlorine, bromine, nitro, trifluoromethyl, carboxy, a group R^{a}-R^{b} wherein R^{a} is a bond, O, CO, C(X²)X¹, and R^{b} is selected from hydrogen, heterocyclic groups having 3-7 (preferably 5 or 6) ring members (e.g. pyrrolidine, N-methyl piperazine or morpholine) and a C₁₋₄ hydrocarbyl group optionally substituted by one or more substituents selected from hydroxy, carboxy, C¹⁻⁴ acyloxy, amino, mono- or di-C₁₋₄ hydrocarbylamino, heterocyclic groups with 3-7 (preferably 5 or 6) ring members (e.g. pyrrolidine, N-methyl piperazine or morpholine); or an adjacent pair of substituents selected from R^{6a}, R^{7a}, R^{8a} and R^{9a} together with the carbon atoms to which they are attached may form a non-aromatic five or six membered ring containing one or two oxygen atoms as ring members.

In a more preferred embodiment, R^{6a}, R^{7a}, R^{8a} and R^{9a} are selected from hydrogen, fluorine, chlorine, trifluoromethyl, a group R^{a}-R^{b} wherein R^{a} is a bond, O, CO, C(X²)X¹, and R^{b} is selected from hydrogen, saturated heterocyclic groups having 5-6 ring members and a C₁₋₂ hydrocarbyl group (e.g. alkyl) optionally substituted by one or more substituents selected from hydroxy, carboxy, C₁₋₂ acyloxy, amino, mono- or di-C₁₋₄ hydrocarbylamino (e.g. mono- or dialkylamino), heterocyclic groups with 5-6 ring members; or an adjacent pair of substituents selected from R^{6a}, R^{7a}, R^{8a} and R^{9a} may form a methylenedioxy or ethylenedioxy group each optionally substituted by one or more fluorine atoms.

In another embodiment, particular substituent groups R^{6a} to R^{9a} include halogen, nitro, carboxy, a group R^{a}-R^{b} wherein R^{a} is a bond, O, CO, C(X²)X¹, and R^{b} is selected from hydrogen, heterocyclic group having 3-7 ring members (preferably 5 or 6 ring members) and a C₁₋₄ hydrocarbyl group (e.g. alkyl or cycloalkyl) optionally substituted by one or more substituents selected from hydroxy, carboxy, amino, mono- or di-C₁₋₄ hydrocarbylamino (e.g. mono- or di-alkylamino), heterocyclic group with 3-7 ring members (preferably 5 or 6 ring members).

Whereas each of R^{6a} to R₉ₐ can be hydrogen or a substituent other than hydrogen as hereinbefore defined, it is preferred that at least one, more preferably at least two, of R^{6a} to R^{9a} are hydrogen.

In one particular embodiment, one of R^{6a} to R^{9a} is a substituent other than hydrogen and the others each are hydrogen. For example, R^{6a} can be a substituent group other than hydrogen and R^{7a} to R^{9a} can each be hydrogen, or R^{9a} can be a substituent other than hydrogen and R^{6a}, R^{7a} and R^{8a} can each be hydrogen.

In another particular embodiment, two of R^{6a} to R^{9a} are substituents other than hydrogen and the other two are both hydrogen. For example, R^{6a} and R^{9a} can both be substituents other than hydrogen when R^{7a} and R^{8a} are both hydrogen; or R^{6a} and R^{7a} can both be substituents other than hydrogen when R^{9a} and R^{8a} are both hydrogen; or R^{9a} and R^{7a} can both be substituents other than hydrogen when R^{6a} and R^{8a} are both hydrogen.

R^{6a} is preferably selected from:

hydrogen;

halogen (preferably fluorine or chlorine);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R^{9a} is preferably selected from: hydrogen;

halogen (preferably fluorine or chlorine);

C₁₋₄ alkoxy (for example methoxy);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R^{7a} is preferably selected from:

hydrogen;

halogen (preferably fluorine or chlorine);

C₁₋₄ alkoxy (for example methoxy);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R^{8a} is preferably selected from hydrogen, fluorine and methyl, most preferably hydrogen.

Alternatively, R^{6a} and R^{9a}, or R^{9a} and R^{7a}, together with the carbon atoms to which they are attached may form a cyclic group selected from:

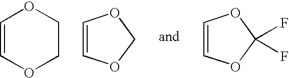

In the foregoing definitions, when R¹¹ and R¹² together with the nitrogen atom in the group NR¹¹R¹² form a five or six membered heterocyclic ring, the heteroatom ring members are preferably selected from O and N. The heterocyclic ring is typically non-aromatic and examples of such rings include morpholine, piperazine, N—C₁₋₄-alkylpiperazine, piperidine and pyrrolidine. Particular examples of N—C₁₋₄-alkylpiperazine groups include N-methylpiperazine and N-isopropylpiperazine.

Preferred groups R^{6a} to R^{9a} include those in which the benzimidazole group

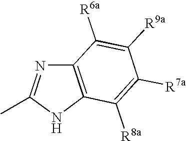

is as shown in Table 2 above.

Of the benzimidazole groups set out in Table 2 above, particular groups include groups B1, B3, B5-B8, B11-B20, B23-B30 and B32-B47.

Particularly preferred groups are groups B1, B3, B5-B8, B11-B20, B24, B25, B27-B30 and B32-B47.

One preferred group of compounds of the formula (V) can be represented by the formula (Va):

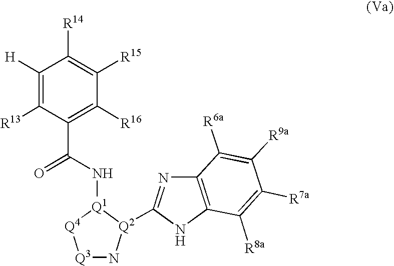

wherein R^{6a} to R^{9a} are as hereinbefore defined; and
(i) R¹³ is methoxy and R¹⁴ to R¹⁶ each are hydrogen; or
(ii) R¹⁴ is oxazolyl, imidazolyl or thiazolyl, preferably oxazolyl, and R¹³, R¹⁵ and R¹⁶ each are hydrogen; or
(iii) R¹³ is selected from fluorine, chlorine and methyl, R¹⁶ is selected from fluorine, chlorine, methyl and methoxy, and R¹⁴ and R¹⁵ each are hydrogen; or
(iv) R¹³ and R¹⁶ each are selected from fluorine, chlorine and methyl; R¹⁴ is selected from fluorine, chlorine, methyl and methoxy; and R¹⁵ is hydrogen; or
(v) R¹³ and R¹⁴ each are hydrogen; R¹⁵ is selected from fluorine, chlorine, methyl and methoxy (more preferably methyl and methoxy), and R¹⁶ is selected from fluorine, chlorine and methyl (more preferably fluorine), or R¹⁵ and R¹⁶ together with the carbon atoms of the phenyl ring form a group selected from:

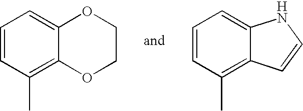

Particularly preferred substituents for the phenyl ring are the groups of substituents (i), (iii), (iv) and (v).

Within formula (Va), one particular sub-group of compounds is the group of compounds wherein:
(i) R¹³ is methoxy and R¹⁴ to R¹⁶ each are hydrogen; or
(iii) R¹³ is selected from fluorine, chlorine and methyl, R¹⁶ is selected from fluorine, chlorine, methyl and methoxy, and R¹⁴ and R¹⁵ each are hydrogen; or
(vi) R¹³ and R¹⁶ each are selected from fluorine, chlorine and methyl; R¹⁴ is selected from fluorine, chlorine and methoxy; and R¹⁵ is hydrogen; or
(vii) R¹³ and R¹⁴ each are hydrogen, R¹⁵ is methoxy and R¹⁶ is fluorine, or R¹⁵ and R¹⁶ together with the carbon atoms of the phenyl ring form a group selected from:

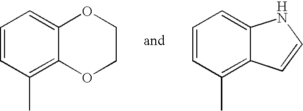

A particularly preferred sub-group of compounds within formula (Va) is the group of compounds wherein:
(iii) R¹³ is selected from fluorine, chlorine and methyl, R¹⁶ is selected from fluorine, chlorine, methyl and methoxy, and R¹⁴ and R¹⁵ each are hydrogen; or
(vi) R¹³, R¹⁴ and R¹⁶ each are fluorine and R¹⁵ is hydrogen; or
(vii) R¹³ and R¹⁴ each are hydrogen and R¹⁵ and R¹⁶ together with the carbon atoms of the phenyl ring form a group:

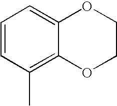

Compounds of the formulae (V) and (Va) are particularly preferred as inhibitors of CDK.

In a further embodiment, the invention provides a compound of the formula (VI):

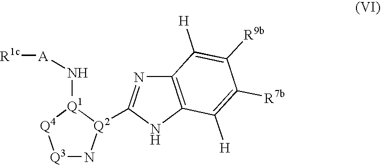

wherein:

when A is NH(C═O) or C═O;

R^{1c} is selected from:
(a) a mono-substituted phenyl group wherein the substituent is selected from o-amino, o-methoxy; o-chloro; p-chloro; o-difluoromethoxy; o-trifluoromethoxy; o-tert-butyloxy; m-methylsulphonyl and p-fluoro;
(b) a 2,4- or 2,6-disubstituted phenyl group wherein one substituent is selected from o-methoxy, o-ethoxy, o-fluoro, p-morpholino and the other substituent is selected from o-fluoro, o-chloro, p-chloro, and p-amino;
(c) a 2,5-disubstituted phenyl group wherein one substituent is selected from o-fluoro and o-methoxy and the other substituent is selected from m-methoxy, m-isopropyl; m-fluoro, m-trifluoromethoxy, m-trifluoromethyl, m-methylsulphanyl, m-pyrrolidinosulphonyl, m-(4-methylpiperazin-1-yl)sulphonyl, m-morpholinosulphonyl, m-methyl, m-chloro and m-aminosulphonyl;
(d) a 2,4,6-tri-substituted phenyl group where the substituents are the same or different and are each selected from o-methoxy, o-fluoro, p-fluoro, p-methoxy provided that no more than one methoxy substituent is present;
(e) a 2,4,5-tri-substituted phenyl group where the substituents are the same or different and are each selected from o-methoxy, m-chloro and p-amino;
(f) unsubstituted benzyl; 2,6-difluorobenzyl; α,α-dimethylbenzyl; 1-phenylcycloprop-1-yl; and α-tert-butoxycarbonylaminobenzyl;
(g) an unsubstituted 2-furyl group or a 2-furyl group bearing a single substituent selected from 4-(morpholin-4-ylmethyl), piperidinylmethyl; and optionally a further substituent selected from methyl;
(h) an unsubstituted pyrazolo[1,5-a]pyridin-3-yl group;
(i) isoxazolyl substituted by one or two C₁₋₄ alkyl groups;
(j) 4,5,6,7-tetrahydro-benzo[d]isoxazol-3-yl;
(k) 3-tert-butyl-phenyl-1H-pyrazol-5-yl;
(l) quioxalinyl;
(m) benzo[c]isoxazol-3-yl;
(n) 2-methyl-4-trifluoromethyl-thiazol-5-yl;
(o) 3-phenylamino-2-pyridyl;
(p) 1-toluenesulphonylpyrrol-3-yl;
(q) 2,4-dimethoxy-3-pyridyl; and 6-chloro-2-methoxy-4-methyl-3-pyridyl;
(r) imidazo[2,1-b]thiazol-6-yl;
(s) 5-chloro-2-methylsulphanyl-pyrimidin-4-yl;
(t) 3-methoxy-naphth-2-yl;
(u) 2,3-dihydro-benzo[1,4]dioxin-5-yl;
(v) 2,3-dihydro-benzofuranyl group optionally substituted in the five membered ring by one or two methyl groups;
(w) 2-methyl-benzoxazol-7-yl;
(x) 4-aminocyclohex-1-yl;
(y) 1,2,3,4-tetrahydro-quinolin-6-yl;
(z) 2-methyl-4,5,6,7-tetrahydro-benzofuran3-yl;
(aa) 2-pyrimidinyl-1piperidin-4-yl; and 1-(5-trifluoromethyl-2-pyridyl)-piperidin-4-yl and 1-methylsulphonylpiperidin-4-yl;
(ab) 1-cyanocyclopropyl;
(ac) N-benzylmorpholin-2-yl;

and when A is NH(C═O), R^{1′} is additionally selected from:
(ad) unsubstituted phenyl;

R^{9b} is selected from hydrogen; chlorine; methoxy; methylsulphonyl; 4-methyl-piperazin-1-ylcarbonyl; morpholinocarbonyl; morpholinomethyl; pyrrolidinylcarbonyl; N-methyl-piperidinyloxy; pyrrolidinylethoxy; morpholinopropylaminomethyl; 4-cyclopentyl-piperazin-1-ylmethyl; 4-ethylsulphonyl-piperazin-1-ylmethyl; morpholinosulphonyl; 4-(4-methylcyclohexyl)-piperazin-1-ylmethyl; and

R^{7b} is selected from hydrogen; methyl; methoxy and ethoxy.

Compounds of the formula (VI) have good activity against Aurora kinases.

Preferred compounds of the formula (VI) are those that have a mean IC₅₀ against Aurora kinase A of less than 0.03 μM, and more preferably 0.01 μM or less when determined by the methods described herein.

One particular sub-group of compounds of the formula (VI) is the group of compounds in which R^{9b} is selected from morpholinomethyl and methoxy, and R^{7b} is methoxy when R^{9b} is methoxy, or R^{7b} is hydrogen when R^{9b} is morpholinomethyl.

A further group of novel compounds of the invention can be represented by the formula (VII):

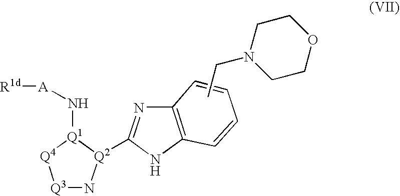

wherein R^{1d} is a group R¹, R^{1a}, R^{1b} or R^{1c} as hereinbefore defined.

In one particular sub-group of compounds within formula (VII), A is NH(C═O) and R^{1d} is unsubstituted C₃₋₆ cycloalkyl or a group R^{1c} as defined herein.

The C₃₋₆ cycloalkyl group can be cyclopropyl, cyclobutyl, cyclopentyl or cyclohexyl but preferably is cyclopropyl.

Preferred compounds within this sub-group are the compounds wherein R^{1d} is unsubstituted cyclopropyl or 2,6-difluorophenyl.

Compounds of the formula (VII) show good CDK inhibitory activity and are also particularly active against Aurora kinases.

A particularly preferred sub-group of compounds within formula (VII) is represented by formula (VIIa):

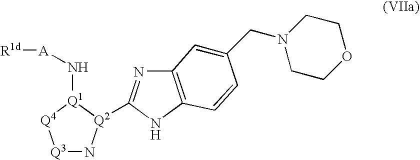

where R^{1d} is as hereinbefore defined.

Another sub-group of novel compounds of the invention is represented by formula (VIII):

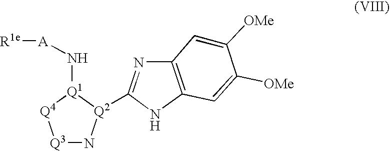

where R^{1c} is a group R¹a or a group R¹b as hereinbefore defined.

A further group of novel compounds of the invention is represented by general formula (IX):

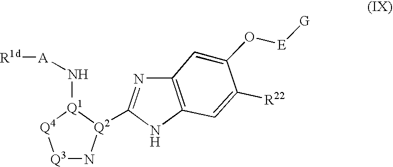

wherein R^{1d} is as defined herein, E is a bond, CH₂ or CH₂CH₂, R²² is selected from hydrogen, halogen (e.g. fluorine or chlorine), and C₁₋₂ alkoxy (e.g methoxy), and G is a 4-7 membered saturated heterocyclic ring containing up to 3 heteroatom ring members selected from N, O and S, the heterocyclic ring being optionally substituted by 1 to 4 (preferably up to 2, e.g. 0 or 1) groups R¹⁰ (or a sub group thereof as defined herein).

Within formula (IX), one particular group of compounds is represented by formula (IXa):

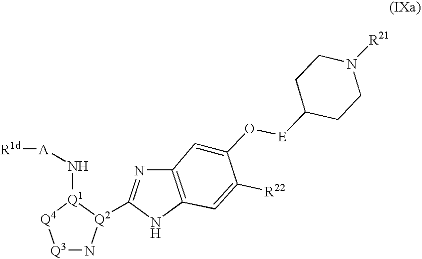

Wherein R^{1d}, E and R²² are as defined herein and R²¹ is selected from hydrogen, C₁₋₄ alkyl (e.g. methyl), C₁₋₄ acyl, and C₁₋₄ alkoxycarbonyl. A preferred combination is the combination in which E is CH₂, R²¹ is methyl and R²² is methoxy.

For the avoidance of doubt, it is to be understood that each general and specific preference, embodiment and example of the groups R¹ may be combined with each general and specific preference, embodiment and example of the groups R² and/or R³ and/or R⁴ and/or R⁵ and/or R⁶ and/or R⁷ and/or R⁸ and/or R⁹ and/or R¹⁰ and any sub-groups thereof and that all such combinations are embraced by this application.

For example, any one of the groups R¹ (e.g. as in R¹-A where A is C═O) shown in Table 1 may be combined with any one of the benzimidazole groups shown in Table 2.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular and specific compounds of the invention are as illustrated in the examples below, and/or include:

N-[4-(1H-benzoimidazol-2-yl)-thiazol-5-yl]-2,6-difluoro-benzamide;

2,6-difluoro-N-[4-(6-morpholin-4-ylmethyl-1H-benzoimidazol-2-yl)-thiazol-5-yl]benzamide;

2,6-difluoro-N-[3-(5-morpholin-4-ylmethyl-1H-indol-2-yl)-isothiazol-4-yl]-benzamide;

2,3-dihydro-benzofuran-5-carboxylic acid [4-(6-morpholin-4-ylmethyl-1H-benzoimidazol-2-yl)-thiazol-5-yl]-amide;

2-chloro-4-morpholin-4-yl-N-[4-(6-morpholin-4-ylmethyl-1H-benzoimidazol-2-yl)-thiazol-5-yl]-benzamide;

pyrrolidine-2-carboxylic acid [4-(5,6-dimethoxy-1H-benzoimidazol-2-yl)-thiazol-5-yl]-amide;

1-methyl-piperidine-4-carboxylic acid [4-(5,6-dimethoxy-1H-benzoimidazol-2-yl)-thiazol-5-yl]-amide; and

1-cyclopropyl-3-[3-(5-morpholin-4-ylmethyl-1 H-benzoimidazol-2-yl)-thiazol-5-yl]-urea;

1-(2,6-difluorophenyl)-3-[3-(5-morpholin-4-ylmethyl-1H-benzoimidazol-2-yl)-thiazol-5-yl]-urea;

1-cyclopropyl-3-[3-(5-morpholin-4-ylmethyl-1H-benzoimidazol-2-yl)-isothiazol-4-yl]-urea;

1-(2,6-difluorophenyl)-3-[3-(5-morpholin-4-ylmethyl-1H-benzoimidazol-2-yl)-isothiazol-4-yl]-urea;

and salts, tautomers, N-oxides and solvates thereof.
Salts, Solvates, Tautomers. Isomers, N-Oxides, Esters, Prodrugs and Isotopes

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formula (II) and sub-groups thereof unless the context indicates otherwise.

Salt forms may be selected and prepared according to methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. For example, acid addition salts may be prepared by dissolving the free base in an organic solvent in which a given salt form is insoluble or poorly soluble and then adding the required acid in an appropriate solvent so that the salt precipitates out of solution.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

The acid addition salts may also be selected from aspartic (e.g. D-aspartic), carbonic, dodecanoate, isobutyric, laurylsulphonic, mucic, naphthalenesulphonic (e.g. naphthalene-2-sulphonic), toluenesulphonic (e.g. p-toluenesulphonic), and xinafoic acids.

One particular group of acid addition salts includes salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

Salts such as acid addition salts have a number of advantages over the corresponding free base. For example, the salts will enjoy one or more of the following advantages over the free base in that they will:
be more soluble and hence will be better for i.v. administration (e.g. by infusion) and will have improved pharmacokinetics;
have better stability (e.g. improved shelf life);
have better thermal stability;
be less basic and therefore better for i.v. administration;
have advantages for production;
have improved metabolic properties; and
exhibit less clinical variation between patients.

Preferred salts for use in the preparation of liquid (e.g. aqueous) compositions of the compounds of formula (I) and sub-groups and examples thereof as described herein are salts having a solubility in a given liquid carrier (e.g. water) of greater than 25 mg/ml of the liquid carrier (e.g. water), more typically greater than 50 mg/ml and preferably greater than 100 mg/ml.

In another embodiment preferred salts for use in the preparation of liquid (e.g. aqueous) compositions the compounds of formula (I) and sub-groups and examples thereof as described herein are salts having a solubility in a given liquid carrier (e.g. water) greater than 1 mg/ml, typically greater than 5 mg/ml of the liquid carrier (e.g. water), more typically greater than 15 mg/ml, more typically greater than 20 mg/ml and preferably greater than 25 mg/ml.

In another embodiment of the invention, there is provided a pharmaceutical composition comprising an aqueous solution containing a compound of the formula (I) and sub-groups and examples thereof as described herein in the form of a salt in a concentration of greater than 1 mg/ml, typically greater than 5 mg/ml of the liquid carrier (e.g. water), more typically greater than 15 mg/ml, more typically greater than 20 mg/ml and preferably greater than 25 mg/ml.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth metal cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium salts are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, in compounds of the formula (I) the benzimidazole group may take either of the following two tautomeric forms A and B. For simplicity, the general formula (I) illustrates form A but the formula is to be taken as embracing both tautomeric forms.

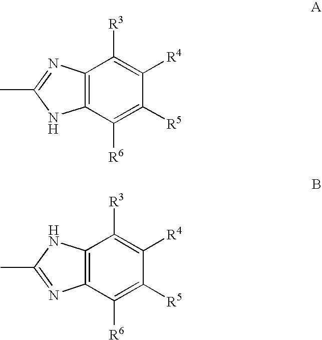

Other examples of tautomeric forms include, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

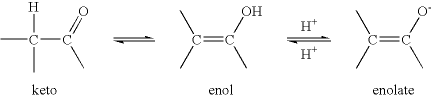

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures or two or more optical isomers, unless the context requires otherwise.

For example, the group A can include one or more chiral centres. Thus, when E and R¹ are both attached to the same carbon atom on the linker group A, the said carbon atom is typically chiral and hence the compound of the formula (I) will exist as a pair of enantiomers (or more than one pair of enantiomers where more than one chiral centre is present in the compound).

The optical isomers may be characterised and identified by their optical activity (i.e. as + and − isomers, or d and I isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluloyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇ alkyl

(e.g., -Me, -Et, -nPr, -iPr, -nBu, -sBu, -iBu, -tBu);

C₁₋₇ aminoalkyl

(e.g., aminoethyl; 2-(N,N-diethylamino)ethyl; 2-(4-morpholino)ethyl); and acyloxy-C₁₋₇ alkyl

(e.g., acyloxymethyl;

acyloxyethyl;

pivaloyloxymethyl;

acetoxymethyl;

1-acetoxyethyl;

1-(1-methoxy-1-methyl)ethyl-carbonxyloxyethyl;

1-(benzoyloxy)ethyl; isopropoxy-carbonyloxymethyl;

1-isopropoxy-carbonyloxyethyl; cyclohexyl-carbonyloxymethyl;

1-cyclohexyl-carbonyloxyethyl;

cyclohexyloxy-carbonyloxymethyl;

1-cyclohexyloxy-carbonyloxyethyl;

(4-tetrahydropyranyloxy) carbonyloxymethyl;

1-(4-tetrahydropyranyloxy)carbonyloxyethyl;

(4-tetrahydropyranyl)carbonyloxymethyl; and

1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Biological Activity

The compounds of the invention have cyclin dependent kinase inhibiting or modulating activity and glycogen synthase kinase-3 (GSK3) inhibiting or modulating activity, and/or Aurora kinase inhibiting or modulating activity, and which it is envisaged will be useful in preventing or treating disease states or conditions mediated by the kinases.

Thus, for example, it is envisaged that the compounds of the invention will be useful in alleviating or reducing the incidence of cancer.

More particularly, the compounds of the formula (J) and sub-groups thereof are inhibitors of cyclin dependent kinases. For example, compounds of the invention have activity against CDK1, CDK2, CDK3, CDK4, CDK5, CDK6 and CDK7 kinases, and in particular cyclin dependent kinases selected from CDK1, CDK2, CDK3, CDK4, CDK5 and CDK6.

Preferred compounds are compounds that inhibit one or more CDK kinases selected from CDK1, CDK2, CDK4 and CDK5, for example CDK1 and/or CDK2.

In addition, CDK4, CDK8 and/or CDK9 may be of interest.

Compounds of the invention also have activity against glycogen synthase kinase-3 (GSK-3).

Compounds of the invention also have activity against Aurora kinases. Preferred compounds of the invention are those having IC₅₀ values of less than 0.1 μM.

Many of the compounds of the invention exhibit selectivity for the Aurora A kinase compared to CDK1 and CDK2 and such compounds represent one preferred embodiment of the ivention. For example, many compounds of the invention have IC₅₀ values against Aurora A that are between a tenth and a hundredth of the IC₅₀ against CDK1 and CDK2.

As a consequence of their activity in modulating or inhibiting CDK and Aurora kinases and glycogen synthase kinase, they are expected to be useful in providing a means of arresting, or recovering control of, the cell cycle in abnormally dividing cells. It is therefore anticipated that the compounds will prove useful in treating or preventing proliferative disorders such as cancers. It is also envisaged that the compounds of the invention will be useful in treating conditions such as viral infections, type II or non-insulin dependent diabetes mellitus, autoimmune diseases, head trauma, stroke, epilepsy, neurodegenerative diseases such as Alzheimer's, motor neurone disease, progressive supranuclear palsy, corticobasal degeneration and Pick's disease, for example.

One sub-group of disease states and conditions where it is envisaged that the compounds of the invention will be useful consists of viral infections, autoimmune diseases and neurodegenerative diseases.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve tumours may be particularly sensitive to CDK inhibitors. RB−ve tumours may also be sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; or Kaposi's sarcoma.

The cancers may be cancers which are sensitive to inhibition of any one or more cyclin dependent kinases selected from CDK1, CDK2, CDK3, CDK4, CDK5 and CDK6, for example, one or more CDK kinases selected from CDK1, CDK2, CDK4 and CDK5, e.g. CDK1 and/or CDK2.

Whether or not a particular cancer is one which is sensitive to inhibition by a cyclin dependent kinase or an aurora kinase may be determined by means of a cell growth assay as set out in the examples below or by a method as set out in the section headed “Methods of Diagnosis”.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-senstive rhinosinusitis, cystic fibrosis, multiple sclerosis, kidney diseases and cancer pain.

It has also been discovered that some cyclin-dependent kinase inhibitors can be used in combination with other anticancer agents. For example, the cyclin-dependent kinase inhibitor flavopiridol has been used with other anticancer agents in combination therapy.

Thus, in the pharmaceutical compositions, uses or methods of this invention for treating a disease or condition comprising abnormal cell growth, the disease or condition comprising abnormal cell growth in one embodiment is a cancer.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes breast cancer, ovarian cancer, colon cancer, prostate cancer, oesophageal cancer, squamous cancer and non-small cell lung carcinomas.

In the case of compounds having activity against Aurora kinase, particular examples of cancers where it is envisaged that the Aurora kinase inhibiting compounds of the invnention will be useful include:

human breast cancers (e.g. primary breast tumours, node-negative breast cancer, invasive duct adenocarcinomas of the breast, non-endometrioid breast cancers);

ovarian cancers (e.g. primary ovarian tumours);

pancreatic cancers;

human bladder cancers;

colorectal cancers (e.g. primary colorectal cancers);

gastric tumours;

renal cancers;

cervical cancers:

neuroblastomas;

melanomas;

lymphomas;

prostate cancers;

leukemia;

non-endometrioid endometrial carcinomas;

gliomas; and

non-Hodgkin's lymphoma.

Cancers which may be particularly amenable to Aurora inhibitors include breast, bladder, colorectal, pancreatic, ovarian, non-Hodgkin's lymphoma, gliomas and nonendometrioid endometrial carcinomas.

A particular sub-set of cancers which may be particularly amenable to Aurora inhibitors consist of breast, ovarian, colon, liver, gastric and prostate cancers.

Another subset of cancers that Aurora inhibitors may be particularly amenable to treat consists of hematological cancers, in particular leukemia. Therefore, in a further embodiment the compounds of formula (I) are used to treat hematological cancers, in particular leukemia. Particular leukemias are selected from Acute Myelogenous Leukemia (AML), chronic myelogenous leukaemia (CML), B-cell lymphoma (Mantle cell), and Acute Lymphoblastic Leukemia (ALL). In one embodiment the leukemias are selected from relapsed or refractory acute myelogenous leukemia, myelodysplastic syndrome, acute lymphocytic leukemia and chronic myelogenous leukemia.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes hematopoietic tumours of lymphoid lineage, for example leukemia, chronic lymphocytic leukaemia, mantle cell lymphoma and B-cell lymphoma (such as diffuse large B cell lymphoma).

One particular cancer is chronic lymphocytic leukaemia.

Another particular cancer is mantle cell lymphoma.

Another particular cancer is diffuse large B cell lymphoma.

It is further envisaged that the compounds of the invention, and in particular those compounds having aurora kinase inhibitory activity, will be particularly useful in the treatment or prevention of cancers of a type associated with or characterised by the presence of elevated levels of aurora kinases, for example the cancers referred to in this context in the introductory section of this application.

The activity of the compounds of the invention as inhibitors of cyclin dependent kinases, Aurora kinases and glycogen synthase kinase-3 can be measured using the assays set forth in the examples below and the level of activity exhibited by a given compound can be defined in terms of the IC₅₀ value. Preferred compounds of the present invention are compounds having an IC₅₀ value of less than 1 μM, more preferably less than 0.1 μM.

### Methods for the Preparation of Compounds of the Formula (I)

Compounds of the formula (I) can be prepared in accordance with synthetic methods well known to the skilled person.

Unless stated otherwise R¹, R², R³ and R⁴ are as herein defined.

Compounds of the formula (I) wherein R¹-A- forms an acyl group can be prepared as illustrated in Scheme 1 below.

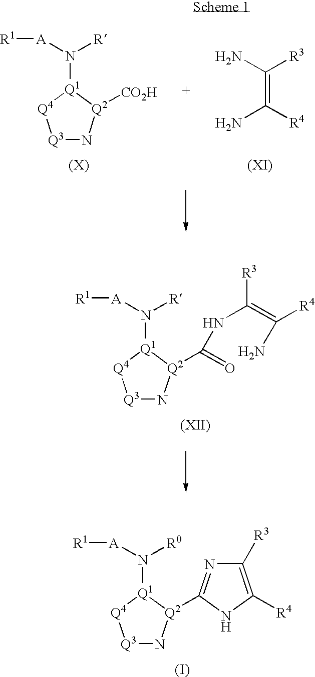

As shown in Scheme 1, a carboxylic acid of the formula (X) is reacted with a diamine of the formula (XI) in a ring forming reaction to give the bicyclic imidazole (e.g. benzimidazole) group. In compound (X), the group R′ can be a group R⁰ or an N-protecting group such as para-methoxybenzyl.

The ring forming reaction typically takes place in two stages. The first stage involves forming an amide bond between one of the amino groups of the diamine and the carboxylic acid to give a mono-amide intermediate (XII). This reaction can be carried out using standard amide formation conditions. Thus, for example, the coupling reaction between the carboxylic acid and the diamine (XI) can be carried out in the presence of a reagent of the type commonly used in the formation of peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (EDC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) (L. A. Carpino, J. Amer. Chem. Soc., 1993, 115, 4397) and phosphonium-based coupling agents such as 1-benzo-triazolyloxytris(pyrrolidino)phosphonium hexafluorophosphate (PyBOP) (Castro et al, Tetrahedron Letters, 1990, 31, 205). Carbodiimide-based coupling agents are advantageously used in combination with 1-hydroxyazabenzotriazole (HOAt) or 1-hydroxybenzotriazole (HOBt) (Konig et al, Chem. Ber., 103, 708, 2024-2034). Preferred coupling reagents include EDC and DCC in combination with HOAt or HOBt.

The coupling reaction is typically carried out in a non-aqueous, non-protic solvent such as acetonitrile, dioxane, dimethylsulphoxide, dichloromethane, dimethylformamide or N-methylpyrrolidone, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

Once the amide bond has been formed between the carboxylic acid (X) and the diamine (XI), the intermediate amide (XII) can either be isolated and characterised or carried directly through to the next stage in which cyclisation to form the imidazole ring is brought about by heating in acetic acid, for example to a temperature up to about 125° C. Once the cyclisation has taken place, any protecting groups R′ can be removed to give a compound of the formula (I).

Diamines of the formula (XI) can be obtained commercially or can be prepared from appropriately substituted phenyl precursor compounds using standard chemistry and well known functional group interconversions, see for example, Fiesers' Reagentsfor Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995.

Carboxylic acids of the formula (X) can either be obtained commercially or can be prepared by methods known to those skilled in the art.

Carboxylic acids of the formula (X) wherein Q³ is S can be formed by the sequence of reactions shown in Scheme 2.

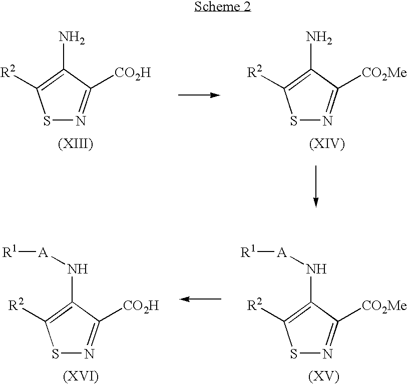

As shown in Scheme 2, the 4-amino-isothiazol-3-yl carboxylic acid (XII) is esterified to give the ester (XIV). Esterification can be carried out under standard conditions, for example by reacting the acid with methanol in the presence of thionyl chloride. The amino group of the ester (XIV) can then be converted to a compound of the formula (XV) by reaction with an appropriate reagent. For example, a carboxylic acid of the formula R¹—CO₂H or R¹—(CH²)ₘ—CO₂H can be reacted with the ester (XIV) under amide forming conditions of the type described above to give compounds wherein R¹-A-N(R⁰)—forms an amide group.

Alternatively, the amino-group of the ester (XIV) can be converted into a urea by reaction with an isocyanate of the formula R¹—N═C═O or R¹—(CH₂)ₘ—N═C═O under standard urea forming conditions. Ureas may alternatively be formed by reacting the ester (XIV) with an amine R¹—NH₂ or R¹—(CH₂)ₘ—NH₂ in the presence of a “carbonyl donating” reagent such as carbonyl dimidazole (CDI) or triphosgene. The ester (XV) is then hydrolysed to give the carboxylic acid (XVI) using an alkali metal hydroxide such as sodiuym hydroxide.

Carboxylic acids of the formula (X) wherein Q⁴ is S can be formed by the sequence of reactions shown in Scheme 3.

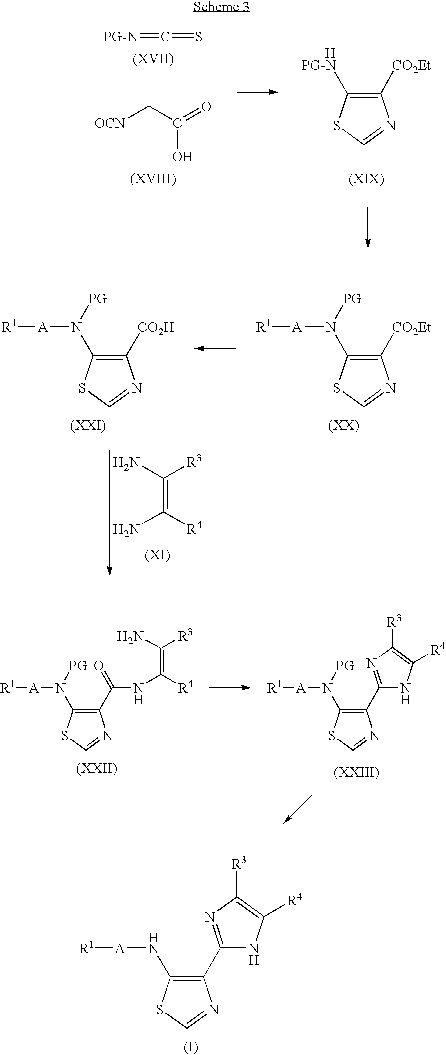

In Scheme 3, ethyl isocyanoacetate (XVIII) is reacted with a substituted isothiocyanate (XVII) in which PG is a protecting group such as p-methoxybenzyl to form thiazole ester (XIX). The reaction is typically carried out in a polar solvent such as THF in the presence of a strong base such as potassium tert-butoxide, for example at room temperature.

The thiazole ester is then converted into the ester compound (XX) by reaction with a carboxylic acid or active derivative thereof under amide forming conditions, or by reaction with appropriately substituted isocyanate or amine under urea forming conditions as described above in conection with Scheme 2.

The ester compound (XX) is then hydroysed using an alkali metal hydroxide such as sodium hydroxide to give the carboxylic acid (XXI).

The carboxylic acid (XXI) is then reacted with a diamine (XI) to give intermediate amide (XXII) which is then cyclised to the compound of formula (I) by the methods decribed above in connection with Scheme 1.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999). A hydroxy group may be protected, for example, as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(—O)CH₃).

### Methods of Purification

The compounds may be isolated and purified by a number of methods well known to those skilled in the art and examples of such methods include chromatographic techniques such as column chromatography (e.g. flash chromatography) and HPLC. Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described in the experimental section below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

### Recrystallisation

Methods of recrystallisation of compounds of formula (I) and salt thereof can be carried out by methods well known to the skilled person—see for example P. Heinrich Stahl (Editor), Camille G. Wermuth (Editor), ISBN: 3-90639-026-8, Handbook of Pharmaceutical Salts: Properties, Selection, and Use, Chapter 8, Publisher Wiley-VCH. Products obtained from an organic reaction are seldom pure when isolated directly from the reaction mixture. If the compound (or a salt thereof) is solid, it may be purified and/or crystallized by recrystallization from a suitable solvent. A good recrystallization solvent should dissolve a moderate quantity of the substance to be purified at elevated temperatures but only a small quantity of the substance at lower temperature. It should dissolve impurities readily at low temperatures or not at all. Finally, the solvent should be readily removed from the purified product. This usually means that it has a relatively low boiling point and a person skilled in the art will know recrystallizing solvents for a particular substance, or if that information is not available, test several solvents. To get a good yield of purified material, the minimum amount of hot solvent to dissolve all the impure material is used. In practice, 3-5% more solvent than necessary is used so the solution is not saturated. If the impure compound contains an impurity which is insoluble in the solvent it may then be removed by filtration and then allowing the solution to crystallize. In addition, if the impure compound contains traces of coloured material that are not native to the compound, it may be removed by adding a small amount of decolorizing charcoal to the hot solution, filtering it and then allowing it to crystallize. Usually crystallization spontaneously occurs upon cooling the solution. If it is not, crystallization may be induced by cooling the solution below room temperature or by adding a single crystal of pure material (a seed crystal). Recrystallisation can also be carried out and/or the yield optimized by the use of an anti-solvent. In this case, the compound is dissolved in a suitable solvent at elevated temperature, filtered and then an additional solvent in which the required compound has low solubility is added to aid crystallization. The crystals are then typically isolated using vacuum filtration, washed and then dried, for example, in an oven or via desiccation.

Other examples of methods for crystallization include crystallization from a vapour, which includes an evaporation step for example in a sealed tube or an air stream, and crystallization from melt (Crystallization Technology Handbook 2nd Edition, edited by A. Mersmann, 2001).

In particular the compound of formula (I) may subjected to recrystallisation (e.g. using 2-propanol or ethanol as the solvent) to increase the purity and to give a crystalline form.

The crystals obtained may be analysed by an X-ray diffraction method such as X-ray powder diffraction (XRPD) or X-ray crystal diffraction to determine their crystal structure.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF).

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides a compound of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Examples of these are described in R. G. Strickly, Solubilizing Excipients in oral and injectable formulations, Pharmaceutical Research, Vol 21(2) 2004, p 201-230. In addition, they may contain co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

A drug molecule that is ionizable can be solubilized to the desired concentration by pH adjustment if the drug's pKa is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at −5-20 mg/ml, an isotonicifier, a pH 5-8 buffer, and optionally cholesterol.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of Formula (I) or acid addition salt thereof. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediamietetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

Pharmaceutical compositions of the present invention for parenteral injection can also comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If a compound is not stable in aqueous media or has low solubility in aqueous media, it can be formulated as a concentrate in organic solvents. The concentrate can then be diluted to a lower concentration in an aqueous system, and can be sufficiently stable for the short period of time during dosing. Therefore in another aspect, there is provided a pharmaceutical composition comprising a non aqueous solution composed entirely of one or more organic solvents, which can be dosed as is or more commonly diluted with a suitable IV excipient (saline, dextrose; buffered or not buffered) before administration (Solubilizing excipients in oral and injectable formulations, Pharmaceutical Research, 21(2), 2004, p 201-230). Examples of solvents and surfactants are propylene glycol, PEG300, PEG400, ethanol, dimethylacetamide (DMA), N-methyl-2-pyrrolidone (NMP, Pharmasolve), Glycerin, Cremophor EL, Cremophor RH 60 and polysorbate. Particular non aqueous solutions are composed of 70-80% propylene glycol, and 20-30% ethanol. One particular non aqueous solution is composed of 70% propylene glycol, and 30% ethanol. Another is 80% propylene glycol and 20% ethanol.Normally these solvents are used in combination and usually diluted at least 2-fold before IV bolus or IV infusion. The typical amounts for bolus IV formulations are ˜50% for Glycerin, propylene glycol, PEG300, PEG400, and ˜20% for ethanol. The typical amounts for IV infusion formulations are ˜15% for Glycerin, 3% for DMA, and ˜10% for propylene glycol, PEG300, PEG400 and ethanol.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The compounds of the invention will generally be presented in unit dosage form and, as such, will typically contain sufficient compound to provide a desired level of biological activity. For example, a formulation intended for oral administration may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, for example, 50 milligrams to 500 milligrams or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

### Methods of Treatment

It is envisaged that the compounds of the invention as defined herein will be useful in the prophylaxis or treatment of a range of disease states or conditions mediated by cyclin dependent kinases, glycogen synthase kinase-3 and Aurora kinases. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, such as 1 micrograms to 10 milligrams) per kilogram of bodyweight although higher or lower doses may be administered where required. Ultimately, the quantity of compound administered and the type of composition used will be commensurate with the nature of the disease or physiological condition being treated and will be at the discretion of the physician.

The compounds of the invention as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or therapies that may be administered or used together (whether concurrently or at different time intervals) with the compounds of the invention include but are not limited to topoisomerase inhibitors, alkylating agents, antimetabolites, DNA binders, microtubule inhibitors (tubulin targeting agents), particular examples being cisplatin, cyclophosphamide, doxorubicin, irinotecan, fludarabine, 5FU, taxanes, mitomycin C and radiotherapy.

Other examples of therapeutic agents that may be administered together (whether concurrently or at different time intervals) with the compounds of the formulae (I), (II), (III) and sub-groups as defined herein include monoclonal antibodies and signal transduction inhibitors.

For the case of CDK or Aurora inhibitors combined with other therapies, the two or more treatments may be given in individually varying dose schedules and via different routes.

Where the compound of the formula (I) is administered in combination therapy with one, two, three, four or more other therapeutic agents (preferably one or two, more preferably one), the compounds can be administered simultaneously (either in the same or different pharmaceutical formulation) or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against Aurora and/or cyclin dependent kinases.

For example, a biological sample taken from a patient may be analysed to determine whether a condition or disease, such as cancer, that the patient is or may be suffering from is one which is characterised by a genetic abnormality or abnormal protein expression which leads to over-activation of CDKs or to sensitisation of a pathway to normal CDK activity. Examples of such abnormalities that result in activation or sensitisation of the CDK2 signal include up-regulation of cyclin E, (Harwell R M, Mull B B, Porter D C, Keyomarsi K.; J Biol Chem. Mar. 26 2004;279(13):12695-705) or loss of p21 or p27, or presence of CDC4 variants (Rajagopalan H, Jallepalli P V, Rago C, Velculescu V E, Kinzler K W, Vogelstein B, Lengauer C.; Nature. Mar. 4, 2004;428(6978):77-81). Tumours with mutants of CDC4 or up-regulation, in particular over-expression, of cyclin E or loss of p21 or p27 may be particularly sensitive to CDK inhibitors. Alternatively or in addition, a biological sample taken from a patient may be analysed to determine whether a condition or disease, such as cancer, that the patient is or may be suffering from is one which is characterised by upregulation of Aurora kinase and thus may be particularly to Aurora inhibitors. The term up-regulation includes elevated expression or over-expression, including gene amplification (i.e. multiple gene copies) and increased expression by a transcriptional effect, and hyperactivity and activation, including activation by mutations.

Thus, the patient may be subjected to a diagnostic test to detect a marker characteristic of over-expression, up-regulation or activation of Aurora kinase or the patient may be subjected to a diagnostic test to detect a marker characteristic of up-regulation of cyclin E, or loss of p21 or p27, or presence of CDC4 variants. The term diagnosis includes screening. By marker we include genetic markers including, for example, the measurement of DNA composition to identify mutations of Aurora or CDC4. The term marker also includes markers which are characteristic of up regulation of Aurora or cyclin E, including enzyme activity, enzyme levels, enzyme state (e.g. phosphorylated or not) and mRNA levels of the aforementioned proteins. Tumours with upregulation of cyclin E, or loss of p21 or p27 may be particularly sensitive to CDK inhibitors. Tumours may preferentially be screened for upregulation of cyclin E, or loss of p21 or p27 prior to treatment. Thus, the patient may be subjected to a diagnostic test to detect a marker characteristic of up-regulation of cyclin E, or loss of p21 or p27.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

It has been found, see Ewart-Toland et al., (Nat Genet. August 2003;34(4):403-12), that individuals forming part of the sub-population possessing the Ile31 variant of the STK gene (the gene for Aurora kinase A) may have an increased susceptibility to certain forms of cancer. It is envisaged therefore that such individuals suffering from cancer will benefit from the administration of compounds having Aurora kinase inhibiting activity. A patient suffering from, or suspected of suffering from, a cancer may therefore be screened to determine whether he or she forms part of the Ile31 variant sub-population. In addition, it has been found, Rajagopalan et al (Nature. Mar 4, 2004;428(6978):77-81), that there were mutations present in CDC4 (also known as Fbw7 or Archipelago) in human colorectal cancers and endometrial cancers (Spruck et al, Cancer Res. Aug 15, 2002;62(16):4535-9). Identification of individual carrying a mutation in CDC4 may mean that the patient would be particularly suitable for treatment with a CDK inhibitor. Tumours may preferentially be screened for presence of a CDC4 variant prior to treatment. The screening process will typically involve direct sequencing, oligonucleotide microarray analysis, or a mutant specific antibody.

Tumours with activating mutants of Aurora or up-regulation of Aurora including any of the isoforms thereof, may be particularly sensitive to Aurora inhibitors. Tumours may preferentially be screened for up-regulation of Aurora or for Aurora possessing the Ile31 variant prior to treatment (Ewart-Toland et al., Nat Genet. August 2003;34(4):403-12). Ewart-Toland et al identified a common genetic variant in STK15 (resulting in the amino acid substitution F311) that is preferentially amplified and associated with the degree of aneuploidy in human colon tumors. These results are consistent with an important role for the Ile31 variant of STK15 in human cancer susceptibility. In particular, this polymorphism in Aurora A has been suggested to be a genetic modifier fir developing breast carcinoma (Sun et al, Carcinogenesis, 2004, 25(11), 2225-2230).

The aurora A gene maps to the chromosome 20q13 region that is frequently amplified in many cancers e.g. breast, bladder, colon, ovarian, pancreatic. Patients with a tumour that has this gene amplification might be particularly sensitive to treatments targeting aurora kinase inhibition

Methods of identification and analysis of mutations and up-regulation of protein e.g. Aurora isoforms and chromosome 20q13 amplification are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of cyclin E, or loss of p21 or p27, or detection of CDC4 variants, Aurora up-regulation and mutants of Aurora could be applicable in the present case.

Therefore, all of these techniques could also be used to identify tumours particularly suitable for treatment with the compounds of the invention.

Tumours with mutants of CDC4 or up-regulation, in particular over-expression, of cyclin E or loss of p21 or p27 may be particularly sensitive to CDK inhibitors. Tumours may preferentially be screened for up-regulation, in particular over-expression, of cyclin E (Harwell R M, Mull B B, Porter D C, Keyomarsi K.; J Biol Chem. Mar. 26, 2004;279(13):12695-705) or loss of p21 or p27 or for CDC4 variants prior to treatment (Rajagopalan H, Jallepalli P V, Rago C, Velculescu V E, Kinzler K W, Vogelstein B, Lengauer C.; Nature. Mar. 24, 2004;428(6978):77-81).

Patients with mantle cell lymphoma (MCL) could be selected for treatment with a compound of the invention using diagnostic tests outlined herein. MCL is a distinct clinicopathologic entity of non-Hodgkin's lymphoma, characterized by proliferation of small to medium-sized lymphocytes with co-expression of CD5 and CD20, an aggressive and incurable clinical course, and frequent t(11;14)(q13;q32) translocation. Over-expression of cyclin D1 mRNA, found in mantle cell lymphoma (MCL), is a critical diagnostic marker. Yatabe et al (Blood. Apr. 1, 2000;95(7):2253-61) proposed that cyclin D1-positivity should be included as one of the standard criteria for MCL, and that innovative therapies for this incurable disease should be explored on the basis of the new criteria. Jones et al (J Mol Diagn. May 2004;6(2):84-9) developed a real-time, quantitative, reverse transcription PCR assay for cyclin D1 (CCND1) expression to aid in the diagnosis of mantle cell lymphoma (MCL). Howe et al (Clin Chem. January 2004;50(1):80-7) used real-time quantitative RT-PCR to evaluate cyclin D1 mRNA expression and found that quantitative RT-PCR for cyclin D1 mRNA normalized to CD19 mRNA can be used in the diagnosis of MCL in blood, marrow, and tissue. Alternatively, patients with breast cancer could be selected for treatment with a CDK inhibitor using diagnostic tests outline above. Tumour cells commonly overexpress cyclin E and it has been shown that cyclin E is over-expressed in breast cancer (Harwell et al, Cancer Res, 2000, 60, 481-489). Therefore breast cancer may in particular be treated with a CDK inhibitor as provided herein.

### Antifungal Use

In a further aspect, the invention provides the use of the compounds of the invention as antifungal agents.

The compounds of the invention may be used in animal medicine (for example in the treatment of mammals such as humans), or in the treatment of plants (e.g. in agriculture and horticulture), or as general antifungal agents, for example as preservatives and disinfectants.

In one embodiment, the invention provides a compound of the invention for use in the prophylaxis or treatment of a fungal infection in a mammal such as a human.

Also provided is the use of a compound of the invention for the manufacture of a medicament for use in the prophylaxis or treatment of a fungal infection in a mammal such as a human.

For example, compounds of the invention may be administered to human patients suffering from, or at risk of infection by, topical fungal infections caused by among other organisms, species of Candida, Trichophyton, Microsporum or Epidermophyton, or in mucosal infections caused by Candida albicans (e.g. thrush and vaginal candidiasis). The compounds of the invention can also be administered for the treatment or prophylaxis of systemic fungal infections caused by, for example, Candida albicans, Cryptococcus neoformans, Aspergillus flavus, Aspergillus fumigatus, Coccidiodies, Paracoccidioides, Histoplasma or Blastomyces.

In another aspect, the invention provides an antifungal composition for agricultural (including horticultural) use, comprising a compound of the formulae (I), (II), (III) and sub-groups thereof as defined herein together with an agriculturally acceptable diluent or carrier.

The invention further provides a method of treating an animal (including a mammal such as a human), plant or seed having a fungal infection, which comprises treating said animal, plant or seed, or the locus of said plant or seed, with an effective amount of a compound of the invention.

The invention also provides a method of treating a fungal infection in a plant or seed which comprises treating the plant or seed with an antifungally effective amount of a fungicidal composition containing a compound of the invention.

Differential screening assays may be used to select for those compounds of the present invention with specificity for non-human CDK enzymes. Compounds which act specifically on the CDK enzymes of eukaryotic pathogens can be used as anti-fungal or anti-parasitic agents. Inhibitors of the Candida CDK kinase, CKSI, can be used in the treatment of candidiasis. Antifungal agents can be used against infections of the type hereinbefore defined, or opportunistic infections that commonly occur in debilitated and immunosuppressed patients such as patients with leukemias and lymphomas, people who are receiving immunosuppressive therapy, and patients with predisposing conditions such as diabetes mellitus or AIDS, as well as for non-immunosuppressed patients.

Assays described in the art can be used to screen for agents which may be useful for inhibiting at least one fungus implicated in mycosis such as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocardiosis, para-actinomycosis, penicilliosis, monoliasis, or sporotrichosis. The differential screening assays can be used to identify anti-fungal agents which may have therapeutic value in the treatment of aspergillosis by making use of the CDK genes cloned from yeast such as Aspergillus fumigatus, Aspergillus flavus, Aspergillus niger, Aspergillus nidulans, or Aspergillus terreus, or where the mycotic infection is mucon-nycosis, the CDK assay can be derived from yeast such as Rhizopus arrhizus, Rhizopus oryzae, Absidia corymbifera, Absidia ramosa, or Mucorpusillus. Sources of other CDK enzymes include the pathogen Pneumocystis carinii.

By way of example, in vitro evaluation of the antifungal activity of the compounds can be performed by determining the minimum inhibitory concentration (M.I.C.) which is the concentration of the test compounds, in a suitable medium, at which growth of the particular microorganism fails to occur. In practice, a series of agar plates, each having the test compound incorporated at a particular concentration is inoculated with a standard culture of, for example, Candida albicans and each plate is then incubated for an appropriate period at 37° C. The plates are then examined for the presence or absence of growth of the fungus and the appropriate M.I.C. value is noted. Alternatively, a turbidity assay in liquid cultures can be performed and a protocol outlining an example of this assay can be found in the examples below.

The in vivo evaluation of the compounds can be carried out at a series of dose levels by intraperitoneal or intravenous injection or by oral administration, to mice that have been inoculated with a fungus, e.g., a strain of Candida albicans or Aspergillus flavus. The activity of the compounds can be assessed by monitoring the growth of the fungal infection in groups of treated and untreated mice (by histology or by retrieving fungi from the infection). The activity may be measured in terms of the dose level at which the compound provides 50% protection against the lethal effect of the infection (PD₅₀).

For human antifungal use, the compounds of the invention can be administered alone or in admixture with a pharmaceutical carrier selected in accordance with the intended route of administration and standard pharmaceutical practice. Thus, for example, they may be administered orally, parenterally, intravenously, intramuscularly or subcutaneously by means of the formulations described above in the section headed “Pharmaceutical Formulations”.

For oral and parenteral administration to human patients, the daily dosage level of the antifungal compounds of the invention can be from 0.01 to 10 mg/kg (in divided doses), depending on inter alia the potency of the compounds when administered by either the oral or parenteral route. Tablets or capsules of the compounds may contain, for example, from 5 mg to 0.5 g of active compound for administration singly or two or more at a time as appropriate. The physician in any event will determine the actual dosage (effective amount) which will be most suitable for an individual patient and it will vary with the age, weight and response of the particular patient.

Alternatively, the antifungal compounds can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin; or they can be incorporated, at a concentration between 1 and 10%, into an ointment consisting of a white wax or white soft paraffin base together with such stabilizers and preservatives as may be required.

In addition to the therapeutic uses described above, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms. In similar fashion, side by side comparison of inhibition of a mammalian CDK and an insect CDK, such as the Drosophilia CDK5 gene (Hellmich et al. (1994) FEBS Lett 356:317-21), will permit selection amongst the compounds herein of inhibitors which discriminate between the human/mammalian and insect enzymes. Accordingly, the present invention expressly contemplates the use and formulation of the compounds of the invention in insecticides, such as for use in management of insects like the fruit fly.

In yet another embodiment, certain of the subject CDK inhibitors can be selected on the basis of inhibitory specificity for plant CDK's relative to the mammalian enzyme. For example, a plant CDK can be disposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

For agricultural and horticultural purposes the compounds of the invention may be used in the form of a composition formulated as appropriate to the particular use and intended purpose. Thus the compounds may be applied in the form of dusting powders, or granules, seed dressings, aqueous solutions, dispersions or emulsions, dips, sprays, aerosols or smokes. Compositions may also be supplied in the form of dispersible powders, granules or grains, or concentrates for dilution prior to use. Such compositions may contain such conventional carriers, diluents or adjuvants as are known and acceptable in agriculture and horticulture and they can be manufactured in accordance with conventional procedures. The compositions may also incorporate other active ingredients, for example, compounds having herbicidal or insecticidal activity or a further fungicide. The compounds and compositions can be applied in a number of ways, for example they can be applied directly to the plant foliage, stems, branches, seeds or roots or to the soil or other growing medium, and they may be used not only to eradicate disease, but also prophylactically to protect the plants or seeds from attack. By way of example, the compositions may contain from 0.01 to 1 wt.% of the active ingredient. For field use, likely application rates of the active ingredient may be from 50 to 5000 g/hectare.

The invention also contemplates the use of the compounds of the invention in the control of wood decaying fungi and in the treatment of soil where plants grow, paddy fields for seedlings, or water for perfusion. Also contemplated by the invention is the use of the compounds of the invention to protect stored grain and other non-plant loci from fungal infestation.

### EXAMPLES

The invention will now be illustrated, but not limited, by reference to the specific embodiments described in the following examples. In the examples, the starting materials are commercially available or are preparable by methods well known to those skilled in the art unless otherwise indicated.

In the examples, the following abbreviations may be used.
DCM dichloromethane
DMF dimethylformamide
DMSO dimethyl sulphoxide
EDC 1-ethyl-3-(3′-dimethylaminopropyl)-carbodiimide
Et₃N triethylamine
EtOAc ethyl acetate
Et₂O diethyl ether
HOAt 1-hydroxyazabenzotriazole
HOBt 1-hydroxybenzotriazole
MeCN acetonitrile
MeOH methanol
PMB para-methoxybenzyl
SiO₂ silica
TBTU N,N,N′,N′-tetramethyl-O-(benzotriazol-1-yl)uronium tetrafluoroborate
THF tetrahydrofuran

### Analytical LC-MS System and Method Description

In the examples, the compounds prepared were characterised by liquid chromatography and mass spectroscopy using the systems and operating conditions set out below. Where atoms with different isotopes are present, and a single mass quoted, the mass quoted for the compound is the monoisotopic mass (i.e. ³⁵Cl; ⁷⁹Br etc.). Several systems were used, as described below, and these were equipped with, and were set up to run under, closely similar operating conditions. The operating conditions used are also described below.

### Waters Platform LC-MS System:

| | |
|---|---|
| HPLC System: | Waters 2795 |
| Mass Spec Detector: | Micromass Platform LC |
| PDA Detector: | Waters 2996 PDA |

### Analytical Acidic Conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ MAX-RP 80A, 2.0 × 50 mm |

### Analytical Basic Conditions:

| | |
|---|---|
| Eluent A: | H₂O (10 mM NH₄HCO₃ buffer adjusted to pH = 9.2 with |
| | NH₄OH) |
| Eluent B: | CH₃CN |
| Gradient: | 05-95% eluent B over 3.5 minutes |
| Flow: | 0.8 ml/min |
| Column: | Phenomenex Luna C18(2) 5 μm 2.0 × 50 mm |

### Analytical Polar Conditions:

| | |
|---|---|
| Eluent A: | H₂O (0.1% Formic Acid) |
| Eluent B: | CH₃CN (0.1% Formic Acid) |
| Gradient: | 00-50% eluent B over 3 minutes |
| Flow: | 0.8 ml/min |
| Column: | Phenomenex Synergi 4μ MAX-RP 80A, 2.0 × 50 mm |

### Analytical Lipophilic Conditions:

| | |
|---|---|
| Eluent A: | H₂O (0.1% Formic Acid) |
| Eluent B: | CH₃CN (0.1% Formic Acid) |
| Gradient: | 55-95% eluent B over 3.5 minutes |
| Flow: | 0.8 ml/min |
| Column: | Phenomenex Synergi 4μ MAX-RP 80A, 2.0 × 50 mm |

### Analytical Long Acidic Conditions:

| | |
|---|---|
| Eluent A: | H₂O (0.1% Formic Acid) |
| Eluent B: | CH₃CN (0.1% Formic Acid) |
| Gradient: | 05-95% eluent B over 15 minutes |
| Flow: | 0.4 ml/min |
| Column: | Phenomenex Synergi 4μ MAX-RP 80A, 2.0 × 150 mm |

### Analytical Long Basic Conditions:

| | |
|---|---|
| Eluent A: | H₂O (10 mM NH₄HCO₃ buffer adjusted to pH = 9.2 |
| | with NH₄OH) |
| Eluent B: | CH₃CN |
| Gradient: | 05-95% eluent B over 15 minutes |
| Flow: | 0.8 ml/min |
| Column: | Phenomenex Luna C18(2) 5 μm 2.0 × 50 mm |

### Platform MS Conditions:

| | |
|---|---|
| Capillary voltage: | 3.6 kV (3.40 kV on ES negative) |
| Cone voltage: | 25 V |
| Source Temperature: | 120° C. |
| Scan Range: | 100-800 amu |
| Ionisation Mode: | ElectroSpray Positive or |
| | ElectroSpray Negative or |
| | ElectroSpray Positive & Negative |

### Waters Fractionlynx LC-MS System:

| | |
|---|---|
| HPLC System: | 2767 autosampler-2525 binary gradient pump |
| Mass Spec Detector: | Waters ZQ |
| PDA Detector: | Waters 2996 PDA |

### Analytical Acidic Conditions:

| | |
|---|---|
| Eluent A: | H₂O (0.1% Formic Acid) |
| Eluent B: | CH₃CN (0.1% Formic Acid) |
| Gradient: | 5-95% eluent B over 4 minutes |
| Flow: | 2.0 ml/min |
| Column: | Phenomenex Synergi 4μ MAX-RP 80A, 4.6 × 50 mm |

### Analytical Polar Conditions:

| | |
|---|---|
| Eluent A: | H₂O (0.1% Formic Acid) |
| Eluent B: | CH₃CN (0.1% Formic Acid) |
| Gradient: | 00-50% eluent B over 4 minutes |
| Flow: | 2.0 ml/min |
| Column: | Phenomenex Synergi 4μ MAX-RP 80A, 4.6 × 50 mm |

### Analytical Lipophilic Conditions:

| | |
|---|---|
| Eluent A: | H₂O (0.1% Formic Acid) |
| Eluent B: | CH₃CN (0.1% Formic Acid) |
| Gradient: | 55-95% eluent B over 4 minutes |
| Flow: | 2.0 ml/min |
| Column: | Phenomenex Synergi 4μ MAX-RP 80A, 4.6 × 50 mm |

### Fractionlynx MS Conditions:

| | |
|---|---|
| Capillary voltage: | 3.5 kV (3.2 kV on ES negative) |
| Cone voltage: | 25 V (30 V on ES negative) |
| Source Temperature: | 120° C. |
| Scan Range: | 100-800 amu |
| Ionisation Mode: | ElectroSpray Positive or |
| | ElectroSpray Negative or |
| | ElectroSpray Positive & Negative |

### Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

Preparative LC-MS Systems:

### Waters Fractionlynx System:

Hardware:
2767 Dual Loop Autosampler/Fraction Collector
2525 preparative pump
CFO (column fluidic organiser) for column selection
RMA (Waters reagent manager) as make up pump
Waters ZQ Mass Spectrometer
Waters 2996 Photo Diode Array detector
Waters ZQ Mass Spectrometer

Software:
Masslynx 4.0

Waters MS Running Conditions:

| | |
|---|---|
| Capillary voltage: | 3.5 kV (3.2 kV on ES Negative) |
| Cone voltage: | 25 V |
| Source Temperature: | 120° C. |
| Multiplier: | 500 V |
| Scan Range: | 125-800 amu |
| Ionisation Mode: | ElectroSpray Positive or |
| | ElectroSpray Negative |

### Agilent 1100 LC-MS Preparative System:

Hardware:
Autosampler: 1100 series “prepALS”
Pump: 1100 series “PrepPump” for preparative flow gradient and 1100 series
“QuatPump” for pumping modifier in prep flow
UV detector: 1100 series “MWD” Multi Wavelength Detector
MS detector: 1100 series “LC-MSD VL”
Fraction Collector: 2× “Prep-FC”
Make Up pump: “Waters RMA”
Agilent Active Splitter

Software:
Chemstation: Chem32

Agilent MS Running Conditions:

| | |
|---|---|
| Capillary voltage: | 4000 V (3500 V on ES Negative) |
| Fragmentor/Gain: | 150/1 |
| Drying gas flow: | 13.0 L/min |
| Gas Temperature: | 350° C. |
| Nebuliser Pressure: | 50 psig |
| Scan Range: | 125-800 amu |
| Ionisation Mode: | ElectroSpray Positive or |
| | ElectroSpray Negative |

### Chromatographic Conditions

Columns:

1. Low pH chromatography:

Phenomenex Synergy MAX-RP, 10 μ, 100×21.2 mm (alternatively used Thermo Hypersil-Keystone HyPurity Aquastar, 5μ, 100×21.2 mm for more polar compounds)

2. High pH chromatography:

Phenomenex Luna C18 (2), 10μ, 100×21.2 mm (alternatively used Phenomenex Gemini, 5μ, 100×21.2 mm)

Eluents:

1. Low pH chromatography:

Solvent A: H₂O+0.1% Formic Acid, pH˜1.5

Solvent B: CH₃CN+0.1% Formic Acid

2. High pH chromatography:

Solvent A: H₂O+10 mM NH₄HCO₃+NH4OH, pH=9.2

Solvent B: CH₃CN

3. Make up solvent:

MeOH+0.2% Formic Acid (for both chromatography type)

Methods:

According to the analytical trace the most appropriate preparative chromatography type was chosen. A typical routine was to run an analytical LC-MS using the type of chromatography (low or high pH) most suited for compound structure. Once the analytical trace showed good chromatography a suitable preparative method of the same type was chosen. Typical running condition for both low and high pH chromatography methods were:

Flow rate: 24 ml/min

Gradient: Generally all gradients had an initial 0.4 min step with 95% A+5% B. Then according to analytical trace a 3.6 min gradient was chosen in order to achieve good separation (e.g. from 5% to 50% B for early retaining compounds; from 35% to 80% B for middle retaining compounds and so on)

Wash: 1.2 minute wash step was performed at the end of the gradient

Re-equilibration: 2.1 minutes re-equilibration step was ran to prepare the system for the next run

Make Up flow rate: 1 ml/min

Solvent:

All compounds were usually dissolved in. 100% MeOH or 100% DMSO

From the information provided someone skilled in the art could purify the compounds described herein by preparative LC-MS.

### EXAMPLE 1

### Synthesis of N-[4-(1H-benzoimidazol-2-yl)-thiazol-5-yl]-2,6-difluoro-benzamide

### 1A. Synthesis of 5-(4-methoxy-benzylamino)-thiazole-4-carboxylic acid ethyl ester

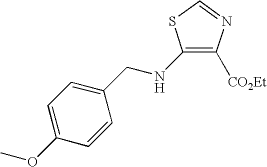

To a vigorously stirred solution of potassium tert-butoxide (5.45 g, 48.59 mmoles) in THF (140 ml) was added dropwise ethyl isocyanoacetate (4.8 ml, 44.17 mmoles). The suspension was stirred at ambient temperature for 10 minutes. To the suspension was added dropwise 4-methoxybenzyl isothiocyanate (6.9 ml, 44.17 mmoles). The suspension was stirred at ambient temperature for a further 2 hours. Acetic acid (10 ml) was added to the suspension and then the solvent was removed in vacuo. The residue was partitioned between EtOAc and water. The organic portion was dried (MgSO₄), filtered and evaporated in vacuo. The residue was purified [Biotage SP4, 2×40M, flow rate 40 ml/min, gradient 1:4 EtOAc/petrol to 7:3 EtOAc/petrol] to give 5-(4-methoxy-benzylamino)-thiazole-4-carboxylic acid ethyl ester as a brown oil (7.6 g, 59%). (LC/MS: Rₜ2.90, [M+H]⁺ 292.99).

### 1B. Synthesis of 5-[2,6-difluoro-benzoyl)-(4-methoxy-benzyl)-amino]-thiazole-4-carboxylic acid ethyl ester

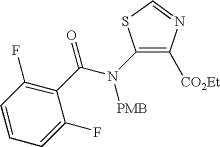

To a stirred solution of 5-(4-methoxy-benzylamino)-thiazole-4-carboxylic acid ethyl ester (1.0 g, 3.42 mmoles) in DMF (10 ml) was added portionwise sodium hydride (301 mg, 7.53 mmoles). The solution was stirred at ambient temperature for 10 minutes. To the reaction mixture was added 2,6-difluorobenzoyl chloride (0.858 ml, 6.84 mmoles), and the mixture was then stirred at ambient temperature for 1 hour before partitioning between ether and water. The organic portion was dried (MgSO₄), filtered and evaporated in vacuo. The residue was purified [Biotage SP4, 40S, flow rate 40 ml/min, gradient 1:4 EtOAc/petrol to 7:3 EtOAc/petrol) to give 5-[2,6-difluoro-benzoyl)-(4-methoxy-benzyl)-amino]-thiazole-4-carboxylic acid ethyl ester as a white solid (1.1 g, 74%). (LC/MS: Rₜ 3.16, [M+H]⁺ 432.98).

### 1C. Synthesis of 5-[(2,6-difluoro-benzoyl)-(4-methoxy-benzyl)-amino]-thiazole-4-carboxylic acid

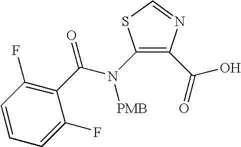

A solution of 5-[2,6-difluoro-benzoyl)-(4-methoxy-benzyl)-amino]-thiazole-4-carboxylic acid ethyl ester (1.1 g, 2.55 mmoles) in a mixture of ethanol (20 ml) and 2N sodium hydroxide solution (20 ml) was stirred at ambient temperature for 24 hours. Ethanol was evaporated in vacuo. The residue was partitioned between EtOAc and 2N hydrochloric acid. The organic portion was dried (MgSO₄), filtered and evaporated in vacuo to give 5-[(2,6-difluoro-benzoyl)-(4-methoxy-benzyl)-amino]-thiazole-4-carboxylic acid as a pale yellow solid (0.95 g, 92%). (LC/MS: Rt 2.68, [M+H]⁺ 404.92).

### 1D. Synthesis of 5-[(2,6-difluoro-benzoyl)-(4-methoxy-benzyl)-amino]-thiazole-4-carboxylic acid (2-amino-phenyl)-amide

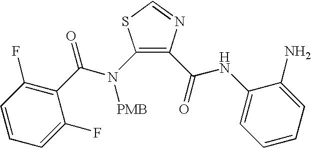

A solution of 5-[(2,6-difluoro-benzoyl)-amino]-thiazole-4-carboxylic acid (500 mg, 1.24 mmoles), o-phenylenediamine (134 mg, 1.24 mmoles), EDC (285 mg, 1.49 mmoles) and HOBt (240 mg, 1.49 mmoles) in DCM (10 ml) was stirred at ambient temperature for 3 hours. The reaction mixture was diluted with EtOAc, and washed with saturated NaHCO₃ solution and then brine. The organic portion was dried (MgSO₄), filtered and evaporated in vacuo. The residue was purified [Biotage SP4, 40S, flow rate 40 ml/min, gradient 3:7 EtOAc/petrol to 7:3 EtOAc/petrol] to give 5-[(2,6-difluoro-benzoyl)-(4-methoxy-benzyl)-amino]-thiazole-4-carboxylic acid (2-amino-phenyl)-amide as a yellow oil (470 mg, 77%). (LC/MS: Rₜ 3.09, [M+H]⁺ 494.98).

### 1E. Synthesis of N-[4-(1 H-benzoimidazol-2-yl)-thiazol-5-yl]-2,6-difluoro-benzamide

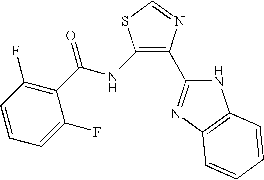

A solution of 5-[(2,6-difluoro-benzoyl)-(4-methoxy-benzyl)-amino]-thiazole-4-carboxylic acid (2-amino-phenyl)-amide (470 mg, 0.95 mmoles) in acetic acid (2 ml) was heated at 120° C. (100 W) in a CEM discover microwave synthesizer for 10 minutes. The reaction mixture was partitioned between EtOAc and sodium hydroxide solution (2N). The organic portion was dried (MgSO₄), filtered and evaporated in vacuo. The residue was dissolved in trifluoroacetic acid (2 ml) and anisole (207 μl) and then heated at 100° C. (80 W) in a CEM discover microwave synthesizer for 10 minutes. The solvent was removed in vacuo. The residue was purified [Biotage SP4, 25M, flow rate 25 ml/min, gradient 3:17 EtOAc/petrol to 3:2 EtOAc/petrol] to give N-[4-(1H-benzoimidazol-2-yl)-thiazol-5-yl]-2,6-difluoro-benzamide as a white solid (200 mg, 59%). (LC/MS: Rₜ 3.34, [M+H]⁺ 356.96).

### EXAMPLE 2

### Synthesis of 2,6-difluoro-N-[4-(6-morpholin-4ylmethyl-1 H-benzoimidazol-2-yl)-thiazol-5-yl]benzamide

### 2A. Synthesis of (3,4-Dinitro-phenyl)-morpholin-4-yl-methanone

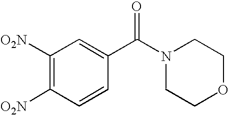

A mixture of 3,4-dinitrobenzoic acid (10.0 g) and thionyl chloride (30 ml) was heated at reflux for 2 hours, cooled to ambient temperature and excess thionyl chloride removed through azeotrope with toluene. The residue was taken up in THF (100 ml) and morpholine (4.1 ml) and Et₃N (7.2 ml) added concurrently to the mixture at 0° C. The mixture was stirred for 3 hours, water (100 ml) added and then extracted with EtOAc. The organic portion was washed with brine, dried (MgSO₄) and reduced in vacuo. Recrystallisation of the residue from MeOH gave (3,4-dinitro-phenyl)-morpholin-4-yl-methanone (8.23 g) as a yellow solid. (¹H NMR (300 MHz, DMSO-d₆) δ 8.3 (d, 1H), 8.3 (s, 1H), 8.0 (d, 1H), 3.7-3.5 (m, 8H)).

### 2B. Synthesis of 4-(3,4-Dinitro-benzyl)-morpholine

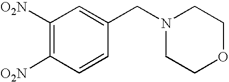

To a mixture of (3,4-dinitro-phenyl)-morpholin-4-yl-methanone (2.84 g) in dry THF (50 ml) was added NaBH₄ (954 mg) followed drop-wise by BF₃.Et₂O (3.2 ml). The mixture was stirred at ambient temperature for 3 hours and then quenched though addition of MeOH. The mixture was reduced in vacuo and partitioned between EtOAc and water. The organic portion washed with brine, dried (MgSO₄) and reduced in vacuo. The residue was purified via flash column chromatography eluting with EtOAc to give 4-(3,4-dinitro-benzyl)-morpholine (1.08 g).

### 2C. Synthesis of 2,6-difluoro-N-[4-(6-morpholin-4ylmethyl-1H-benzoimidazol-2-yl)-thiazol-5-yl]benzamide

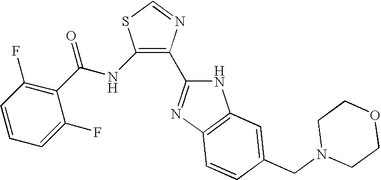

A mixture of 4-(3,4-dinitro-benzyl)-morpholine (1.00 g) and 10% Pd/C (0.10 g) in ethanol (40 ml) was shaken under a hydrogen atmosphere at ambient temperature for 2 hours, diluted with further ethanol (40 ml) and filtered through a plug of Celite, washing with ethanol. The filtrate was reduced in vacuo and triturated with DCM/petroleum ether to give an orange solid (0.789 g), with 4-morpholin-4-ylmethyl-benzene-1,2-diamine as the major component. A sample of this solid (150 mg, 0.77 mmoles), EDC (177 mg, 0.92 mmoles), HOBt (124 mg, 0.92 mmoles) and 5-[(2,6-difluoro-benzoyl)-(4-methoxy-benzyl)-amino]-thiazole-4-carboxylic acid (Example 1C) (311 mg, 0.77 mmoles) was dissolved in DMF (10 ml) and the solution stirred at ambient temperature for 24 hours. The reaction mixture was partitioned between EtOAc and a saturated solution of sodium hydrogen carbonate. The organic portion was dried (MgSO₄), filtered and evaporated in vacuo. The residue was purified by flash chromatography (Biotage SP4, 25M, flow rate 25 ml/min, gradient EtOAc to 1:20 MeOH/EtOAc). The solvent was evaporated in vacuo. The residue was dissolved in acetic acid (2 ml) and the solution formed was heated at 120° C. (100 W) in a CEM discover microwave synthesizer for 10 minutes. The reaction mixture was partitioned between EtOAc and sodium hydroxide solution (2N). The organic portion was dried (MgSO₄), filtered and evaporated in vacuo. The residue and anisole (62 μl, 0.573 mmoles) was dissolved in trifluoroacetic acid and heated at 100° C. (80 W) in a CEM discover microwave synthesizer for 10 minutes. The reaction mixture was azeotroped with toluene in vacuo. The residue was purified by trituration with ether to give 2,6-difluoro-N-[4-(6-morpholin-4ylmethyl-1H-benzoimidazol-2-yl)-thiazol-5-yl]benzamide as a brown solid (55 mg, 42%). (LC/MS: Rₜ 2.19, [M+H]⁺ 456.23).

### EXAMPLE 3

### Synthesis of 2,6-Difluoro-N-[3-(5-morpholin-4-ylmethyl-1H-indol-2-yl)-isothiazol-4-yl]-benzamide

### 3A. Synthesis of 4-Amino-isothiazole-3-carboxylic acid methyl ester

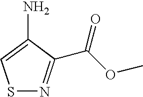

Thionyl chloride (0.620 g, 5.2 mmol) was added dropwise at 0° C. to a solution of 4-amino-isothiazole-3-carboxylic acid (0.500 g, 3.5 mmol) in methanol (10 ml) and the mixture was stirred for 20 hours at ambient temperature. The reaction mixture was reduced in vacuo and dried through azeotrope with toluene to afford 4-amino-isothiazole-3-carboxylic acid methyl ester as a white solid (0.493 g, 90%). (LC/MS: Rₜ 1.60, [M+H]⁺ 159.08).

### 3B. Synthesis of 4-(2,6-Difluoro-benzoylamino)-isothiazole-3-carboxylic acid methyl ester

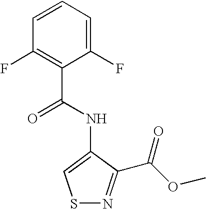

2,6-Difluoro-benzoyl chloride (0.669 g, 3.8 mmol) and triethylamine (0.424 g, 4.2 mmol) were added to a solution of 4-amino-isothiazole-3-carboxylic acid methyl ester (0.493 g, 3.1 mmol) in THF (5 ml) and the resulting suspension was stirred at ambient temperature for 16 hours. The reaction mixture was reduced in vacuo and the residue partitioned between ethyl acetate (50 ml) and water (50 ml) and the aqueous phase back extracted with ethyl acetate (50 ml). The combined organics were washed with brine (50 ml), dried (MgSO₄) and reduced in vacuo. Water (50 ml) was added to the white solid obtained and the resultant suspension basified by the addition of 2M NaOH. The solution was extracted three times with ethyl acetate and the organics were combined, washed (brine), dried (MgSO₄) and reduced in vacuo to give 4-(2,6-difluoro-benzoylamino)-isothiazole-3-carboxylic acid methyl ester as a white solid (0.644 g, 70%). (LC/MS: Rₜ 3.07, [M+H]⁺ 299.14).

### 3C. Synthesis of 4-(2,6-Difluoro-benzoylamino)-isothiazole-3-carboxylic acid

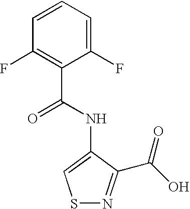

A mixture of 4-(2,6-difluoro-benzoylamino)-isothiazole-3-carboxylic acid methyl ester (0.150 g, 0.5 mmol) in 2 M aqueous NaOH/dioxane (1:1, 6 ml) was stirred at ambient temperature for 16 hours. Volatile materials were removed in vacuo, water (40 ml) was added and the mixture taken to pH 4 by the addition of 2M aqueous HCl. The resultant precipitate was collected by filtration, reduced in vacuo and dried by azeotrope with toluene to give 4-(2,6-fluoro-benzoylamino)-isothiazole-3-carboxylic acid as a white solid (0.103 g, 73%).

### 3D. Synthesis of 2,6-Difluoro-N-[3-(5-morpholin-4-ylmethyl-1H-benzoimidazol-2-yl)-isothiazol -4-yl]-benzamide

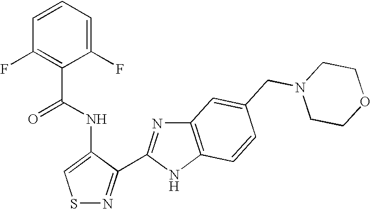

A mixture of 4-(3,4-dinitro-benzyl)-morpholine (Example 2B) (1.00 g) and 10% Pd/C (0.10 g) in ethanol (40 ml) was shaken under a hydrogen atmosphere at ambient temperature for 2 hours, diluted with further ethanol (40 ml) and filtered through a plug of Celite, washing with ethanol. The filtrate was reduced in vacuo and triturated with DCM/petroleum ether to give an orange solid (0.789 g), with 4-morpholin-4-ylmethyl-benzene-1,2-diamine as the major component. A sample of this solid (0.90 g) was added to 4-(2,6-difluoro-benzoylamino)-isothiazole-3-carboxylic acid (0.103 g, 0.36 mmol), EDC (0.085 g, 0.44 mmol), HOBt (0.060 g, 0.44 mmol) and DMF (5 ml) and the resulting reaction mixture was stirred at ambient temperature for 64 hours. The reaction mixture was reduced in vacuo and the residue partitioned between ethyl acetate (50 ml) and saturated aqueous sodium bicarbonate solution (50 ml). The organic layer was washed with brine, dried (MgSO₄), reduced in vacuo to give an orange oil (0.197 g). This oil was taken up in glacial acetic acid (5 ml) and heated at reflux for 3 h. The reaction mixture was then reduced in vacuo and the residue partitioned between ethyl acetate (50 ml) and saturated aqueous sodium bicarbonate solution (50 ml). The organic layer was washed with brine, dried (MgSO₄), reduced in vacuo to give an orange oil (0.161 g), which was subjected to column chromatography, eluting with ethyl acetate, then triturated with diethyl ether and filtered to give 2,6-difluoro-N-[3-(5-morpholin-4-ylmethyl-1H-benzoimidazol-2-yl)-isothiazol-4-yl]-benzamide as a pale yellow solid (0.030 g, 18%). (LC/MS: Rₜ 2.24, [M+H]⁺ 456.22).

### EXAMPLE 4

### 2,3-Dihydro-benzofuran-5-carboxylic acid [4-(6-morpholin-4-ylmethyl-1H-benzoimidazol-2-yl)-thiazol-5-yl]-amide

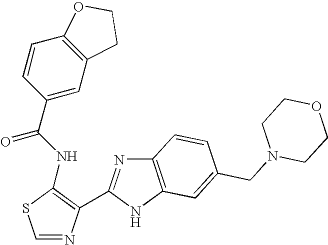

By following the general methods set out in Examples 1 and 2, but substituting 2,3-dihydro-benzofuran-5-carboxylic acid chloride for 2,6-difluorobenzoyl chloride in Example 1B, the title compound can be prepared.

### EXAMPLE 5

### 2-Chloro-4-morpholin-4-yl-N-[4-(6-morpholin-4-ylmethyl-1H-benzoimidazol-2-yl)-thiazol-5-yl]-benzamide

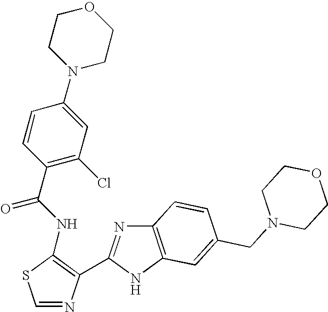

By following the general methods set out in Examples 1 and 2, but substituting 2-chloro-4-morpholin-4-yl-benzoic acid chloride for 2,6-difluorobenzoyl chloride in Example 1B, the title compound can be prepared.

### EXAMPLE 6

### Pyrrolidine-2-carboxylic acid [4-(5,6-dimethoxy-1H-benzoimidazol-2-yl)-thiazol-5-yl]-amide

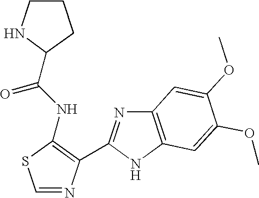

By following the general methods set out in Examples 1 and 2, but substituting 2-pyrrolidinyl-carboxylic acid chloride for 2,6-difluorobenzoyl chloride in Example 1B, and substituting 4,5-dimethoxy-benzene-1,2-diamine for o-phenylene diamine in Example 1D, the title compound can be prepared.

### EXAMPLE 7

### 1-Methyl-piperidine-4-carboxylic acid [4-(5,6-dimethoxy-1H-benzoimidazol-2-yl -thiazol-5-yl]-amide

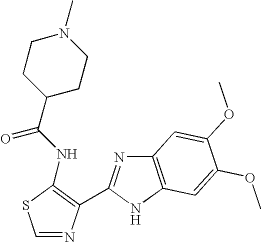

By following the general methods set out in Examples 1 and 2, but substituting 1-methylpiperidin-4-yl-carboxylic acid chloride for 2,6-difluorobenzoyl chloride in Example 1B, and substituting 4,5-dimethoxy-benzene-1,2-diamine for o-phenylene diamine in Example 1D, the title compound can be prepared.

### EXAMPLE 8

### Synthesis of 1-cyclopropyl-3-[3-(5-morpholin-4-ylmethyl-1H-benzoimidazol-2-yl)-thiazol-5-yl]-urea

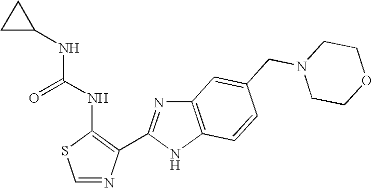

A mixture of 4-(5-morpholin-4-ylmethyl-1H-benzimidazol-2-yl)-1H-thiazole-5-ylamine (0.33 mmol), and CDI (217 mg, 1.34 mmol) in THF (2 ml) is subjected to microwave irradiation (150° C., 150 W) for 15 minutes. Cyclopropylamine (2.68 mmol) is then added and the reaction mixture is irradiated again under identical conditions for a further 15 minutes. After cooling, the heterogeneous mixture is filtered, the filtrate is concentrated and the residue is purified by column chromatography to give the title compound.

The starting material for this preparation, i.e. 4-(5-morpholin-4-ylmethyl-1H-benzimidazol-2-yl)-1H-thiazole-5-ylamine, may be made from a suitably N-protected 5-aminothiazole-4-carboxylic acid using the cyclisation conditions described herein.

Alternatively, 1-cyclopropyl-3-[3-(5-morpholin-4-ylmethyl-1H-benzoimidazol-2-yl)-thiazol-5-yl]-urea can be prepared by a method as described in the general synthesis section herein using reagents and conditions well known to the skilled person.

### EXAMPLE 9

By following the methods described herein, the following compounds may be prepared:

1-(2,6-difluorophenyl)-3-[3-(5-morpholin-4-ylmethyl-1H-benzoimidazol-2-yl)-thiazol-5-yl]-urea;

1-cyclopropyl-3-[3-(5-morpholin-4-ylmethyl-1H-benzoimidazol-2-yl)-isothiazol-4-yl]-urea; and

1-(2,6-difluorophenyl)-3-[3-(5-morpholin-4-ylmethyl-1H-benzoimidazol-2-yl)-isothiazol-4-yl]-urea.

### Biological Activity

### EXAMPLE 10

### Measurement of Activated CDK2/CyclinA Kinase Inhibitory Activity Assay (IC₅₀)

The compounds of the invention can be tested for kinase inhibitory activity using the following protocol.

Activated CDK2/CyclinA (Brown et al, Nat. Cell Biol., 1, pp 438-443, 1999; Lowe, E. D., et al Biochemistry, 41, pp 15625-15634, 2002) is diluted to 125 pM in 2.5× strength assay buffer (50 mM MOPS pH 7.2, 62.5 mM β-glycerophosphate, 12.5 mM EDTA, 37.5 mM MgCl₂, 112.5 mM ATP, 2.5 mM DTT, 2.5 mM sodium orthovanadate, 0.25 mg/ml bovine serum albumin), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 2 to 4 hours before being stopped with an excess of ortho-phosphoric acid (5 μl at 2%).

γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 20 μl of Microscint 20 scintillant is added, and then counted on a Packard Topcount for 30 seconds. The % inhibition of the CDK2 activity is calculated and plotted in order to determine the concentration of test compound required to inhibit 50% of the CDK2 activity (IC₅₀).

The compounds of Examples 1 and 2 each have IC₅₀ values of less than 10 μM in the CDK2 assay.

### EXAMPLE 11

### Measurement of Activated CDK1/CyclinB Kinase Inhibitory Activity Assay (IC₅₀)

CDK1/CyclinB assay.is identical to the CDK2/CyclinA above except that CDK1/CyclinB (Upstate Discovery) is used and the enzyme is diluted to 6.25 nM.

The compounds of Examples 1, 2 and 3 each have IC₅₀ values of less than 10 μM in the CDK2 assay.

### EXAMPLE 12

### Aurora Kinase Assays

Aurora activity was determined using a Dissociative Enhanced Lanthanide Fluoro Immuno Assay (DELFIA) with a GSK3-derived biotinylated peptide. The amount of phosphorylated peptide produced is quantified by means of a phospho-specific primary antibody and europium-labelled anti-rabbit IgG antibody using time-resolved fluorescence at λₑₓ=337 nm, λₑₘ=620 nm.

### Aurora A

### Kinase Reaction

Assay reactions are set up in 96 well plates in a total reaction volume of 25 μl with 0.5 nM AuroraA (Upstate Discovery), 3 μM Biotin-CGPKGPGRRGRRRTSSFAEG, 15 μM ATP and various dilutions of compound in 10 mM MOPS, pH 7.0, 0.1 mg/ml BSA, 0.001% Brij-35, 0.5% glycerol, 0.2 mM EDTA, 10 mM MgCl₂, 0.01% β-mercaptoethanol & 2.5% DMSO. The reaction is allowed to proceed for 60 minutes at room temperature before stopping with 100 μl STOP buffer containing 100 mM EDTA, 0.05% Surfact-Amps20 (Pierce) and 1× Blocker™ BSA in TBS (Pierce).

### Detection Step

The reaction mixture is then transferred to a 96-well Neutravidin-coated plate (Pierce) and incubated for 30 minutes to capture the biotinylated peptide. After washing 5 times with 200 μl TBST buffer per well, a mixture of anti-phospho-(Ser/Thr)-AKT substrate antibody (Cell Signalling Technology) and Eu-N, anti-rabbit IgG (Perkin Elmer) is added to all wells and left for 1 hour. After a further washing step, DELFIA enhancement solution (Perkin Elmer) is added to all wells. After an incubation of 5 minutes, the wells are counted on a Fusion platereader.

In the Aurora A assay, the compounds of Examples 1 to 3 all have IC₅₀ values of less than 0.1 μM.

### Aurora B

### Kinase Reaction

Assay reactions are set up in 96 well plates in a total reaction volume of 25 μl with 5 nM AuroraB (ProQinase), 3 μM Biotin-CGPKGPGRRGRRRTSSFAEG, 15 μM ATP and various dilutions of compound in 25 mM TRIS pH 8.5, 0.1 mg/ml BSA, 0.025% Surfact-Amps 20, 5 mM MgCl₂, 1 mM DTT, & 2.5% DMSO. The reaction is allowed to proceed for 90 minutes at room temperature before stopping with 100 μl STOP buffer containing 100 mM EDTA, 0.05% Surfact-amps20 (Pierce) and 1× Blocker™ BSA in TBS (Pierce).

The detection step was carried out as described for AuroraA.

In the Aurora B assay, the compound of Example 1 was found to have an IC₅₀ value of less than 0.1 μM.

### EXAMPLE 13

### GSK3-B/Aurora Kinase Inhibitory Activity Assay

AuroraA (Upstate Discovery) or GSK3-β (Upstate Discovery) are diluted to 10 nM and 7.5 nM respectively in 25 mM MOPS, pH 7.00, 25 mg/ml BSA, 0.0025% Brij-35, 1.25% glycerol, 0.5 mM EDTA, 25 mM MgCl₂, 0.025% β-mercaptoethanol, 37.5 mM ATP and and 10 μl mixed with 10 μl of substrate mix. The substrate mix for Aurora is 500 μM Kemptide peptide (LRRASLG, Upstate Discovery) in 1 ml of water with 35 μCi γ³³P-ATP. The substrate mix for GSK3-β is 12.5 μM phospho-glycogen synthase peptide-2 (Upstate Discovery) in 1 ml of water with 35 μCi γ³³P-ATP. Enzyme and substrate are added to 96 well plates along with 5 μl of various dilutions of the test compound in DMSO (up to 2.5%). The reaction is allowed to proceed for 30 minutes (Aurora) or 3 hours (GSK3-β) before being stopped with an excess of ortho-phosphoric acid (5 μl at 2%). The filtration procedure is as for Activated CDK2/CyclinA assay above.

### EXAMPLE 14

### CDK Selectivity Assays

Compounds of the invention can be tested for kinase inhibitory activity against a number of different kinases using the general protocol described above, but modified as set out below.

Kinases are diluted to a 10× working stock in 20 mM MOPS pH 7.0, 1 mM EDTA, 0.1% γ-mercaptoethanol, 0.01% Brij-35, 5% glycerol, 1 mg/ml BSA. One unit equals the incorporation of 1 nmol of phosphate per minute into 0.1 mg/ml histone H1, or CDK7 substrate peptide at 30° C. with a final ATP concentration of 100 μM.

The substrate for all the CDK assays (except CDK7) is histone H1, diluted to 10× working stock in 20 mM MOPS pH 7.4 prior to use. The substrate for CDK7 is a specific peptide diluted to 10× working stock in deionised water.

Assay Procedure for CDK1/cyclinB, CDK2/cyclinA, CDK2/cyclinE, CDK3/cyclinE, CDK5/p35, CDK6/cyclinD3:

In a final reaction volume of 25 μl, the enzyme (5-10 mU) is incubated with 8 mM MOPS pH 7.0, 0.2 mM EDTA, 500 μM peptide, 10 mM MgAcetate and [γ-³³P-ATP] (specific activity approx 500 cpm/pmol, concentration as required). The reaction is initiated by the addition of Mg²+[γ-³³P-ATP]. After incubation for 40 minutes at room temperature the reaction is stopped by the addition of 5 μl of a 3% phosphoric acid solution. 10 ml of the reaction is spotted onto a P30 filtermat and washed 3 times for 5 minutes in 75 mM phosphoric acid and once in methanol prior to drying and counting.

Assay procedure for CDK7/cyclinH/MAT1

In a final reaction volume of 25 μl, the enzyme (5-10 mU) is incubated with 8 mM MOPS pH 7.0, 0.2 mM EDTA, 500 μM peptide, 10 mM MgAcetate and [γ-³³P-ATP] (specific activity approx 500 cpm/pmol, concentration as required). The reaction is initiated by the addition of Mg²+[γ-³³P-ATP]. After incubation for 40 minutes at room temperature the reaction is stopped by the addition of 5 μl of a 3% phosphoric acid solution. 10 ml of the reaction is spotted onto a P30 filtermat and washed 3 times for 5 minutes in 75 mM phosphoric acid and once in methanol prior to drying and counting.

### EXAMPLE 15

### Anti-proliferative Activity

The anti-proliferative activities of compounds of the invention can be determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. In addition, any morphological changes are recorded. All cell lines can be obtained from ECACC (European Collection of cell Cultures).

In particular, compounds of the invention were tested against the HCT-116 cell line (ECACC Reference: 91091005) derived from human colon carcinoma.

Thus, the compound of Example 2 was tested against the HCT-116 cell line and was found to have an IC₅₀ of less than 1 μM, whilst the compounds of Examples 1 and 3 both has IC₅₀ values in the same assay of less than 15 μM.

### EXAMPLE 16

### Measurement of inhibitory activity against Glycogen Synthase Kinase-3 (GSK-3)

GSK3β (human) is diluted to a 10× working stock in 50 mM Tris pH 7.5, 0.1 mM EGTA, 0.1 mM sodium vanadate, 0.1% β-mercaptoethanol, 1 mg/ml BSA. One unit equals the incorporation of 1 nmol of phosphate per minute phospho-glycogen synthase peptide 2 per minute.

In a final reaction volume of 25 μl, GSK3β (5-10 mU) is incubated with 8 mM MOPS 7.0, 0.2 mM EDTA, 20 μM YRRAAVPPSPSLSRHSSPHQS(p)EDEEE (phospho GS2 peptide), 10 mM MgAcetate and [γ-³³P-ATP] (specific activity approx 500 cpm/pmol, concentration as required). The reaction is initiated by the addition of Mg²+[γ-³³P-ATP]. After incubation for 40 minutes at room temperature the reaction is stopped by the addition of 5 μl of a 3% phosphoric acid solution. 10 μl of the reaction is spotted onto a P30 filter mat and washed 3 times for 5 minutes in 50 mM phosphoric acid and once in methanol prior to drying and counting.

The compound of Example 1 has an IC₅₀ value of less than 1 μM against GSK3β.

### EXAMPLE 17

### A. General Colony Forming Assay Protocol

The effect of various treatment treatments of compounds on adherent tumour cell lines can be assessed in a clonogenic assay as described below.

Cells are seeded at a concentration of 75 to 100 cells/ml relevant culture media onto 6 or 24 well tissue culture plates and allowed to recover for 16 hours.

Compound or vehicle control (DMSO) is added to duplicate wells to give a final DMSO concentration of 0.1%. Following compound addition, colonies are allowed to grow out for between 10 and 14 days for optimum discrete colony counting. Colonies are fixed in 2 ml Carnoys fixative (25% Acetic Acid, 75% Methanol) and stained in 2 ml 0.4% w/v crystal violet. The number of colonies in each well is counted. IC₅₀ values are calculated by sigmoidal dose-response (variable slope) IC₅₀ curves using Prism Graphpad Software.

By way of example, the effect of various treatments of a compound of the formula (I) on A2780, A549, HCT 116, HCT 116 N7, HT-29, MCF7, MIA-Pa-Ca-2, SW620 cell lines can be assessed in a clonogenic assay.

Cells are seeded at a concentration of 75 to 100 cells/ml relevant culture media onto 6 or 24 well tissue culture plates and allowed to recover for 16 hours.

| Cell Line | Media | Comments |
|---|---|---|
| HCT 116 | DMEM + 10% FBS + GLUTAMAX I | |
| HCT 116 N7 | DMEM + 10% FBS + GLUTAMAX I + | |
| | 0.4 mg/ml G418 | |
| HT-29 | McCoy'5a + 10% FBS + 2 mM L-Glutamine | |
| SW620 | L-15 + 10% FBS + GLUTAMAX I | Atmospheric |
| | | CO₂ |
| A2780 | RPMI 1640 + 2 mM Glutamine + 10% FBS | |
| A549 | DMEM + 10% FBS + GLUTAMAX I | |
| MCF7 | EMEM + 10% FBS + 2 mM L-Glutamine + | |
| | 1% NEAA | |
| MIA- | DMEM + 10% FBS + GLUTAMAX I | |
| Pa-Ca-2 | | |

A compound of formula (I) or vehicle control (DMSO) is added to duplicate wells to give a final DMSO concentration of 0.1%. Following compound addition, colonies are allowed to grow out for between 10 and 14 days for optimum discrete colony counting. Colonies are fixed in 2 ml Camoys fixative (25% Acetic Acid, 75% Methanol) and stained in 2 ml 0.4% w/v crystal violet. The number of colonies in each well is counted. IC₅₀ values are calculated by sigmoidal dose-response (variable slope) IC₅₀ curves using Prism Graphpad Software.

### Pharmaceutical Formulations

### EXAMPLE 18

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### (v) Injectable formulation III

A formulation for i.v. delivery by injection or infusion can be prepared by dissolving the compound of formula (I) (e.g. in a salt form) in water at 20 mg/ml. The vial is then sealed and sterilised by autoclaving.

### (vi) Injectable formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### (viii) Lyophilised Formulation

Aliquots of formulated compound of formula (I) or a salt thereof as defined herein are put into 50 mL vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EXAMPLE 19

### Determination of Antifungal Activity

The antifungal activity of the compounds of the formula (I) is determined using the following protocol.

The compounds are tested against a panel of fungi including Candida parpsilosis, Candida tropicalis, Candida albicans-ATCC 36082 and Cryptococcus neoformans. The test organisms are maintained on Sabourahd Dextrose Agar slants at 4° C. Singlet suspensions of each organism are prepared by growing the yeast overnight at 27° C. on a rotating drum in yeast-nitrogen base broth (YNB) with amino acids (Difco, Detroit, Mich.), pH 7.0 with 0.05 M morpholine propanesulphonic acid (MOPS). The suspension is then centrifuged and washed twice with 0.85% NaCl before sonicating the washed cell suspension for 4 seconds (Branson Sonifier, model 350, Danbury, Conn.). The singlet blastospores are counted in a haemocytometer and adjusted to the desired concentration in 0.85% NaCl.

The activity of the test compounds is determined using a modification of a broth microdilution technique. Test compounds are diluted in DMSO to a 1.0 mg/ml ratio then diluted to 64 μg/ml in YNB broth, pH 7.0 with MOPS (Fluconazole is used as the control) to provide a working solution of each compound. Using a 96-well plate, wells 1 and 3 through 12 are prepared with YNB broth, ten fold dilutions of the compound solution are made in wells 2 to 11 (concentration ranges are 64 to 0.125 μg/ml). Well 1 serves as a sterility control and blank for the spectrophotometric assays. Well 12 serves as a growth control. The microtitre plates are inoculated with 10 μl in each of well 2 to 11 (final inoculum size is 10⁴ organisms/ml). Inoculated plates are incubated for 48 hours at 35° C. The IC50 values are determined spectrophotometrically by measuring the absorbance at 420 nm (Automatic Microplate Reader, DuPont Instruments, Wilmington, Del.) after agitation of the plates for 2 minutes with a vortex-mixer (Vorte-Genie 2 Mixer, Scientific Industries, Inc., Bolemia, N.Y.). The IC50 endpoint is defined as the lowest drug concentration exhibiting approximately 50% (or more) reduction of the growth compared with the control well. With the turbidity assay this is defined as the lowest drug concentration at which turbidity in the well is <50% of the control (IC50). Minimal Cytolytic Concentrations (MCC) are determined by sub-culturing all wells from the 96-well plate onto a Sabourahd Dextrose Agar (SDA) plate, incubating for 1 to 2 days at 35° C. and then checking viability.

### EXAMPLE 20

### Protocol for the Biological Evaluation of Control of in vivo Whole Plant Fungal Infection

Compounds of the formula (I) are dissolved in acetone, with subsequent serial dilutions in acetone to obtain a range of desired concentrations. Final treatment volumes are obtained by adding 9 volumes of 0.05% aqueous Tween-20™ or 0.01% Triton X-100™, depending upon the pathogen.

The compositions are then used to test the activity of the compounds of the invention against tomato blight (Phytophthora infestans) using the following protocol. Tomatoes (cultivar Rutgers) are grown from seed in a soil-less peat-based potting mixture until the seedlings are 10-20 cm tall. The plants are then sprayed to run-off with the test compound at a rate of 100 ppm. After 24 hours the test plants are inoculated by spraying with an aqueous sporangia suspension of Phytophthora infestans, and kept in a dew chamber overnight. The plants are then transferred to the greenhouse until disease develops on the untreated control plants.

Similar protocols are also used to test the activity of the compounds of the invention in combatting Brown Rust of Wheat (Puccinia), Powdery Mildew of Wheat (Ervsiphe vraminis), Wheat (cultivar Monon), Leaf Blotch of Wheat (Septoria tritici), and Glume Blotch of Wheat (Leptosphaeria nodorum).

### Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-108. (canceled)

109. A compound of the formula (I): 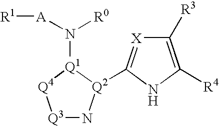
or a salt, N-oxide, tautomer or solvate thereof;
wherein
X is CR⁵ or N;
each of Q¹ and Q² is a carbon atom;
Q³ is selected from S and CH;
Q⁴ is selected from CR² and S; provided that one of Q³ and Q⁴ is S and the other of Q³ and Q⁴ is not S;
wherein when Q³ is S, there is a double bond between Q¹ and Q⁴ and a double bond between Q² and the adjacent ring nitrogen atom N; and when Q⁴ is S, there is a double bond between Q¹ and Q², and a double bond between Q³ and the adjacent ring nitrogen atom N;
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³ and R⁴ together with the carbon atoms to which they are attached form an optionally substituted fused carbocyclic or heterocyclic ring having from 5 to 7 ring members of which up to 3 can be hetero atoms selected from N, O and S; and
R⁵ is hydrogen, a group R² or a group R¹⁰ wherein R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbo cyclic and hetero cyclic groups having from 3 to 12 ring members, and a C₁₋₈ hydrocarbyl group optionally substituted by one or more substituents selected from hydroxy, oxo, halogen, cyano, nitro, carboxy, armino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

110. A compound according to claim 109 wherein Q³ is S and Q⁴ is CR² and hence the compound of the formula (I) is an isothiazole.

111. A compound according to claim 109 wherein R² is hydrogen.

112. A compound according to claim 109 wherein Q³ is CH and Q⁴ is S and hence the compound of the formula (I) is a thiazole.

113. A compound according to claim 109 wherein X is N.

114. A compound according to claim 109 wherein R⁰ is hydrogen.

115. A compound according to claim 109 wherein the moiety R¹-A-NH linked to the moiety Q¹ takes the form of an amide R¹—(CH₂)ₘ—C(═O)NH or a urea R¹—(CH₂)ₘ—NHC(═O)NH wherein in each case m is 0, 1 or 2.

116. A compound according to claim 115 wherein the moiety R¹-A-NH linked to the moiety Q¹ takes the form of an amide R¹—(CH₂)ₘ—C(═O)NH.

117. A compound according to claim 115 wherein the moiety R¹-A-NH linked to the moiety Q¹ takes the form of a urea R¹—(CH₂)ₘ—NHC(═O)NH.

118. A compound according to claim 109 wherein R¹ is a monocyclic or bicyclic group having from 3 to 10 ring members.

119. A compound according to claim 118 wherein R¹ is selected from unsubstituted and substituted phenyl, pyrazolo[1,5-a]pyridinyl, 2,3-dihydro-benzo[1,4]dioxine, indol-4-yl, 2,3-dihydrobenzofuranyl, tert-butyl, furanyl, pyrazolo[1,5-a]pyridin-3-yl, pyrazolo[1,5-a]pyrimidin-3-yl, oxazolyl, isoxazolyl, benzoxazol-2-yl, 2H-tetrazol-5-yl pyrazin-2-yl, pyrazolyl benzyl, α,α-dimethylbenzyl, α-aminobenzyl, α-methylaminobenzyl, 4,5,6,7-tetrahydro-benzo[d]isoxazol-3-yl, 2H-phthalazin-1-one-4-yl, benzoxazol-7-yl, quinazolinyl, 2-naphthyl, cyclopropyl, benzo[c]isoxazol-3-yl, 4-piperidinyl, 5-thiazolyl, 2-pyridyl, 3-pyridyl, 3-pyrrolyl, isoxazolyl, imidazo[2,1-b]thiazolyl, 4-pyrimidinyl, cyclohexyl, tetrahydropyran-4-yl, tetrahydroquinolinyl, 4,5,6,7-tetrahydro-benzofuranyl and morpholinyl groups; wherein one or more substituents R¹⁰ can be present and are selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic carbocyclic or heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c}.

120. A compound according to claim 119 wherein the substituents on R¹ are selected from the group R^{10a} consisting of halogen, hydroxy, trifluoromethyl, cyano, nitro, carboxy, heterocyclic groups having 5 or 6 ring members and up to 2 heteroatoms selected from O, N and S, a group R^{a}-R^{b} wherein R^{a} is a bond, O, CO, X³C(X⁴), C(X)X³, X³C(X⁴)X³, S, SO, or SO₂, and R^{b} is selected from hydrogen, heterocyclic groups having 5 or 6 ring members and up to 2 heteroatoms selected from O, N and S, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having 5 or 6 ring members and up to 2 heteroatoms selected from O, N and S; wherein one or more carbon atoms of the C₁₋₈ hydrocarbyl group may optionally be replaced by O, S, SO, SO₂, X³C(X⁴), C(X⁴)X³ or X³C(X)X³; X³ is O or S; and X⁴ is ═O or ═S.

121. A compound according to claim 119 wherein R¹ bears 1 or 2 or 3 substituents selected from fluorine, chlorine, methoxy, ethoxy, methyl, ethyl, isopropyl, tert-butyl, amino, oxazolyl, morpholino, trifluoromethyl, bromomethyl, chloroethyl, pyrrolidino, pyrrolidinylethoxy, pyrrolidinylmethyl, difluoromethoxy, trifluoromethoxy, morpholino, N-methylpiperazino, piperazine, piperidino, pyrrolidino, and morpholinomethyl.

122. A compound according to claim 109 wherein R¹ is selected from 2,6-difluorophenyl, 2-methoxyphenyl, 2,6-difluoro-4-methoxyphenyl, 2-fluoro-6-methoxyphenyl, 2-fluoro-5-methoxyphenyl, 2,6-dimethoxyphenyl, 2,4-dimethoxyphenyl, 2-chloro-6-fluorophenyl, 2,6-dichlorophenyl, 2,4,6-trifluorophenyl, 2-chloro-6-methyl, 2,3-dihydro-benzo[1,4]dioxin-5-yl and pyrazolo[1,5-a]pyridin-3-yl.

123. A compound according to claim 122 wherein R¹ is 2,6-difluorophenyl.

124. A compound according to claim 119 wherein R¹ is cyclopropyl.

125. A compound according to claim 119 wherein R³ and R⁴ together with the five membered ring to which they are attached form an optionally substituted ring system selected from ring systems (i) to (iv): 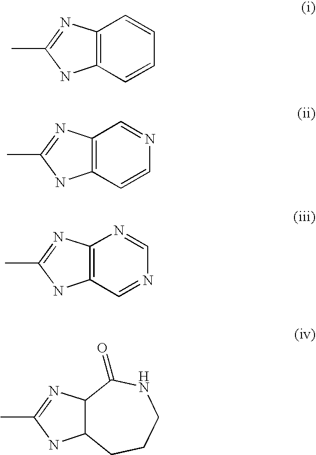
wherein each ring system is optionally substituted by one or more groups R¹⁰ as defined in claim 119.

126. A compound according to claim 125 wherein the ring system is ring system (i).

127. A compound according to claim 125 wherein the substituent groups R¹⁰ are selected from halogen, a group R^{a}-R^{b} wherein R^{a} is a bond, O, CO, C(X²)X¹, and R^{b} is selected from hydrogen, heterocyclic groups having 3-7 ring members and a C₁₋₄ hydrocarbyl group optionally substituted by one or more substituents selected from hydroxy, carboxy, amino, mono- or di-C₁₋₄ hydrocarbylamino, and heterocyclic groups with 3-7 ring members.

128. A compound according to claim 109 having the formula (II): 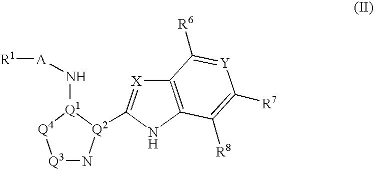
wherein Q¹-Q⁴, R¹, R² and X are as defined in claim 109;
Y is N or CR⁹ wherein R⁹ is hydrogen or a group R¹⁰; and R⁶, R⁷ and R⁸ are the same or different and each is hydrogen or a group R¹⁰ as defined in claim 109.

129. A compound according to claim 128 having the formula (III):

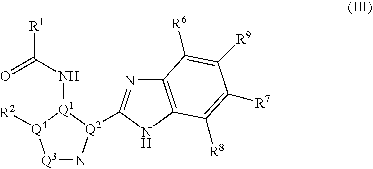

130. A compound according to claim 128 having the formula (IIIa):

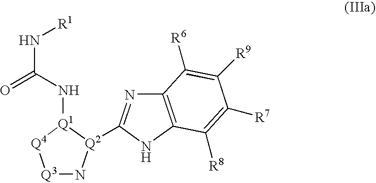

131. A compound according to claim 109 having the formula (IV): 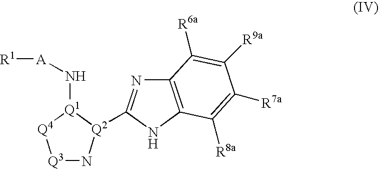
wherein A is NH(C═O), O(C═O) or C═O;
R² and Q¹ to Q⁴ are as defined in claim 109;
R^{6a}, R^{7a}, R^{8a} and R^{9a} are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R^{6a}, R^{7a}, R^{8a} or R^{9a} together with the carbon atoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c};
or an adjacent pair of substituents selected from R^{6a}, R^{7a}, R^{8a} and R^{9a} together with the carbon atoms to which they are attached may form a non-aromatic five or six membered ring containing up to three heteroatoms selected from O, N and S;
R^{1a} is selected from:
6-membered monocyclic aryl groups substituted by one to three substituents R^{10c} provided that when the aryl group is substituted by a methyl group, at least one substituent other than methyl is present;
6-membered monocyclic heteroaryl groups containing a single heteroatom ring member which is nitrogen, the heteroaryl groups being substituted by one to three substituents R^{10c};
5-membered monocyclic heteroaryl groups containing up to three heteroatom ring members selected from nitrogen and sulphur, and being optionally substituted by one to three substituents R^{10c};
5-membered monocyclic heteroaryl groups containing a single oxygen heteroatom ring member and optionally a nitrogen heteroatom ring member, and being substituted by one to three substituents R^{10c} provided that when the heteroaryl group contains a nitrogen ring member and is substituted by a methyl group, at least one substituent other than methyl is present;
bicyclic aryl and heteroaryl groups having up to four heteroatom ring members and wherein either one ring is aromatic and the other ring is non-aromatic, or wherein both rings are aromatic, the bicyclic groups being optionally substituted by one to three substituents R^{10c};
four-membered, six-membered and seven-membered monocyclic C-linked saturated heterocyclic groups containing up to three heteroatoms selected from nitrogen, oxygen and sulphur, the heterocyclic groups being optionally substituted by one to three substituents R^{10c} provided that when the heterocyclic group has six ring members and contains only one heteroatom which is oxygen, at least one substituent R^{10c} is present;
five membered monocyclic C-linked saturated heterocyclic groups containing up to three heteroatoms selected from nitrogen, oxygen and sulphur, the heterocyclic groups being optionally substituted by one to three substituents R^{10c} provided that when the heterocyclic group has five ring members and contains only one heteroatom which is nitrogen, at least one substituent R^{10c} other than hydroxy is present;
four and six membered cycloalkyl groups optionally substituted by one to three substituents R^{10c};
three and five membered cycloalkyl groups substituted by one to three substituents R^{10c}; and
a group Ph′CR¹⁷R¹⁸— where Ph′ is a phenyl group substituted by one to three substituents R^{10c}; R¹⁷ and R¹⁸ are the same or different and each is selected from hydrogen and methyl; or R¹⁷ and R¹⁸ together with the carbon atom to which they are attached form a cyclopropyl group; or one of R¹⁷ and R¹⁸ is hydrogen and the other is selected from amino, methylamino, C₁₋₄ acylamino, and C₁₋₄ alkoxycarbonylamino;
and where one of R^{6a}, R^{7a}, R^{8a} and R^{9a} is a morpholinomethyl group, then R^{1a} is additionally selected from:
unsubstituted phenyl and phenyl substituted with one or more methyl groups;
unsubstituted 6-membered monocyclic heteroaryl groups containing a single heteroatom ring member which is nitrogen;
unsubstituted furyl;
5-membered monocyclic heteroaryl groups containing a single oxygen heteroatom ring member and a nitrogen heteroatom ring member, and being unsubstituted or substituted by one or more methyl groups;
unsubstituted six membered monocyclic C-linked saturated heterocyclic groups containing only one heteroatom which is oxygen; and
unsubstituted three and five membered cycloalkyl groups;
and R^{10c} is selected from:
halogen (e.g. F and Cl);
hydroxyl;
C₁₋₄ hydrocarbyloxy optionally substituted by one or more substituents selected from hydroxyl and halogen;
C¹⁻⁴ hydrocarbyl substituted by one or more substituents selected from hydroxyl, halogen and five and six-membered saturated heterocyclic rings containing one or two heteroatom ring members selected from nitrogen, oxygen and sulphur;
S—C₁₋₄ hydrocarbyl;
phenyl optionally substituted with one to three substituents selected from C₁₋₄ alkyl, trifluoromethyl, fluoro and chloro;
heteroaryl groups having 5 or 6 ring members (e.g. oxazole, pyridyl, pyrimidinyl) and containing up to 3 heteroatoms selected from N, O and S, the heteroaryl groups being optionally substituted with one to three substituents selected from C₁₋₄ alkyl, trifluoromethyl, fluoro and chloro;
5- and 6-membered non-aromatic heterocyclic groups (e.g. pyrrolidino, piperidino, piperazine, N-methylpiperazino, morpholino) containing up to 3 heteroatoms selected from N, O and S and being optionally substituted with one to three substituents selected from C₁₋₄ alkyl, trifluoromethyl, fluoro and chloro;
cyano, nitro, amino, C₁₋₄ alkylamino, di-C₁₋₄alkylamino, C₁₋₄ acylamino, C₁₋₄ alkoxycarbonylamino;
a group R¹⁹—S(O)ₙ— where n is 0, 1 or 2 and R¹⁹ is selected from amino; C₁₋₄ alkylamino; di-C₁₋₄alkylamino; C₁₋₄ hydrocarbyl; phenyl optionally substituted with one to three substituents selected from C₁₋₄ alkyl, trifluoromethyl, fluoro and chloro; and 5- and 6-membered non-aromatic heterocyclic groups containing up to 3 heteroatoms selected from N, O and S and being optionally substituted with one to three C₁₋₄ alkyl group substituents; and
a group R²⁰-Q- where R²⁰ is phenyl optionally substituted with one to three substituents selected from C₁₋₄ alkyl, trifluoromethyl, fluoro and chloro; and Q is a linker group selected from OCH₂, CH₂O, NH, CH₂NH, NCH₂, CH₂, NHCO and CONH.

132. A compound according to claim 109 having the formula (V): 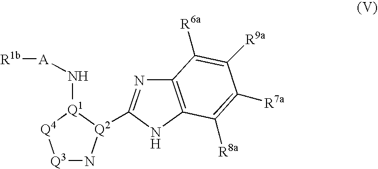
wherein
A is NH(C═O) or C═O;
R² and Q¹ to Q⁴ are as defined in claim 109;
R^{1b} is a substituted phenyl group having from 1 to 4 substituents whereby:
(i) when R^{1b} bears a single substituent it is selected from halogen, hydroxyl, C₁₋₄ hydrocarbyloxy optionally substituted by one or more substituents selected from hydroxyl and halogen; C₁₋₄ hydrocarbyl substituted by one or more substituents selected from hydroxyl and halogen; heteroaryl groups having 5 ring members; and 5- and 6-membered non-aromatic heterocyclic groups, wherein the heteroaryl and heterocyclic groups contain up to 3 heteroatoms selected from N, O and S;
(ii) when R^{1b} bears 2, 3 or 4 substituents, each is selected from halogen, hydroxyl, C₁₋₄ hydrocarbyloxy optionally substituted by one or more substituents selected from hydroxyl and halogen; C₁₋₄ hydrocarbyl optionally substituted by one or more substituents selected from hydroxyl and halogen; heteroaryl groups having 5 ring members; amino; and 5- and 6-membered non-aromatic heterocyclic groups; or two adjacent substituents together with the carbon atoms to which they are attached form a 5-membered heteroaryl ring or a 5- or 6-membered non-aromatic heterocyclic ring; wherein the said heteroaryl and heterocyclic groups contain up to 3 heteroatoms selected from N, O and S; and R^{6a}, R^{7a}, R^{8a} and R^{9a} are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R^{6a}, R^{7a}, R^{8a} or R^{9a} together with the carbon atoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c}; or an adjacent pair of substituents selected from R^{6a}, R^{7a}, R^{8a} and R^{9a} together with the carbon atoms to which they are attached may form a non-aromatic five or six membered ring containing up to three heteroatoms selected from O, N and S.

133. A compound according to claim 132 wherein the compound of the formula (V) is represented by the formula (Va): 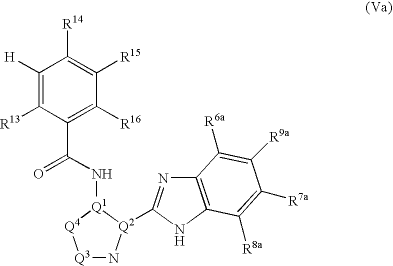
wherein
(i) R¹³ is methoxy and R¹⁴ to R¹⁶ each are hydrogen; or
(ii) R¹⁴ is oxazolyl, imidazolyl or thiazolyl, and R¹³, R¹⁵ and R¹⁶ each are hydrogen; or
(iii) R¹³ is selected from fluorine, chlorine and methyl, R¹⁶ is selected from fluorine, chlorine, methyl and methoxy, and R¹⁴ and R¹⁵ each are hydrogen; or
(iv) R¹³ and R¹⁶ each are selected from fluorine, chlorine and methyl; R¹⁴ is selected from fluorine, chlorine, methyl and methoxy; and R¹⁵ is hydrogen; or
(v) R¹³ and R¹⁴ each are hydrogen; R¹⁵ is selected from fluorine, chlorine, methyl and methoxy, and R¹⁶ is selected from fluorine, chlorine and methyl, or R¹⁵ and R¹⁶ together with the carbon atoms of the phenyl ring form a group selected from:

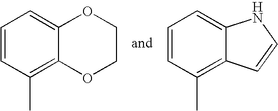

134. A compound according to claim 109 having the formula (VI): 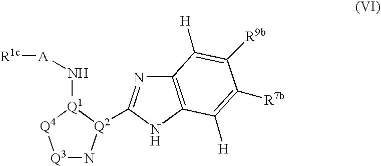
wherein:
Q¹-Q⁴ are as defined in claim 109;
when A is NH(C═O) or C═O;
R^{1c} is selected from:
(a) a mono-substituted phenyl group wherein the substituent is selected from o-amino, o-methoxy; o-chloro;p-chloro; o-difluoromethoxy; o-trifluoromethoxy; o-tert-butyloxy; m-methylsulphonyl andp-fluoro;
(b) a 2,4- or 2,6-disubstituted phenyl group wherein one substituent is selected from o-methoxy, o-ethoxy, o-fluoro, p-morpholino and the other substituent is selected from o-fluoro, o-chloro, p-chloro, and p-amino;
(c) a 2,5-disubstituted phenyl group wherein one substituent is selected from o-fluoro and o-methoxy and the other substituent is selected from m-methoxy, m-isopropyl; m-fluoro, m-trifluoromethoxy, m-trifluoromethyl, m-methylsulphanyl, m-pyrrolidinosulphonyl, m-(4-methylpiperazin-1-yl)sulphonyl, m-morpholinosulphonyl, m-methyl, m-chloro and m-aminosulphonyl;
(d) a 2,4,6-tri-substituted phenyl group where the substituents are the same or different and are each selected from o-methoxy, o-fluoro, p-fluoro, p-methoxy provided that no more than one methoxy substituent is present;
(e) a 2,4,5-tri-substituted phenyl group where the substituents are the same or different and are each selected from o-methoxy, m-chloro and p-amino;
(f) unsubstituted benzyl; 2,6-difluorobenzyl; α,α-dimethylbenzyl; 1-phenylcycloprop-1-yl; and α-tert-butoxycarbonylaminobenzyl;
(g) an unsubstituted 2-furyl group or a 2-furyl group bearing a single substituent selected from 4-(morpholin-4-ylmethyl), piperidinylmethyl; and optionally a further substituent selected from methyl;
(h) an unsubstituted pyrazolo[1,5-a]pyridin-3-yl group;
(i) isoxazolyl substituted by one or two C₁₋₄ alkyl groups;
(j) 4,5,6,7-tetrahydro-benzo[d]isoxazol-3-yl;
(k) 3-tert-butyl-phenyl-1H-pyrazol-5-yl;
(l) quioxalinyl;
(m) benzo[c]isoxazol-3-yl;
(n) 2-methyl-4-trifluoromethyl-thiazol-5-yl;
(o) 3-phenylamino-2-pyridyl;
(p) 1-toluenesulphonylpyrrol-3-yl;
(q) 2,4-dimethoxy-3-pyridyl; and 6-chloro-2-methoxy-4-methyl-3-pyridyl;
(r) imidazo[2,1-b]thiazol-6-yl;
(s) 5-chloro-2-methylsulphanyl-pyrimidin-4-yl;
(t) 3-methoxy-naphth-2-yl;
(u) 2,3-dihydro-benzo[1,4]dioxin-5-yl;
(v) 2,3-dihydro-benzofuranyl group optionally substituted in the five membered ring by one or two methyl groups;
(w) 2-methyl-benzoxazol-7-yl;
(x) 4-aminocyclohex-1-yl;
(y) 1,2,3,4-tetrahydro-quinolin-6-yl;
(z) 2-methyl-4,5,6,7-tetrahydro-benzofuran3-yl;
(aa) 2-pyrimidinyl-lpiperidin-4-yl; and 1-(5-trifluoromethyl-2-pyridyl)-piperidin-4-yl and 1-methylsulphonylpiperidin-4-yl;
(ab) 1-cyanocyclopropyl;
(ac) N-benzylmorpholin-2-yl;
and when A is NH(C=O), R¹ is additionally selected from:
(ad) unsubstituted phenyl;
R^{9b} is selected from hydrogen; chlorine; methoxy; methylsulphonyl; 4-methyl-piperazin-1-ylcarbonyl; morpholinocarbonyl; morpholinomethyl; pyrrolidinylcarbonyl; N-methyl-piperidinyloxy; pyrrolidinylethoxy; morpholinopropylaminomethyl; 4-cyclopentyl-piperazin-1-ylmethyl; 4-ethylsulphonyl-piperazin-1-ylmethyl; morpholinosulphonyl; 4-(4-methylcyclohexyl)-piperazin-1-ylmethyl; and
R^{7b} is selected from hydrogen; methyl; methoxy and ethoxy.

135. A compound according to claim 109 having the formula (VII): 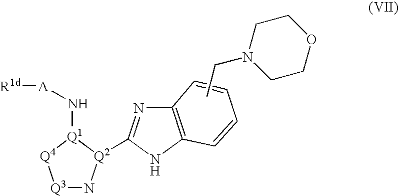
wherein R^{1d} is a group R¹ as defined in claim 109, and R² and Q¹ to Q⁴ are as defined in claim 109.

136. A compound according to claim 135 which is represented by formula (VIIa): 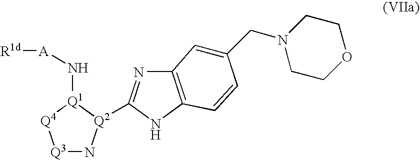
wherein A is NH(C═O) and R^{1d} is cyclopropyl or 2,6-difluorophenyl.

137. A compound according to claim 109 which is represented by formula (VIII): 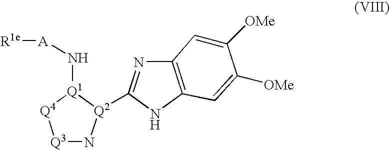
where R^{1e} is a group R^{1a} wherein R^{1a} is selected from:
6-membered monocyclic aryl groups substituted by one to three substituents R^{10c} provided that when the aryl group is substituted by a methyl group, at least one substituent other than methyl is present;
6-membered monocyclic heteroaryl groups containing a single heteroatom ring member which is nitrogen, the heteroaryl groups being substituted by one to three substituents R^{10c};
5-membered monocyclic heteroaryl groups containing up to three heteroatom ring members selected from nitrogen and sulphur, and being optionally substituted by one to three substituents R^{10c};
5-membered monocyclic heteroaryl groups containing a single oxygen heteroatom ring member and optionally a nitrogen heteroatom ring member, and being substituted by one to three substituents R^{10c} provided that when the heteroaryl group contains a nitrogen ring member and is substituted by a methyl group, at least one substituent other than methyl is present;
bicyclic aryl and heteroaryl groups having up to four heteroatom ring members and wherein either one ring is aromatic and the other ring is non-aromatic, or wherein both rings are aromatic, the bicyclic groups being optionally substituted by one to three substituents R^{10c};
four-membered, six-membered and seven-membered monocyclic C-linked saturated heterocyclic groups containing up to three heteroatoms selected from nitrogen, oxygen and sulphur, the heterocyclic groups being optionally substituted by one to three substituents R^{10c} provided that when the heterocyclic group has six ring members and contains only one heteroatom which is oxygen, at least one substituent R^{10c} is present;
five membered monocyclic C-linked saturated heterocyclic groups containing up to three heteroatoms selected from nitrogen, oxygen and sulphur, the heterocyclic groups being optionally substituted by one to three substituents R^{10c} provided that when the heterocyclic group has five ring members and contains only one heteroatom which is nitrogen, at least one substituent R^{10c} other than hydroxy is present;
four and six membered cycloalkyl groups optionally substituted by one to three substituents R^{10c};
three and five membered cycloalkyl groups substituted by one to three substituents R^{10c}; and
a group Ph′CR¹⁷R¹⁸— where Ph′ is a phenyl group substituted by one to three substituents R^{10c}; R¹⁷ and R¹⁸ are the same or different and each is selected from hydrogen and methyl; or R¹⁷ and R¹⁸ together with the carbon atom to which they are attached form a cyclopropyl group; or one of R¹⁷ and R¹⁸ is hydrogen and the other is selected from amino, methylamino, C₁₋₄ acylamino, and C₁₋₄ alkoxycarbonylamino;
and where one of R^{6a}, R^{7a}, R^{8a} and R^{9a} is a morpholinomethyl group, then R^{1a} is additionally selected from:
unsubstituted phenyl and phenyl substituted with one or more methyl groups;
unsubstituted 6-membered monocyclic heteroaryl groups containing a single heteroatom ring member which is nitrogen;
unsubstituted furyl;
5-membered monocyclic heteroaryl groups containing a single oxygen heteroatom ring member and a nitrogen heteroatom ring member, and being unsubstituted or substituted by one or more methyl groups;
unsubstituted six membered monocyclic C-linked saturated heterocyclic groups containing only one heteroatom which is oxygen; and
unsubstituted three and five membered cycloalkyl groups;
and R^{10c} is selected from:
halogen;
hydroxyl;
C₁₋₄ hydrocarbyloxy optionally substituted by one or more substituents selected from hydroxyl and halogen;
C₁₋₄ hydrocarbyl substituted by one or more substituents selected from hydroxyl, halogen and five and six-membered saturated heterocyclic rings containing one or two heteroatom ring members selected from nitrogen, oxygen and sulphur;
S—C₁₋₄ hydrocarbyl;
phenyl optionally substituted with one to three substituents selected from C₁₋₄ alkyl, trifluoromethyl, fluoro and chloro;
heteroaryl groups having 5 or 6 ring members (e.g. oxazole, pyridyl, pyrimidinyl) and containing up to 3 heteroatoms selected from N, O and S, the heteroaryl groups being optionally substituted with one to three substituents selected from C₁₋₄ alkyl, trifluoromethyl, fluoro and chloro;
5- and 6-membered non-aromatic heterocyclic groups (e.g. pyrrolidino, piperidino, piperazine, N-methylpiperazino, morpholino) containing up to 3 heteroatoms selected from N, O and S and being optionally substituted with one to three substituents selected from C₁₋₄ alkyl, trifluoromethyl, fluoro and chloro;
cyano, nitro, amino, C₁₋₄ alkylamino, di-C₁₋₄ alkylamino, C₁₋₄ acylamino, C₁₋₄ alkoxycarbonylamino;
a group R¹⁹—S(O)ₙ— where n is 0, 1 or 2 and R¹⁹ is selected from amino; C₁₋₄ alkylamino; di-C₁₋₄alkylamino; C₁₋₄ hydrocarbyl; phenyl optionally substituted with one to three substituents selected from C₁₋₄ alkyl, trifluoromethyl, fluoro and chloro; and 5- and 6-membered non-aromatic heterocyclic groups containing up to 3 heteroatoms selected from N, O and S and being optionally substituted with one to three C₁₋₄ alkyl group substituents; and
a group R²⁰-Q- where R²⁰ is phenyl optionally substituted with one to three substituents selected from C₁₋₄ alkyl, trifluoromethyl, fluoro and chloro; and Q is a linker group selected from OCH₂, CH₂O, NH, CH₂NH, NCH₂, CH₂, NHCO and CONH.

138. A compound according to claim 109 which is:
N-[4-(1H-benzoimidazol-2-yl)-thiazol-5-yl]-2,6-difluoro-benzamide;
2,6-difluoro-N-[4-(6-morpholin-4-ylmethyl-1H-benzoimidazol-2-yl)-thiazol-5-yl]benzamide;
2,6-difluoro-N-[3-(5-morpholin-4-ylmethyl-1H-indol-2-yl)-isothiazol-4-yl]-benzamide;
2,3-dihydro-benzofuran-5-carboxylic acid [4-(6-morpholin-4-ylmethyl-1H-benzoimidazol-2-yl)-thiazol-5-yl]-amide;
2-chloro-4-morpholin-4-yl-N-[4-(6-morpholin-4-ylmethyl-1H-benzoimidazol-2-yl)-thiazol-5-yl]-benzamide;
pyrrolidine-2-carboxylic acid [4-(5,6-dimethoxy-1H-benzoimidazol-2-yl)-thiazol-5-yl]-amide;
1-methyl-piperidine-4-carboxylic acid [4-(5,6-dimethoxy-1H-benzoimidazol-2-yl)-thiazol-5-yl]-amide; and
1-cyclopropyl-3-[3-(5-morpholin-4-ylmethyl-1H-benzoimidazol-2-yl)-thiazol-5-yl]-urea;
1-(2,6-difluorophenyl)-3-[3-(5-morpholin-4-ylmethyl-1H-benzoimidazol-2-yl)-thiazol-5-yl]-urea;
1-cyclopropyl-3-[3-(5-morpholin-4-ylmethyl-1H-benzoimidazol-2-yl)-isothiazol-4-yl]-urea; or
1-(2,6-difluorophenyl)-3-[3-(5-morpholin-4-ylmethyl-1H-benzoimidazol-2-yl)-isothiazol-4-yl]-urea;
or a salt, tautomer, N-oxide or solvate thereof.

139. A pharmaceutical composition comprising at least one compound as defined in claim 109, or a salt, solvate, tautomer or N-oxide thereof together with one or more pharmaceutically acceptable carriers, adjuvants, excipients, diluents, fillers, buffers, stabilisers, preservatives or lubricants, and optionally other therapeutic or prophylactic agents.

140. A method for treating (or alleviating or reducing the incidence of) a disease or condition comprising or arising from abnormal cell growth in a mammal, which method comprises administering to the mammal a compound according to claim 109, or a salt, solvate, tautomer or N-oxide thereof, in an amount effective in inhibiting abnormal cell growth.

141. A method according to claim 140 wherein the disease state or condition is a cancer.

142. A method according to claim 141 wherein the cancer is a carcinoma of the bladder, breast, colon, kidney, epidermis, liver, lung, oesophagus, gall bladder, ovary, pancreas, stomach, cervix, thyroid, prostate, or skin; a hematopoietic tumour of lymphoid lineage; a hematopoietic tumour of myeloid lineage; thyroid follicular cancer; a tumour of mesenchymal origin; a tumour of the central or peripheral nervous system; melanoma; seminoma; teratocarcinoma; osteosarcoma; xeroderma pigmentosum; keratoctanthoma; thyroid follicular cancer; or Kaposi's sarcoma.

143. A method according to claim 141 wherein the cancer is a leukaemia selected from relapsed or refractory acute myelogenous leukemia, myelodysplastic syndrome, acute lymphocytic leukemia and chronic myelogenous leukemia.

144. A method according to claim 141 wherein the disease state is a cancer selected from breast cancer, ovarian cancer, colon cancer, prostate cancer, oesophageal cancer, squamous cancer, and non-small cell lung carcinomas.

145. A method of treatment of B-cell lymphoma, diffuse large B cell lymphoma or chronic lymphocytic leukaemia by administering to a patient in need of such treatment a compound as defined in claim 109 or a salt, solvate, tautomer or N-oxide thereof.

146. A process for the preparation of a compound of the formula (I) as defined in claim 109; which process comprises:
(A) the cyclisation of a compound of the formula (XII): 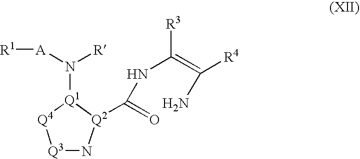
wherein R′ is R⁰ or an N-protecting group, and R⁰, R¹, R³, R⁴ and Q¹ to Q⁴ are as defined in claim 109 provided that the moiety A in R¹-A- contains a group C═O; or
(B) the reaction of a compound of the formula (X) with a compound of the formula (XI): 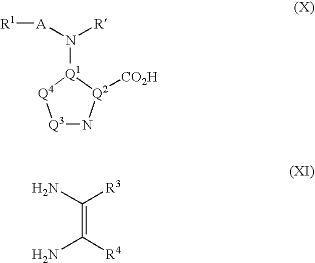
under amide formation and cyclisation conditions; wherein R′ is R⁰ or an N-protecting group, and R⁰, R¹, R³, R⁴ and Q¹ to Q⁴ are as defined in claim 109 provided that the moiety A in R¹-A- contains a group C═O; or
(C) when Q⁴ is S and Q³ is CH; the cyclisation of a compound of the formula (XXII): 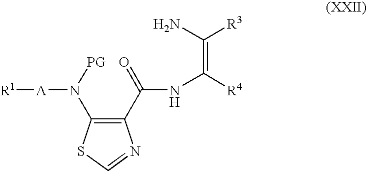
wherein PG is a protecting group and R¹, R³ and R⁴ are as defined in claim 109, and thereafter where required removing the protecting group PG; and optionally wherein the compound of formula (XXII) is formed by the reaction of a compound of the formula (XXI) with a compound of the formula (XI) under amide forming conditions: [chemical structure]
